SEQUENCE LISTING (46 sequences)

SEQ ID NO: 1 - protein, 236 aa, synthetic construct
  REGION 1..236: note = TEV protease mutant
GESLFKGPRDYNPISSSICHLTNESDGHTTSLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMNKPEEPFQPVKEATQLMN

SEQ ID NO: 2 - protein, 242 aa, Tobacco etch virus
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMSKPEEPFQPVKEATQLMNELVYSQ

SEQ ID NO: 3 - protein, 236 aa, synthetic construct
  REGION 1..236: note = TEV protease DM
GESLFKGPRDYNPISSSICHLTNESDGHTTSLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSSDGIFWKHWIQTKDGQCGNPLVSTRDGFIVGIHSASNFTNTNNYFTSVPKNFMELLTNQEAQQWVSGWGLNADSVLWGGHKVFMNKPEEPFQPVKEATQLMN

SEQ ID NO: 4 - protein, 236 aa, synthetic construct
  REGION 1..236: note = TEV mutant clone 1
GESLFKGPRDYNPISSSICHLVNESDGHTASLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSSDGIFWKHWIQTKNGQCGSPLVSTRDGFIVGIHSASNFTNTNNYFTSVPKNFMELLTNQEAQQWVSGWGLNADSVLWGGHKVFFNKPEEPFQPVKEATQLMN

SEQ ID NO: 5 - protein, 236 aa, synthetic construct
  REGION 1..236: note = TEV mutant clone 4
GESLFKGPRDYNPISSSICHLSNESDGHTASLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSSDGIFWKHWIQTKNGQCGSPLVSTRDGFIVGIHSASNFTNTNNYFTSVPKNFMELLTNQEAQQWVSGWGLNADSVLWGGHKVFFNKPEEPFQPVKEATQLMN

SEQ ID NO: 6 - protein, 236 aa, synthetic construct
  REGION 1..236: note = TEV mutant clone 2
GESLFKGPRDYNPISSSICHLINESDGHTASLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWGLNADSVLWGGHKVFFNKPEEPFQPVKEATQLMN

SEQ ID NO: 7 - protein, 236 aa, synthetic construct
  REGION 1..236: note = TEV mutant clone 8
GESLFKGPRDYNPISSSICHLINESDGHTASLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSSDGIFWKHWIQTKRGQCGSPLVSTRDGFIVGIHSASNFTNTNNYFTSVPKNFMELLTNQEAQQWVSGWGLNADSVLWGGHKVFFNKPEEPFQPVKEATQLMN

SEQ ID NO: 8 - protein, 236 aa, synthetic construct
  REGION 1..236: note = TEV mutant clone 11
GESLFKGPRDYNPISSSICHLYNESDGHTSSLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSSDGIFWKHWIQTKNGQCGSPLVSTRDGFIVGIHSASNFTNTNNYFTSVPKNFMELLTNQEAQQWVSGWGLNADSVLWGGHKVFFNKPEEPFQPVKEATQLMN

SEQ ID NO: 9 - protein, 236 aa, synthetic construct
  REGION 1..236: note = TEV mutant clone 19
GESLFKGPRDYNPISSSICHLINESDGHTSSLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSSDGIFWKHWIQTKYGQCGSPLVSTRDGFIVGIHSASNFTNTNNYFTSVPKNFMELLTNQEAQQWVSGWGLNADSVLWGGHKVFFNKPEEPFQPVKEATQLMN

SEQ ID NO: 10 - protein, 236 aa, synthetic construct
  REGION 1..236: note = TEV mutant clone 20
GESLFKGPRDYNPISSSICHLYNESDGHTSSLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSSDGIFWKHWIQTKRGQCGSPLVSTRDGFIVGIHSASNFTNTNNYFTSVPKNFMELLTNQEAQQWVSGWGLNADSVLWGGHKVFFNKPEEPFQPVKEATQLMN

SEQ ID NO: 11 - protein, 236 aa, synthetic construct
  REGION 1..236: note = TEV mutant clone 16
GESLFKGPRDYNPISSSICHLINESDGHTSSLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWGLNADSVLWGGHKVFFNKPEEPFQPVKEATQLMN

SEQ ID NO: 12 - protein, 236 aa, synthetic construct
  REGION 1..236: note = TEV mutant clone 17
GESLFKGPRDYNPISSSICHLINESDGHTSSLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSSDGIFWKHWIQTKRGQCGSPLVSTRDGFIVGIHSASNFTNTNNYFTSVPKNFMELLTNQEAQQWVSGWGLNADSVLWGGHKVFFNKPEEPFQPVKEATQLMN

SEQ ID NO: 13 - protein, 236 aa, synthetic construct
  REGION 1..236: note = TEV mutant clone P1
GESLFKGPRDYNPISSSICHLTNESDGHTTSLYGIGFGPFIITNKHLFRRNNGTLLVQSLHGVFKVKDTTTLQLHLVDGRDMIIIRMPKDFPPFPQKLKFREPQREERICLVTTNFQTKSMSSMVSDTSCTFPSGDGTFWKHWIQTKDGQCGNPLVSTRDGFIVGIHSASNFTNTNNYFTSVPKNFMELLTNQEAQQWVSGWGLNADSVLWGGHKVFMNKPEEPFQPVKEATQLMN

SEQ ID NO: 14 - protein, 236 aa, synthetic construct
  REGION 1..236: note = TEV mutant clone P3
GESLCKGPRDYNPISSSICHLTNESDGHTTSLYGIGFGPFIITNKHLFRRNNGTLLVQSWHGVFKVKDTTTLQLHLVDGRDMIIIRMPKDFPPFPQKLKFREPQREERICLVTTNFQTKSMSSMVSDTSCTFPSGDGHFWKHWIQTKDGQCGNPLVSTRDGFIVGIHSASNFTNTNNYFTSVPKNFMELLTNQEAQQWVSGWGLNADSVCWGGHKVFMNKPEEPFQPVKEATQLMN

SEQ ID NO: 15 - protein, 236 aa, synthetic construct
  REGION 1..236: note = TEV mutant clone P4
GESLCKGPRDYNPISSSICHLTNESDGHTTSLYGIGFGPFIITNKHLFRRNNGTLLVQSWHGVFKVKDTTTLQLHPVDGRDMIIIRMPKDFPPFPQKLKFREPQREERICLVTTNFQTKSMSSMVSDTSTTFPSGDGHFWKHWIQTKDGQCGNPLVSTRDGFIVGIHSASNFTNTNNYFTSVPKNFMELLTNQEAQQWVKGWGLNTDSVCWGGHKVFMNKPEEPFQPVKEATQLMN

SEQ ID NO: 16 - protein, 236 aa, synthetic construct
  REGION 1..236: note = TEV mutant clone P5
GESLCKGPRDYNPISSSICHLTNESDGHTTSLYGIGFGPFIITNKHLFRRNNGTLLVQSWHGVFKVKDTTTLQLHPVEGRDMIIIRMPKDFPPFPQKLKFREPQREERICLVGTNFQEKSMSSMVSETSTTFPSGDGHFWKHWIQTKDGQCGNPLVSTRDGFIVGIHSASNFTNTNNYFTSVPKNFMELLTNQEAQQWVKGWGLNTDSVCWGGHKVFMNKPEEPFQPVKEATQLMN

SEQ ID NO: 17 - protein, 236 aa, synthetic construct
  REGION 1..236: note = TEV mutant clone P1rev
GESLFKGPRDYNPISSSICHLTNESDGHTTSLYGIGFGPFIITNKHLFRRNNGTLLVQSLHGVFKVKDTTTLQLHLVDGRDMIIIRMPKDFPPFPQKLKFREPQREERICLVTTNFQTKSMSSMVSDTSCTFPSGDGTFWKHWIQTKDGQCGSPLVSTRDGFIVGIHSASNFTNTNNYFTSVPKNFMELLTNQEAQQWVSGWGLNADSVLWGGHKVFMNKPEEPFQPVKEATQLMN

SEQ ID NO: 18 - protein, 7 aa, synthetic construct
  REGION 1..7: note = TEV recognition site
  SITE 7: note = misc_feature - Xaa can be any naturally occurring amino acid
ENLYFQX

SEQ ID NO: 19 - protein, 10 aa, synthetic construct
  REGION 1..10: note = Gly peptide substrate
ENLYFQGGGK

SEQ ID NO: 20 - protein, 10 aa, synthetic construct
  REGION 1..10: note = Val peptide substrate
ENLYFQVGGK

SEQ ID NO: 21 - protein, 14 aa, synthetic construct
  REGION 1..14: note = N terminal tag
GTHHHHHHGSGSGT

SEQ ID NO: 22 - protein, 10 aa, synthetic construct
  REGION 1..10: note = TEV substrate peptide
  VARIANT 7: note = Xaa can be any naturally occurring amino acid
ENLYFQXGGK

SEQ ID NO: 23 - protein, 250 aa, synthetic construct
GTHHHHHHGSGSGTGESLHKGPRDYNNIASSICLLTNESDGHTTSLYGIGFGPYIITNKHLFRRNNGTLKVQSHHGVFKVPDTTTLQLHFVEGRDMIIIRMPKDFPPFPQKLKFREPQRNERICLVGTNFQEKSLSTMVSETSTTYPVGDGTFWKHWIQTKDGQCGNPMVSTKDGAIVGIHSASNFTNTNNYFTSIPKNFMELLTNPEAQDWVKGWGYNADSVLWGGHKVFMNKPEEPFQPVKEATQLMN

SEQ ID NO: 24 - protein, 236 aa, synthetic construct
GESLFKGPRDYNPISSSICHLTNESDGHTTSLYGIGFGPFIITNKHLFRRNNGTLLVQSLHGVFKVKDTTTLQLHLVDGRDMIIIRMPKDFPPFPQKLKFREPQREERICLVTTNFQTKSMSSMVSDTSCTFPSSDGIFWKHWIQTKDGQCGNPLVSTRDGFIVGIHSASNFTNTNNYFTSVPKNFMELLTNQEAQQWVSGWGLNADSVLWGGHKVFMNKPEEPFQPVKEATQLMN

SEQ ID NO: 25 - protein, 236 aa, synthetic construct
GESLFKGPRDYNPISSSICHLTNESDGHTTSLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSGDGIFWKHWIQTKDGQCGNPLVSTRDGFIVGIHSASNFTNTNNYFTSVPKNFMELLTNQEAQQWVSGWGLNADSVLWGGHKVFMNKPEEPFQPVKEATQLMN

SEQ ID NO: 26 - protein, 236 aa, synthetic construct
GESLFKGPRDYNPISSSICHLTNESDGHTTSLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSSDGTFWKHWIQTKDGQCGNPLVSTRDGFIVGIHSASNFTNTNNYFTSVPKNFMELLTNQEAQQWVSGWGLNADSVLWGGHKVFMNKPEEPFQPVKEATQLMN

SEQ ID NO: 27 - protein, 236 aa, synthetic construct
GESLFKGPRDYNPISSSICHLTNESDGHTTSLYGIGFGPFIITNKHLFRRNNGTLLVQSLHGVFKVKDTTTLQLHLVDGRDMIIIRMPKDFPPFPQKLKFREPQREERICLVTTNFQTKSMSSMVSDTSCTFPSGDGIFWKHWIQTKDGQCGNPLVSTRDGFIVGIHSASNFTNTNNYFTSVPKNFMELLTNQEAQQWVSGWGLNADSVLWGGHKVFMNKPEEPFQPVKEATQLMN

SEQ ID NO: 28 - protein, 236 aa, synthetic construct
GESLFKGPRDYNPISSSICHLTNESDGHTTSLYGIGFGPFIITNKHLFRRNNGTLLVQSLHGVFKVKDTTTLQLHLVDGRDMIIIRMPKDFPPFPQKLKFREPQREERICLVTTNFQTKSMSSMVSDTSCTFPSSDGTFWKHWIQTKDGQCGNPLVSTRDGFIVGIHSASNFTNTNNYFTSVPKNFMELLTNQEAQQWVSGWGLNADSVLWGGHKVFMNKPEEPFQPVKEATQLMN

SEQ ID NO: 29 - protein, 236 aa, synthetic construct
GESLFKGPRDYNPISSSICHLTNESDGHTTSLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSGDGTFWKHWIQTKDGQCGNPLVSTRDGFIVGIHSASNFTNTNNYFTSVPKNFMELLTNQEAQQWVSGWGLNADSVLWGGHKVFMNKPEEPFQPVKEATQLMN

SEQ ID NO: 30 - protein, 236 aa, synthetic construct
GESLFKGPRDYNPISSSICHLTNESDGHTTSLYGIGFGPFIITNKHLFRRNNGTLLVQSLHGVFKVKDTTTLQLHLVDGRDMIIIRMPKDFPPFPQKLKFREPQREERICLVTTNFQTKSMSSMVSDTSCTFPSGDGTFWKHWIQTKDGQCGNPLVSTRDGFIVGIHSASNFTNTNNYFTSVPKNFMELLTNQEAQQWVSGWGLNADSVLWGGHKVFFNKPEEPFQPVKEATQLMN

SEQ ID NO: 31 - protein, 236 aa, synthetic construct
GESLFKGPRDYNPISSSICHLTNESDGHTTSLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWGLNADSVLWGGHKVFMNKPEEPFQPVKEATQLMN

SEQ ID NO: 32 - protein, 250 aa, synthetic construct
GTHHHHHHGSGSGTGESLFKGPRDYNPISSSICHLTNESDGHTTSLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWGLNADSVLWGGHKVFMNKPEEPFQPVKEATQLMN

SEQ ID NO: 33 - protein, 250 aa, synthetic construct
GTHHHHHHGSGSGTGESLFKGPRDYNPISSSICHLVNESDGHTASLYGIGFGPFIITNKHLFRRNNGTLLVQSLHGVFKVKDTTTLQQHLVDGRDMIIIRMPKDFPPFPQKLKFREPQREERICLVTTNFQTKSMSSMVSDTSCTFPSSDGIFWKHWIQTKNGQCGSPLVSTRDGFIVGIHSASNFTNTNNYFTSVPKNFMELLTNQEAQQWVSGWGLNADSVLWGGHKVFFNKPEEPFQPVKEATQLMN

SEQ ID NO: 34 - protein, 6 aa, synthetic construct
ENLYFQ

SEQ ID NO: 35 - protein, 12 aa, synthetic construct
GGSENLYFQSVS

SEQ ID NO: 36 - protein, 7 aa, synthetic construct
ENLYFQS

SEQ ID NO: 37 - protein, 10 aa, synthetic construct
TENLYFQSGT

SEQ ID NO: 38 - protein, 12 aa, synthetic construct
ACEENLYFQGGK

SEQ ID NO: 39 - protein, 12 aa, synthetic construct
ACEENLYFQVGK

SEQ ID NO: 40 - protein, 17 aa, synthetic construct
GHHHHHHGESLFKGPRD

SEQ ID NO: 41 - protein, 24 aa, synthetic construct
GTHHHHHHGSGSGTGESLFKGPRD

SEQ ID NO: 42 - protein, 18 aa, synthetic construct
NKPEEPFQPVKEATQLMN

SEQ ID NO: 43 - protein, 9 aa, synthetic construct
HTTSLYGIG

SEQ ID NO: 44 - protein, 5 aa, synthetic construct
FMSKP

SEQ ID NO: 45 - protein, 7 aa, synthetic construct
ENLYFQG

SEQ ID NO: 46 - protein, 7 aa, synthetic construct
ENLYFQV

(19) United States
(12) Patent Application Publication
(10) Pub. No.: US 2026/0139239 A1
(43) Pub. Date: May 21, 2026

(54) VARIANTS OF TEV PROTEASE AND USES THEREOF

(71) Applicants: NUMAFERM GmbH, Düsseldorf (DE); Technische Universität München, München (DE)

(72) Inventors: Christian Schwarz, Neuss (DE); Martin Bemelmans, Turnhout (BE); Volker Sieber, Straubing (DE); Bach-Ngan Wetzel, Düsseldorf (DE)

(21) Appl. No.: 19/102,291

(22) PCT Filed: Aug. 9, 2023
(86) PCT No.: PCT/EP2023/072079

§ 371 Date: Feb. 7, 2025

(30) Foreign Application Priority Data
Aug. 9, 2022 (EP) 22189465.2

(51) Int. Cl. C12N 9/50; C07K 14/635; C12P 21/06

(52) U.S. Cl. CPC C12N 9/506; C07K 14/635; C12P 21/06; C07K 2319/50; C12Y 304/22044

(57) ABSTRACT

The present invention relates to variants of TEV protease that have—compared to the wildtype enzyme—increased stability and catalytic activity as well as altered substrate specificity. The invention further relates to compositions comprising these variants as well as uses thereof and methods in which these variants are employed.

### REFERENCE TO SEQUENCE LISTING

The present application is being filed along with a Sequence Listing in electronic format. The Sequence Listing is provided as a file entitled MAIW032.003APC.xml, which was created and last modified on Dec. 26, 2025, and is 56,129 bytes in size. The information in the electronic Sequence Listing is hereby incorporated by reference in its entirety.

### FIELD OF THE INVENTION

The present invention lies in the field of molecular biology, specifically the design and optimization of enzyme variants. Specifically, the present invention is directed to variants of TEV protease that have—compared to the wildtype enzyme—increased stability and catalytic activity as well as altered substrate specificity. The invention further relates to compositions comprising these variants as well as uses thereof and methods in which these variants are employed.

### BACKGROUND OF THE INVENTION

Proteases are an important tool in modern biotechnology and widely used to generate peptides, digest polypeptides, for example for sequencing applications, and cleave undesired amino acid stretches, such as peptide tags used for purification, from the peptide or polypeptide of interest.

To date, one of the most frequently used proteases in such applications is TEV protease, a cysteine protease derived from Tobacco etch virus (TEV) with a molecular weight of 27 kDa. The widespread use of TEV protease is mainly due to its high sequence specificity, since it recognizes a 7 amino acid long consensus motif and cleaves the peptide bond between the penultimate and ultimate amino acid, i.e. between amino acids 6 and 7 of the motif. Further advantages are that it is highly active in mammalian cytosol and requires no cofactors. The high sequence specificity minimizes its activity towards other proteins and allows its use not only in vitro methods, but also in intracellular applications.

Despite its many advantages, the use of TEV protease is however hampered by its slow catalysis. Even for its optimal recognition sequence motif, the catalytic activity is significantly lower than that of other proteases, such as trypsin and subtilisin. While there have been some efforts to improve catalytic activity of TEV protease by directed evolution approaches, such as described in international patent publication WO 2021/062063 A1, there is still need for further improved TEV protease variants that may be used in various biotechnological applications and overcome the drawbacks of the wildtype enzyme.

### SUMMARY OF THE INVENTION

The present invention is based on the inventors' surprising finding of variants of TEV protease that exhibit increased catalytic activity and stability and are thus better suited for a variety of biotechnological applications, such as the cleavage of fusion proteins.

In a first aspect, the present invention therefore relates to a polypeptide comprising an amino acid sequence that has at least 80%, at least 85%, at least 90%, at least 95%, at least 97.5%, at least 99% or at least 99.5% sequence identity over its entire length with the amino acid sequence set forth in SEQ ID NO:1 and comprises one or more amino acid substitution(s) selected from the group consisting of: M218F, T22V/I, T30A, D148N/R, Q74L, and S135G, wherein the positions 17, 68, 77, 219 (scaffold), 46, 81 and 151 (catalytic triad) are invariable, wherein the positional numbering is according to SEQ ID NO:1, or a functional fragment thereof.

In various embodiments, the polypeptide further comprises any one or more of the amino acid substitution(s) I138T, S153N and R203G, wherein the positional numbering is according to SEQ ID NO:1.

In various embodiments, the amino acid sequence that has at least 80%, at least 85%, at least 90%, at least 95%, at least 97.5%, at least 99% or at least 99.5% sequence identity over its entire length with the amino acid sequence set forth in SEQ ID NO:1 at its C-Terminus retains the deletion of amino acids 237-242 relative to the TEV protease wildtype sequence set forth in SEQ ID NO:2.

In various embodiments, the functional fragment is at least 202 amino acids in length and comprises amino acid residues corresponding to those at positions 17 to 218 of SEQ ID NO:1.

In various embodiments, the polypeptide comprises
(1) the amino acid substitution M218F and optionally any one or more of T22V/I, T30A, D148N/R, Q74L, and S135G;
(2) the amino acid substitution M218F and optionally any one or more of T22V/I, T30A, D148N/R, Q74L, S135G, I138T, S153N and R203G;
(3) the amino acid substitution M218F and any two, any three, any four, or all five of T22V/I, T30A, D148N/R, Q74L, and S135G;
(4) the amino acid substitution M218F and any two, any three, any four, any five, any six, any seven, or all 8 of T22V/I, T30A, D148N/R, Q74L, S135G, I138T, S153N and R203G;
(5) the amino acid substitutions M218F and T22V/I and optionally any one, any two, any three, or all four of T30A, D148N/R, Q74L, and S135G;
(6) the amino acid substitutions M218F and T22V/I and optionally any one, any two, any three, any four, any five, any six, or all seven of T30A, D148N/R, Q74L, S135G, I138T, S153N and R203G;
(7) the amino acid substitutions M218F and T30A and optionally any one, any two, any three, or all four of T22V/I, D148N/R, Q74L, and S135G;
(8) the amino acid substitutions M218F and T30A and optionally any one, any two, any three, any four, any five, any six, or all seven of T22V/I, D148N/R, Q74L, S135G, I138T, S153N and R203G;
(9) the amino acid substitutions M218F and D148N/R and optionally any one, any two, any three, or all four of T22V/I, T30A, Q74L, and S135G;
(10) the amino acid substitutions M218F and D148N/R and optionally any one, any two, any three, any four, any five, any six, or all seven of T22V/I, T30A, Q74L, S135G, I138T, S153N and R203G;
(11) the amino acid substitutions M218F and Q74L and optionally any one, any two, any three, or all four of T22V/I, T30A, D148N/R, and S135G;
(12) the amino acid substitutions M218F and Q74L and optionally any one, any two, any three, any four, any five, any six, or all seven of T22V/I, T30A, D148N/R, S135G, I138T, S153N and R203G;
(13) the amino acid substitutions M218F and S135G and optionally any one, any two, any three, or all four of T22V/I, T30A, D148N/R, and Q74L;
(14) the amino acid substitutions M218F and S135G and optionally any one, any two, any three, any four, any five, any six, or all seven of T22V/I, T30A, D148N/R, Q74L, I138T, S153N and R203G;
(15) the amino acid substitutions M218F and I138T and optionally any one, any two, any three, any four, any five, any six, or all seven of T22V/I, T30A, D148N/R, Q74L, S135G, S153N and R203G;
(16) the amino acid substitutions M218F and S153N and optionally any one, any two, any three, any four, any five, any six, or all seven of T22V/I, T30A, D148N/R, Q74L, S135G, I138T and R203G;
(17) the amino acid substitutions M218F and R203G and optionally any one, any two, any three, any four, any five, any six, or all seven of T22V/I, T30A, D148N/R, Q74L, S135G, I138T and S153N;
(18) the amino acid substitutions M218F, S153N and R203G and optionally any one, any two, any three, any four, any five, or all six of T22V/I, T30A, D148N/R, Q74L, S135G and I138T;
(19) the amino acid substitution T22V/I and optionally any one or more of T30A, D148N/R, Q74L, S135G, and M218F;
(20) the amino acid substitution T22V/I and optionally any one or more of T30A, D148N/R, Q74L, S135G, I138T, S153N, R203G and M218F;
(21) the amino acid substitution T22V/I and any two, any three, any four, or all five of T30A, D148N/R, Q74L, S135G, and M218F;
(22) the amino acid substitution T22V/I and any two, any three, any four, any five, any six, any seven, or all eight of T30A, D148N/R, Q74L, S135G, I138T, S153N, R203G, and M218F;
(23) the amino acid substitutions T22V/I and T30A and optionally any one, any two, any three, r all four of M218F, D148N/R, Q74L, and S135G;
(24) the amino acid substitution T22V/I and T30A and optionally any one, any two, any three, any four, any five, any six, or all seven of M218F, D148N/R, Q74L, S135G, I138T, S153N and R203G;
(25) the amino acid substitutions T22V/I and D148N/R and optionally any one, any two, any three, or all four of M218F, T30A, Q74L, and S135G;
(26) the amino acid substitution T22V/I and D148N/R and optionally any one, any two, any three, any four, any five, any six, or all seven of M218F, T30A, Q74L, S135G, I138T, S153N and R203G;
(27) the amino acid substitutions T22V/I and Q74L and optionally any one, any two, any three, or all four of M218F, T30A, D148N/R, and S135G;
(28) the amino acid substitutions T22V/I and Q74L and optionally any one, any two, any three, any four, any five, any six, or all seven of M218F, T30A, D148N/R, S135G, I138T, S153N and R203G;
(29) the amino acid substitutions T22V/I and S135G and optionally any one, any two, any three, or all four of M218F, T30A, D148N/R, and Q74L;
(30) the amino acid substitutions T22V/I and S135G and optionally any one, any two, any three, any four, any five, any six, or all seven of M218F, T30A, D148N/R, Q74L, I138T, S153N and R203G;
(31) the amino acid substitutions T22V/I and I138T and optionally any one, any two, any three, any four, any five, any six, or all seven of M218F, T30A, D148N/R, Q74L, S135G, S153N and R203G;
(32) the amino acid substitutions T22V/I and S153N and optionally any one, any two, any three, any four, any five, any six, or all seven of M218F, T30A, D148N/R, Q74L, S135G, I138T and R203G;
(33) the amino acid substitutions T22V/I and R203G and optionally any one, any two, any three, any four, any five, any six, or all seven of M218F, T30A, D148N/R, Q74L, S135G, I138T and S153N;
(34) the amino acid substitutions T22V/I, S153N and R203G and optionally any one, any two, any three, any four, any five, or all six of M218F, T30A, D148N/R, Q74L, S135G and I138T;
(35) the amino acid substitution T30A and optionally any one or more of T22V/I, D148N/R, Q74L, S135G, and M218F;
(36) the amino acid substitution T30A and optionally any one or more of T22V/I, D148N/R, Q74L, S135G, I138T, S153N, R203G and M218F;
(37) the amino acid substitution T30A and any two, any three, any four, or all five of T22V/I, D148N/R, Q74L, S135G, and M218F;
(38) the amino acid substitution T30A and any two, any three, any four, any five, any six, any seven, or all eight of T22V/I, D148N/R, Q74L, S135G, I138T, S153N, R203G, and M218F;
(39) the amino acid substitutions T30A and D148N/R and optionally any one, any two, any three, or all four of M218F, T22V/I, Q74L, and S135G;
(40) the amino acid substitution T30A and D148N/R and optionally any one, any two, any three, any four, any five, any six, or all seven of M218F, T22V/I, Q74L, S135G, I138T, S153N and R203G;
(41) the amino acid substitutions T30A and Q74L and optionally any one, any two, any three, or all four of M218F, T22V/I, D148N/R, and S135G;
(42) the amino acid substitutions T30A and Q74L and optionally any one, any two, any three, any four, any five, any six, or all seven of M218F, T22V/I, D148N/R, S135G, I138T, S153N and R203G;
(43) the amino acid substitutions T30A and S135G and optionally any one, any two, any three, or all four of M218F, T22V/I, D148N/R, and Q74L;
(44) the amino acid substitutions T30A and S135G and optionally any one, any two, any three, any four, any five, any six, or all seven of M218F, T22V/I, D148N/R, Q74L, I138T, S153N and R203G;
(45) the amino acid substitutions T30A and I138T and optionally any one, any two, any three, any four, any five, any six, or all seven of M218F, T22V/I, D148N/R, Q74L, S135G, S153N and R203G;
(46) the amino acid substitutions T30A and S153N and optionally any one, any two, any three, any four, any five, any six, or all seven of M218F, T22V/I, D148N/R, Q74L, S135G, I138T and R203G;
(47) the amino acid substitutions T30A and R203G and optionally any one, any two, any three, any four, any five, any six, or all seven of M218F, T22V/I, D148N/R, Q74L, S135G, I138T and S153N;
(48) the amino acid substitutions T30A, S153N and R203G and optionally any one, any two, any three, any four, any five, or all six of M218F, T22V/I, D148N/R, Q74L, S135G and I138T;
(49) the amino acid substitution D148N/R and optionally any one or more of T22V/I, T30A, Q74L, S135G, and M218F;
(50) the amino acid substitution D148N/R and optionally any one or more of T22V/I, T30A, Q74L, S135G, I138T, S153N, R203G and M218F;
(51) the amino acid substitution D148N/R and any two, any three, any four, or all five of T22V/I, T30A, Q74L, S135G, and M218F;
(52) the amino acid substitution D148N/R and any two, any three, any four, any five, any six, any seven, or all eight of T22V/I, T30A, Q74L, S135G, I138T, S153N, R203G, and M218F;
(53) the amino acid substitutions D148N/R and Q74L and optionally any one, any two, any three, or all four of M218F, T22V/I, T30A, and S135G;
(54) the amino acid substitutions D148N/R and Q74L and optionally any one, any two, any three, any four, any five, any six, or all seven of M218F, T22V/I, T30A, S135G, I138T, S153N and R203G;
(55) the amino acid substitutions D148N/R and S135G and optionally any one, any two, any three, or all four of M218F, T22V/I, T30A, and Q74L;
(56) the amino acid substitutions D148N/R and S135G and optionally any one, any two, any three, any four, any five, any six, or all seven of M218F, T22V/I, T30A, Q74L, I138T, S153N and R203G;
(57) the amino acid substitutions D148N/R and I138T and optionally any one, any two, any three, any four, any five, any six, or all seven of M218F, T22V/I, T30A, Q74L, S135G, S153N and R203G;
(58) the amino acid substitutions D148N/R and S153N and optionally any one, any two, any three, any four, any five, any six, or all seven of M218F, T22V/I, T30A, Q74L, S135G, I138T and R203G;
(59) the amino acid substitutions D148N/R and R203G and optionally any one, any two, any three, any four, any five, any six, or all seven of M218F, T22V/I, T30A, Q74L, S135G, I138T and S153N;
(60) the amino acid substitutions D148N/R, S153N and R203G and optionally any one, any two, any three, any four, any five, or all six of M218F, T22V/I, T30A, Q74L, S135G and I138T;
(61) the amino acid substitution Q74L and optionally any one or more of T22V/I, T30A, D148N/R, S135G, and M218F;
(62) the amino acid substitution Q74L and optionally any one or more of T22V/I, T30A, D148N/R, S135G, I138T, S153N, R203G and M218F;
(63) the amino acid substitution Q74L and any two, any three, any four, or all five of T22V/I, T30A, D148N/R, S135G, and M218F;
(64) the amino acid substitution Q74L and any two, any three, any four, any five, any six, any seven, or all eight of T22V/I, T30A, D148N/R, S135G, I138T, S153N, R203G, and M218F;
(65) the amino acid substitutions Q74L and S135G and optionally any one, any two, any three, or all four of M218F, T22V/I, T30A, and D148N/R;
(66) the amino acid substitutions Q74L and S135G and optionally any one, any two, any three, any four, any five, any six, or all seven of M218F, T22V/I, T30A, D148N/R, I138T, S153N and R203G;
(67) the amino acid substitutions Q74L and I138T and optionally any one, any two, any three, any four, any five, any six, or all seven of M218F, T22V/I, T30A, D148N/R, S135G, S153N and R203G;
(68) the amino acid substitutions Q74L and S153N and optionally any one, any two, any three, any four, any five, any six, or all seven of M218F, T22V/I, T30A, D148N/R, S135G, I138T and R203G;
(69) the amino acid substitutions Q74L and R203G and optionally any one, any two, any three, any four, any five, any six, or all seven of M218F, T22V/I, T30A, D148N/R, S135G, I138T and S153N;
(70) the amino acid substitutions Q74L, S153N and R203G and optionally any one, any two, any three, any four, any five, or all six of M218F, T22V/I, T30A, D148N/R, S135G and I138T;
(71) the amino acid substitution S135G and optionally any one or more of T22V/I, T30A, D148N/R, Q74L, and M218F;
(72) the amino acid substitution S135G and optionally any one or more of T22V/I, T30A, D148N/R, Q74L, I138T, S153N, R203G and M218F;
(73) the amino acid substitution S135G and any two, any three, any four, or all five of T22V/I, T30A, D148N/R, Q74L, and M218F;
(74) the amino acid substitution S135G and any two, any three, any four, any five, any six, any seven, or all eight of T22V/I, T30A, D148N/R, Q74L, I138T, S153N, R203G, and M218F;
(75) the amino acid substitutions S135G and I138T and optionally any one, any two, any three, any four, any five, any six, or all seven of M218F, T22V/I, T30A, D148N/R, Q74L, S153N and R203G;
(76) the amino acid substitutions S135G and S153N and optionally any one, any two, any three, any four, any five, any six, or all seven of M218F, T22V/I, T30A, D148N/R, Q74L, I138T and R203G;
(77) the amino acid substitutions S135G and R203G and optionally any one, any two, any three, any four, any five, any six, or all seven of M218F, T22V/I, T30A, D148N/R, Q74L, I138T and S153N;
(78) the amino acid substitutions S135G, S153N and R203G and optionally any one, any two, any three, any four, any five, or all six of M218F, T22V/I, T30A, D148N/R, Q74L and I138T;
(79) the amino acid substitutions M218F, T22V/I and T30A and any one, any two, or all three of D148N/R, Q74L, and S135G;
(80) the amino acid substitutions M218F, T22V/I and D148N/R and any one, any two, or all three of T30A, Q74L, and S135G;
(81) the amino acid substitutions M218F, T30A and D148N/R and any one, any two, or all three of T22V/I, Q74L, and S135G;
(82) the amino acid substitutions T22V/I, T30A and D148N/R and any one, any two, or all three of M218F, Q74L, and S135G;
(83) the amino acid substitutions M218F, T22V/I, T30A and D148N/R and any one, or both of Q74L and S135G; or
(84) the amino acid substitutions M218F, T22V/I, T30A and D148N/R and any one, any two, any three, any four, or all five of Q74L, S135G, I138T, S153N, and R203G.

In various embodiments of the polypeptides disclosed herein, T22V/I is T22V or T22V/I is T22I. In various embodiments of the polypeptides disclosed herein D148N/R is D148R or D148N/R is D148N.

The polypeptides of the invention may be isolated polypeptides or purified polypeptides.

In various embodiments, the polypeptide is up to 236 amino acids in length, for example 202 or 219 amino acids in length.

In various embodiments, the polypeptide has protease activity, in particular TEV protease activity.

In various embodiments, the polypeptide comprises or consists of the amino acid sequence set forth in any one of SEQ ID Nos. 4 to 17.

In another aspect, the polypeptide according to the invention comprises an amino acid sequence that has at least 80%, at least 85%, at least 90%, at least 95%, at least 97.5%, at least 99% or at least 99.5% sequence identity over its entire length with the amino acid sequence set forth in SEQ ID NO:1 and comprises the amino acid substitution Q74L/V/I/F/W/Y/C and optionally any one or more of S135G/F, I138T, S153N/C/I/V, R203G/Q and M218F/I/W/L; wherein the amino acids at positions 68, 77, (scaffold), 46, 81 and 151 (catalytic triad) are invariable, and the amino acid at position 17 is S or A and the amino acid at position 219 is N/V/D/E/P/K (scaffold), wherein the positional numbering is according to SEQ ID NO:1, or a functional fragment thereof, wherein the polypeptide or the functional fragment has protease activity.

In various embodiments, the amino acid sequence that has at least 80%, at least 85%, at least 90%, at least 95%, at least 97.5%, at least 99% or at least 99.5% sequence identity over its entire length with the amino acid sequence set forth in SEQ ID NO:1 retains the deletion of amino acids 237-242 at its C-terminus relative to the TEV protease wildtype sequence set forth in SEQ ID NO:2.

In various embodiments, the functional fragment is at least 202 amino acids in length and comprises amino acid residues corresponding to those at positions 17 to 218 of SEQ ID NO:1.

The polypeptide may comprise, in various embodiments,
(1) the amino acid substitutions Q74L/V/I/F/W/Y/C and S135G/F, and optionally any one or more of I138T, S153N/C/I/V, R203G/Q and M218F/I/W/L;
(2) the amino acid substitutions Q74L and S135G, and optionally any one or more of I138T, S153N, R203G and M218F;
(3) the amino acid substitutions Q74L/V/I/F/W/Y/C and I138T, and optionally any one or more of S135G/F, S153N/C/I/V, R203G/Q and M218F/I/W/L;
(4) the amino acid substitutions Q74L and I138T, and optionally any one or more of S135G, S153N, R203G and M218F;
(5) the amino acid substitutions Q74LN/I/F/W/Y/C and S153N/C/IN, and optionally any one or more of S135G/F, I138T, R203G/Q and M218F/I/W/L;
(6) the amino acid substitutions Q74L, and S153N, and optionally any one or more of S135G, I138T, R203G and M218F;
(7) the amino acid substitutions Q74L/V/I/F/W/Y/C and R203G/Q, and optionally any one or more of S135G/F, I138T, S153N/C/I/V and M218F/I/W/L;
(8) the amino acid substitutions Q74L, and R203G, and optionally any one or more of S135G, I138T, S153N and M218F;
(9) the amino acid substitutions Q74L/V/I/F/W/Y/C and M218F/I/W/L, and optionally any one or more of S135G/F, I138T, S153N/C/I/V and R203G/Q;
(10) the amino acid substitutions Q74L, and M218F, and optionally any one or more of S135G, I138T, S153N and R203G;
(11) the amino acid substitutions Q74L/V/I/F/W/Y/C, S135G/F and I138T, and optionally any one, any two or three of S153N/C/I/V, R203G/Q and M218F/I/W/L;
(12) the amino acid substitutions Q74L, S135G and I138T, and optionally any one, any two or three of S153N, R203G and M218F;
(13) the amino acid substitutions Q74L/V/I/F/W/Y/C, S135G/F and S153N/C/I/V, and optionally any one, any two or three of I138T, R203G/Q and M218F/I/W/L;
(14) the amino acid substitutions Q74L, S135G, and S153N, and optionally any one, any two or three of I138T, R203G and M218F;
(15) the amino acid substitutions Q74L/V/I/F/W/Y/C, S135G/F and R203G/Q, and optionally any one, any two or three of I138T, S153N/C/I/V and M218F/I/W/L;
(16) the amino acid substitutions Q74L, S135G, and R203G, and optionally any one, any two or three of I138T, S153N and M218F;
(17) the amino acid substitutions Q74L/V/I/F/W/Y/C, S135G/F and M218F/I/W/L, and optionally any one, any two or three of I138T, S153N/C/I/V and R203G/Q;
(18) the amino acid substitutions Q74L, S135G, and M218F, and optionally any one, any two or three of I138T, S153N and R203G;
(19) the amino acid substitutions Q74L/V/I/F/W/Y/C, I138T and S153N/C/I/V, and optionally any one, any two or three of S135G/F, R203G/Q and M218F/I/W/L;
(20) the amino acid substitutions Q74L, I138T and S153N, and optionally any one, any two or three of S135G, R203G and M218F;
(21) the amino acid substitutions Q74L/V/I/F/W/Y/C, I138T and R203G/Q, and optionally any one, any two or three of S135G/F, S153N/C/I/V and M218F/I/W/L;
(22) the amino acid substitutions Q74L, I138T and R203G, and optionally any one, any two or three of S135G, S153N and M218F;
(23) the amino acid substitutions Q74L/V/I/F/W/Y/C, I138T and M218F/I/W/L, and optionally any one, any two or three of S135G/F, S153N/C/I/V and R203G/Q;
(24) the amino acid substitutions Q74L, I138T and M218F, and optionally any one, any two or three of S135G, S153N and R203G;
(25) the amino acid substitutions Q74L/V/I/F/W/Y/C, S153N/C/I/V and R203G/Q, and optionally any one, any two or three of S135G/F, I138T, M218F/I/W/L;
(26) the amino acid substitutions Q74L, S153N, and R203G, and optionally any one, any two or three of S135G, I138T and M218F;
(27) the amino acid substitutions Q74L/V/I/F/W/Y/C, S153N/C/I/V and M218F/I/W/L, optionally any one, any two or three of S135G/F, I138T, R203G/Q;
(28) the amino acid substitutions Q74L, S153N, and M218F, and optionally any one, any two or three of S135G, I138T and R203G;
(29) the amino acid substitutions Q74L/V/I/F/W/Y/C, R203G/Q and M218F/I/W/L, and optionally any one, any two or three of S135G/F, I138T, S153N/C/I/V;
(30) the amino acid substitutions Q74L, R203G, and M218F, and optionally any one, any two or three of S135G, I138T and S153N;
(31) the amino acid substitutions Q74L/V/I/F/W/Y/C, S135G/F, I138T and S153N/C/IN, and optionally any one or two of R203G/Q and M218F/I/W/L;
(32) the amino acid substitutions Q74L, S135G, I138T and S153N, and optionally any one or two of R203G and M218F;
(33) the amino acid substitutions Q74L/V/I/F/W/Y/C, S135G/F, I138T and R203G/Q, and optionally any one or two of S153N/C and M218F/I/W/L;
(34) the amino acid substitutions Q74L, S135G, I138T and R203G, and optionally any one or two of S153N and M218F;
(35) the amino acid substitutions Q74L/V/I/F/W/Y/C, S135G/F, I138T and M218F/I/W/L, and optionally any one or two of S153N/C/IN and R203G/Q;
(36) the amino acid substitutions Q74L, S135G, I138T and M218F, and optionally any one or two of S153N and R203G;
(37) the amino acid substitutions Q74L/V/I/F/W/Y/C, S135G/F, S153N/C/I/V, and R203G/Q, and optionally any one or two of I138T and M218F/I/W/L;
(38) the amino acid substitutions Q74L, S135G, S153N and R203G, and optionally any one or two of I138T and M218F;
(39) the amino acid substitutions Q74L/V/I/F/W/Y/C, S135G/F, S153N/C/I/V and M218F/I/W/L, and optionally any one or two of I138T and R203G/Q;
(40) the amino acid substitutions Q74L, S135G, S153N and M218F, and optionally any one or two of I138T and R203G;
(41) the amino acid substitutions Q74L/V/I/F/W/Y/C, I138T, S153N/C/I/V and R203G/Q, and optionally any one or two of S135G/F and M218F/I/W/L;
(42) the amino acid substitutions Q74L, I138T, S153N, and R203G, and optionally any one or two of S135G and M218F;
(43) the amino acid substitutions Q74L/V/I/F/W/Y/C, I138T, S153N/C/I/V and M218F/I/W/L, and optionally any one or two of S135G/F and R203G/Q;
(44) the amino acid substitutions Q74L, I138T, S153N, and M218F, and optionally any one or two of S135G and R203G;
(45) the amino acid substitutions Q74L/V/I/F/W/Y/C, S153N/C, R203G/Q and M218F/I/W/L, and optionally any one or two of S135G/F and I138T;
(46) the amino acid substitutions Q74L, S153N, R203G, and M218F, and optionally any one or two of S135G and I138T;
(47) the amino acid substitutions Q74L/V/I/F/W/Y/C, S135G/F, R203G/Q, and M218F/I/W/L, and optionally any one or two of I138T and S153N/C/I/V;
(48) the amino acid substitutions Q74L, S135G, R203G, and M218F, and optionally any one or two of I138T and S153N;
(49) the amino acid substitutions Q74L/V/I/F/W/Y/C, S135G/F, I138T, S153N/C/I/V and R203G/Q, and optionally M218F/I/W/L;
(50) the amino acid substitutions Q74L, S135G, I138T, S153N, and R203G, and optionally M218F;
(51) the amino acid substitutions Q74L/V/I/F/W/Y/C, S135G/F, I138T, S153N/C/I/V and M218F/I/W/L, and optionally R203G/Q;
(52) the amino acid substitutions Q74L, S135G, I138T, S153N, and M218F, and optionally R203G;
(53) the amino acid substitutions Q74L/V/I/F/W/Y/C, S135G/F, I138T, R203G/Q and M218F/I/W/L, and optionally S153N/C/I/V;
(54) the amino acid substitutions Q74L, S135G, I138T, R203G, and M218F, and optionally S153N;
(55) the amino acid substitutions Q74L/V/I/F/W/Y/C, S135G/F, S153N/C/I/V, R203G/Q and M218F/I/W/L, and optionally I138T;
(56) the amino acid substitutions Q74L, S135G, S153N, R203G, and M218F, and optionally I138T;
(57) the amino acid substitutions Q74L/V/I/F/W/Y/C, I138T, S153N/C/I/V, R203G/Q and M218F/I/W/L, and optionally S135G/F;
(58) the amino acid substitutions Q74L, I138T, S153N, R203G and M218F, and optionally S135G;
(59) the amino acid substitutions Q74L/V/I/F/W/Y/C, S135G/F, I138T, S153N/C/I/V, R203G/Q and M218F/I/W/L; or
(60) the amino acid substitutions Q74L, S135G, I138T, S153N, R203G and M218F.

Preferably, the polypeptide comprises in various embodiments
(1) the amino acid substitutions Q74L/V/I/F/W/Y/C, S153N/C/I/V and R203G/Q;
(2) the amino acid substitutions Q74L, S153N and R203G;
(3) the amino acid substitutions Q74L/V/I/F/W/Y/C, S135G/F, S153N/C/I/V and R203G/Q;
(4) the amino acid substitutions Q74L, S135G, S153N and R203G;
(5) the amino acid substitutions Q74L/V/I/F/W/Y/C, I138T, S153N/C/I/V and R203G/Q;
(6) the amino acid substitutions Q74L, I138T, S153N and R203G;
(7) the amino acid substitutions Q74L/V/I/F/W/Y/C, S135G/F, I138T, S153N/C/I/V and R203G/Q;
(8) the amino acid substitutions Q74L, S135G, I138T, S153N and R203G;
(9) the amino acid substitutions Q74L/V/I/F/W/Y/C, S135G/F, I138T and R203G/Q;
(10) the amino acid substitutions Q74L, S135G, I138T and R203G;
(11) the amino acid substitutions Q74L/V/I/F/W/Y/C, S135G/F, I138T, S153N/C/I/V, R203G/Q and M218F/I/W/L; or
(12) the amino acid substitutions Q74L, S135G, I138T, S153N, R203G and M218F.

In another aspect, the polypeptide according to the invention comprises an amino acid sequence that has at least 80%, at least 85%, at least 90%, at least 95%, at least 97.5%, at least 99% or at least 99.5% sequence identity over its entire length with the amino acid sequence set forth in SEQ ID NO:1 and comprises the amino acid substitution S135G/F and optionally any one or more of Q74L/V/I/F/W/Y/C, I138T, S153N/C/I/V, R203G/Q and M218F/I/W/L; wherein the amino acids at positions 68, 77, (scaffold), 46, 81 and 151 (catalytic triad) are invariable, and the amino acid at position 17 is S or A and the amino acid at position 219 is N/V/D/E/P/K (scaffold), wherein the positional numbering is according to SEQ ID NO:1, or a functional fragment thereof, wherein the polypeptide or the functional fragment has protease activity. In such an embodiment, the substitutions, if present, are preferably S135G, Q74L, S153N, R203G and/or M218F.

The polypeptide may comprise, in various embodiments,
(1) the amino acid substitutions S135G/F and I138T, and optionally any one or more of Q74L/V/I/F/W/Y/C, S153N/C/I/V, R203G/Q and M218F/I/W/L;
(2) the amino acid substitutions I138T and S135G, and optionally any one or more of Q74L, S153N, R203G and M218F;
(3) the amino acid substitutions S135G/F and S153N/C/I/V, and optionally any one or more of Q74L/V/I/F/W/Y/C, I138T, R203G/Q and M218F/I/W/L;
(4) the amino acid substitutions S135G and S153N, and optionally any one or more of Q74L, I138T, R203G and M218F;
(5) the amino acid substitutions S135G/F and R203G/Q, and optionally any one or more of Q74L/V/I/F/W/Y/C, I138T, S153N/C/I/V and M218F/I/W/L;
(6) the amino acid substitutions S135G and R203G, and optionally any one or more of Q74L, I138T, S153N and M218F;
(7) the amino acid substitutions S135G/F and M218F/I/W/L, and optionally any one or more of Q74L/V/I/F/W/Y/C, I138T, S153N/C/I/V and R203G/Q;
(8) the amino acid substitutions S135G and M218F, and optionally any one or more of Q74L, I138T, S153N and R203G;
(9) the amino acid substitutions Q74L/V/I/F/W/Y/C, S135G/F and I138T, and optionally any one, any two or three of S153N/C/I/V, R203G/Q and M218F/I/W/L;
(10) the amino acid substitutions Q74L, S135G and I138T, and optionally any one, any two or three of S153N, R203G and M218F;
(11) the amino acid substitutions I138T, S135G/F and S153N/C/I/V, and optionally any one, any two or three of Q74L/V/I/F/W/Y/C, R203G/Q and M218F/I/W/L;
(12) the amino acid substitutions I138T, S135G, and S153N, and optionally any one, any two or three of Q74L, R203G and M218F;
(13) the amino acid substitutions I138T, S135G/F and R203G/Q, and optionally any one, any two or three of Q74L/V/I/F/W/Y/C, S153N/C/I/V and M218F/I/W/L;
(14) the amino acid substitutions I138T, S135G, and R203G, and optionally any one, any two or three of Q74L, S153N and M218F;
(15) the amino acid substitutions I138T, S135G/F and M218F/I/W/L, and optionally any one, any two or three of Q74L/V/I/F/W/Y/C, S153N/C/I/V and R203G/Q;
(18) the amino acid substitutions I138T, S135G, and M218F, and optionally any one, any two or three of Q74L, S153N and R203G;
(19) the amino acid substitutions S135G/F, I138T, S153N/C/I/V, and R203G/Q, and optionally any one or two of Q74L/V/I/F/W/Y/C and M218F/I/W/L;
(20) the amino acid substitutions S135G, I138T, S153N and R203G, and optionally any one or two of Q74L and M218F;
(21) the amino acid substitutions S135G/F, I138T, S153N/C/I/V and M218F/I/W/L, and optionally any one or two of Q74L/V/I/F/W/Y/C and R203G/Q;
(22) the amino acid substitutions S135G, I138T, S153N and M218F, and optionally any one or two of Q74L and R203G;
(23) the amino acid substitutions S135G/F, I138T, S153N/C/I/V and R203G/Q, and optionally any one or two of Q74L/V/I/F/W/Y/C and M218F/I/W/L;
(24) the amino acid substitutions S135G, I138T, S153N, and R203G, and optionally any one or two of Q74L and M218F;
(25) the amino acid substitutions S135G/F, I138T, S153N/C/I/V and M218F/I/W/L, and optionally any one or two of Q74L/V/I/F/W/Y/C and R203G/Q;
(26) the amino acid substitutions S135G, I138T, S153N, and M218F, and optionally any one or two of Q74L and R203G;
(27) the amino acid substitutions S135G/F, S153N/C, R203G/Q and M218F/I/W/L, and optionally any one or two of Q74L/V/I/F/W/Y/C and I138T;
(28) the amino acid substitutions S135G, S153N, R203G, and M218F, and optionally any one or two of Q74L and I138T;
(29) the amino acid substitutions I138T, S135G/F, R203G/Q, and M218F/I/W/L, and optionally any one or two of Q74L/V/I/F/W/Y/C and S153N/C/I/V;
(30) the amino acid substitutions I138T, S135G, R203G, and M218F, and optionally any one or two of Q74L and S153N;
(31) the amino acid substitutions Q74L/V/I/F/W/Y/C, S135G/F, I138T, S153N/C/I/V and R203G/Q, and optionally M218F/I/W/L;
(32) the amino acid substitutions Q74L, S135G, I138T, S153N, and R203G, and optionally M218F;
(33) the amino acid substitutions Q74L/V/I/F/W/Y/C, S135G/F, I138T, S153N/C/I/V and M218F/I/W/L, and optionally R203G/Q;
(34) the amino acid substitutions Q74L, S135G, I138T, S153N, and M218F, and optionally R203G;
(35) the amino acid substitutions Q74L/V/I/F/W/Y/C, S135G/F, I138T, R203G/Q and M218F/I/W/L, and optionally S153N/C/I/V;
(36) the amino acid substitutions Q74L, S135G, I138T, R203G, and M218F, and optionally S153N;
(37) the amino acid substitutions Q74L/V/I/F/W/Y/C, S135G/F, S153N/C/I/V, R203G/Q and M218F/I/W/L, and optionally I138T;
(38) the amino acid substitutions Q74L, S135G, S153N, R203G, and M218F, and optionally I138T;
(39) the amino acid substitutions Q74L/V/I/F/W/Y/C, S135G/F, I138T, S153N/C/I/V, R203G/Q and M218F/I/W/L; or
(40) the amino acid substitutions Q74L, S135G, I138T, S153N, R203G and M218F.

Preferably, the polypeptide comprises in various embodiments
(1) the amino acid substitutions S135G/F, S153N/C/I/V and R203G/Q;
(2) the amino acid substitutions S135G, S153N and R203G;
(3) the amino acid substitutions Q74L/V/I/F/W/Y/C, S135G/F, S153N/C/I/V and R203G/Q;
(4) the amino acid substitutions Q74L, S135G, S153N and R203G;
(5) the amino acid substitutions S135G/F, I138T, S153N/C/I/V and R203G/Q;
(6) the amino acid substitutions S135G, I138T, S153N and R203G;
(7) the amino acid substitutions Q74L/V/I/F/W/Y/C, S135G/F, I138T, S153N/C/I/V and R203G/Q;
(8) the amino acid substitutions Q74L, S135G, I138T, S153N and R203G;
(9) the amino acid substitutions Q74L/V/I/F/W/Y/C, S135G/F, I138T and R203G/Q;
(10) the amino acid substitutions Q74L, S135G, I138T and R203G;
(11) the amino acid substitutions Q74L/V/I/F/W/Y/C, S135G/F, I138T, S153N/C/I/V, R203G/Q and M218F/I/W/L; or
(12) the amino acid substitutions Q74L, S135G, I138T, S153N, R203G and M218F.

In another aspect, the polypeptide according to the invention comprises an amino acid sequence that has at least 80%, at least 85%, at least 90%, at least 95%, at least 97.5%, at least 99% or at least 99.5% sequence identity over its entire length with the amino acid sequence set forth in SEQ ID NO:1 and comprises the amino acid substitution I138T and optionally any one or more of Q74L/V/I/F/W/Y/C, S135G/F, S153N/C/I/V, R203G/Q and M218F/I/W/L; wherein the amino acids at positions 68, 77, (scaffold), 46, 81 and 151 (catalytic triad) are invariable, and the amino acid at position 17 is S or A and the amino acid at position 219 is N/V/D/E/P/K (scaffold), wherein the positional numbering is according to SEQ ID NO:1, or a functional fragment thereof, wherein the polypeptide or the functional fragment has protease activity. In such an embodiment, the substitutions, if present, are preferably S135G, Q74L, S153N, R203G and/or M218F.

The polypeptide may comprise, in various embodiments,
(1) the amino acid substitutions S135G/F and I138T, and optionally any one or more of Q74L/V/I/F/W/Y/C, S153N/C/I/V, R203G/Q and M218F/I/W/L;
(2) the amino acid substitutions I138T and S135G, and optionally any one or more of Q74L, S153N, R203G and M218F;
(3) the amino acid substitutions I138T and S153N/C/I/V, and optionally any one or more of Q74L/V/I/F/W/Y/C, S135G/F, R203G/Q and M218F/I/W/L;
(4) the amino acid substitutions I1b38T and S153N, and optionally any one or more of Q74L, S135G, R203G and M218F;
(5) the amino acid substitutions I138T and R203G/Q, and optionally any one or more of Q74L/V/I/F/W/Y/C, S135G/F, S153N/C/I/V and M218F/I/W/L;
(6) the amino acid substitutions I138T and R203G, and optionally any one or more of Q74L, S135G, S153N and M218F;
(7) the amino acid substitutions I138T and M218F/I/W/L, and optionally any one or more of Q74L/V/I/F/W/Y/C, S135G/F, S153N/C/I/V and R203G/Q;
(8) the amino acid substitutions I138T and M218F, and optionally any one or more of Q74L, S135G, S153N and R203G;
(9) the amino acid substitutions Q74L/V/I/F/W/Y/C, S135G/F and I138T, and optionally any one, any two or three of S153N/C/I/V, R203G/Q and M218F/I/W/L;
(10) the amino acid substitutions Q74L, S135G and I138T, and optionally any one, any two or three of S153N, R203G and M218F;
(11) the amino acid substitutions I138T, S135G/F and S153N/C/I/V, and optionally any one, any two or three of Q74L/V/I/F/W/Y/C, R203G/Q and M218F/I/W/L;
(12) the amino acid substitutions I138T, S135G, and S153N, and optionally any one, any two or three of Q74L, R203G and M218F;
(13) the amino acid substitutions I138T, S135G/F and R203G/Q, and optionally any one, any two or three of Q74L/V/I/F/W/Y/C, S153N/C/I/V and M218F/I/W/L;
(14) the amino acid substitutions I138T, S135G, and R203G, and optionally any one, any two or three of Q74L, S153N and M218F;
(15) the amino acid substitutions I138T, S135G/F and M218F/I/W/L, and optionally any one, any two or three of Q74L/V/I/F/W/Y/C, S153N/C/I/V and R203G/Q;
(18) the amino acid substitutions I138T, S135G, and M218F, and optionally any one, any two or three of Q74L, S153N and R203G;
(19) the amino acid substitutions S135G/F, I138T, S153N/C/I/V, and R203G/Q, and optionally any one or two of Q74L/V/I/F/W/Y/C and M218F/I/W/L;
(20) the amino acid substitutions S135G, I138T, S153N and R203G, and optionally any one or two of Q74L and M218F;
(21) the amino acid substitutions S135G/F, I138T, S153N/C/I/V and M218F/I/W/L, and optionally any one or two of Q74L/V/I/F/W/Y/C and R203G/Q;
(22) the amino acid substitutions S135G, I138T, S153N and M218F, and optionally any one or two of Q74L and R203G;
(23) the amino acid substitutions S135G/F, I138T, S153N/C/I/V and R203G/Q, and optionally any one or two of Q74L/V/I/F/W/Y/C and M218F/I/W/L;
(24) the amino acid substitutions S135G, I138T, S153N, and R203G, and optionally any one or two of Q74L and M218F;
(25) the amino acid substitutions S135G/F, I138T, S153N/C/I/V and M218F/I/W/L, and optionally any one or two of Q74L/V/I/F/W/Y/C and R203G/Q;
(26) the amino acid substitutions S135G, I138T, S153N, and M218F, and optionally any one or two of Q74L and R203G;
(27) the amino acid substitutions I138T, S153N/C, R203G/Q and M218F/I/W/L, and optionally any one or two of Q74L/V/I/F/W/Y/C and S135G/F;
(28) the amino acid substitutions I138T, S153N, R203G, and M218F, and optionally any one or two of Q74L and S135G;
(29) the amino acid substitutions I138T, S135G/F, R203G/Q, and M218F/I/W/L, and optionally any one or two of Q74L/V/I/F/W/Y/C and S153N/C/I/V;
(30) the amino acid substitutions I138T, S135G, R203G, and M218F, and optionally any one or two of Q74L and S153N;
(31) the amino acid substitutions Q74L/V/I/F/W/Y/C, S135G/F, I138T, S153N/C/I/V and R203G/Q, and optionally M218F/I/W/L;
(32) the amino acid substitutions Q74L, S135G, I138T, S153N, and R203G, and optionally M218F;
(33) the amino acid substitutions Q74L/V/I/F/W/Y/C, S135G/F, I138T, S153N/C/I/V and M218F/I/W/L, and optionally R203G/Q;
(34) the amino acid substitutions Q74L, S135G, I138T, S153N, and M218F, and optionally R203G;
(35) the amino acid substitutions Q74L/V/I/F/W/Y/C, S135G/F, I138T, R203G/Q and M218F/I/W/L, and optionally S153N/C/I/V;
(36) the amino acid substitutions Q74L, S135G, I138T, R203G, and M218F, and optionally S153N;
(37) the amino acid substitutions Q74L/V/I/F/W/Y/C, I138T, S153N/C/I/V, R203G/Q and M218F/I/W/L, and optionally S135G/F;
(38) the amino acid substitutions Q74L, I138T, S153N, R203G, and M218F, and optionally S135G;
(39) the amino acid substitutions Q74L/V/I/F/W/Y/C, S135G/F, I138T, S153N/C/I/V, R203G/Q and M218F/I/W/L; or
(40) the amino acid substitutions Q74L, S135G, I138T, S153N, R203G and M218F.

Preferably, the polypeptide comprises in various embodiments
(1) the amino acid substitutions I138T, S153N/C/I/V and R203G/Q;
(2) the amino acid substitutions I138T, S153N and R203G;
(3) the amino acid substitutions Q74L/V/I/F/W/Y/C, I138T, S153N/C/I/V and R203G/Q;
(4) the amino acid substitutions Q74L, I138T, S153N and R203G;
(5) the amino acid substitutions S135G/F, I138T, S153N/C/I/V and R203G/Q;
(6) the amino acid substitutions S135G, I138T, S153N and R203G;
(7) the amino acid substitutions Q74L/V/I/F/W/Y/C, S135G/F, I138T, S153N/C/IN and R203G/Q;
(8) the amino acid substitutions Q74L, S135G, I138T, S153N and R203G;
(9) the amino acid substitutions Q74L/V/I/F/W/Y/C, S135G/F, I138T and R203G/Q;
(10) the amino acid substitutions Q74L, S135G, I138T and R203G;
(11) the amino acid substitutions Q74L/V/I/F/W/Y/C, S135G/F, I138T, S153N/C/I/V, R203G/Q and M218F/I/W/L; or
(12) the amino acid substitutions Q74L, S135G, I138T, S153N, R203G and M218F.

In another aspect, the polypeptide according to the invention comprises an amino acid sequence that has at least 80%, at least 85%, at least 90%, at least 95%, at least 97.5%, at least 99% or at least 99.5% sequence identity over its entire length with the amino acid sequence set forth in SEQ ID NO:1 and comprises the amino acid substitution S135G/F and I138T and optionally any one or more of Q74L/V/I/F/W/Y/C, S153N/C/IN, R203G/Q and M218F/I/W/L; wherein the amino acids at positions 68, 77, (scaffold), 46, 81 and 151 (catalytic triad) are invariable, and the amino acid at position 17 is S or A and the amino acid at position 219 is N/V/D/E/P/K (scaffold), wherein the positional numbering is according to SEQ ID NO:1, or a functional fragment thereof, wherein the polypeptide or the functional fragment has protease activity. In such an embodiment, the substitutions, if present, are preferably S135G, Q74L, S153N, R203G and/or M218F. All above specific embodiments in which S135G/F and I138T are both present, similarly apply to this embodiment and are preferred embodiments of this aspect. Specific examples are those that comprise all three of Q74LN/I/F/W/Y/C, S135G/F and I138T.

In various embodiments, the polypeptide of the invention further comprises any one or more of the amino acid substitution(s) L60W, F5C and L210C, wherein the positional numbering is according to SEQ ID NO:1.

The polypeptide can be an isolated polypeptide.

In various embodiments
(a) the polypeptide is up to 236 amino acids in length; and/or
(b) the polypeptide comprises or consists of the amino acid sequence set forth in any one of SEQ ID Nos. 13 to 17, 24, 27, 28 or 30.

In another aspect, the present invention pertains to a composition comprising the polypeptide of the invention.

In still another aspect, the invention is directed to a nucleic acid molecule encoding the polypeptide of the invention as well as vectors, such as plasmids, comprising a nucleic acid molecule of the invention.

The vector may be an expression vector and may comprise additional nucleic acid sequences necessary to facilitate its function in a host cell. The nucleic acid molecule may be an isolated nucleic acid molecule.

Host cells comprising a nucleic acid molecule or a vector according to the invention also form part of the invention. The host cell may be a prokaryotic host cell, for example an E. coli cell.

In another aspect, the invention is directed to a method for the production of a polypeptide as described herein, comprising
(1) cultivating the host cell described herein under conditions that allow the expression of the polypeptide; and
(2) isolating the expressed polypeptide from the host cell.

The method may, in various embodiments, further comprise recovering the expressed polypeptide from the host cell and/or the culture medium.

In still another aspect, the invention relates to a method for the cleavage of a substrate polypeptide comprising the amino acid sequence motif set forth in SEQ ID NO:18 (ENLYFQX), comprising contacting the substrate polypeptide with the polypeptide according to the invention under conditions that allow the cleavage of the polypeptide; and optionally purifying the cleaved polypeptide.

In a still further aspect, the invention is directed to the use of the polypeptide of the invention for cleavage of a substrate polypeptide comprising the amino acid motif of SEQ ID NO:18.

In various embodiments of the methods or uses of the invention, the substrate polypeptide is a fusion protein, preferably a non-natural fusion protein.

It is understood that all combinations of the above disclosed embodiments are also intended to fall within the scope of the present invention.

### BRIEF DESCRIPTION OF THE DRAWINGS

FIGS. 1A-1B show the rescreening activity results of 20 selected clones with Val peptide for Plate 1 (FIG. 1A) and Plate 2 (FIG. 1B).

FIG. 2 shows the rescreening results of 20 selected clones with both peptides. Dark grey bars: improvement over double mutant (in fold) for Val peptide. Light grey bars: Relative activity to the standard TEV with Gly peptide (SEQ ID NO:19). Dashed line—Standard TEV (SEQ ID NO:1)/DM (SEQ ID NO:3).

FIG. 3 shows the relative activity of some mutants before and after heat shock.

FIG. 4 shows the relative activity of some mutants with modified fluorogenic substrates, where the P-1 position was varied as indicated. 100% was the activity value in RFU of each variant with Gly, so each substrate was compared with variant's Gly activity levels.

FIG. 5 shows the relative activity of some mutants before and after heat shock including P1 rev. P1 repeat refers to a second expression of P1 in order to repeat the experiment as the same time as P1rev, obtaining the same result as before.

FIG. 6 shows an SDS-PAGE analysis of cell pellets 16 hours after protein expression induction.

FIGS. 7A-7B show an SDS-PAGE analysis (FIG. 7A) of cell lysates after cell lysis with BugBuster Kit and TEV protease activity assay (25 μM G and V fluorogenic substrates for 30 min, respectively, attention: Km values for DM and P1-M218F, see Table 3). Individual band intensities are shown in RFUs (relative fluorescence unit) (FIG. 7B). MBP: maltose binding protein.

FIG. 8 shows specific activities of DM, P1 and P1-M218F TEV proteases against 25 μM of all eight synthetic peptide substrates (attention: in some cases below or equal to Km values, see Table 3). The specific activities of all three TEV protease variants against 25 μM of synthetic peptide substrates were determined, respectively. Error bars indicate the standard deviation, measurements were performed in triplicates.

FIG. 9 shows the relative fluorescence unit (RFU) of cleavage reactions after 4 h of incubation (25 μM substrate).

FIG. 10 shows an SDS-PAGE analysis of cleavage reactions of DM, P1 and P1-M218F variants with 25 μM of Switchtag (HlyA fragment, 165 aa long)-Teriparatide (CAS: 52232-67-4, 34 aa long) substrates after 16 h of incubation (One grey box: Difference in cleavage efficiency observed for P1 and/or P1-M218F compared to DM. Two grey boxes: Difference in cleavage efficiency observed for P1 and P1-M218F. Protein bands of Switchtag-Teriparatide and Switchtag are indicated. Release of Teriparatide could not be detected due to limitations of the SDS-PAGE gel. Uncleaved Switchtag-Teriparatide substrates served as controls (C)).

FIGS. 11A-11B show the HPLC analysis of cleavage reactions of DM, P1 and P1-M218F with 25 μM of Switchtag-Teriparatide substrates after 1 h (FIG. 11A), 3 h (FIG. 11B), and 16 h (FIG. 11C) of incubation.

FIG. 12 shows the EDANS standard curve that is the average of duplicates prepared for each standard measurement.

FIG. 13 shows a sequence alignment of TEV-SM variant (SEQ ID NO:32) used in the experimental measurements and built model used in the computational study together with C1-model (SEQ ID NO:33). Modifications introduced to the original crystal TEV structure at both polypeptide ends are highlighted in dark grey. Furthermore, mutations distinguishing the SM variant from the wild type, the differences in the sequence between the SM and C1 models and the catalytic triad are highlighted.

FIG. 14 shows the determined pKa values for titratable residues present in the SM variant.

FIG. 15 shows the structure of the SM:substrate complex before (left panel) and after initial equilibration MD simulations (right panel). Added N- and C-termini fragments are highlighted, and the substrate backbone is shown. The positions of Cα atoms of residues of the catalytic triad are indicated by grey balls.

FIG. 16 shows the structure of the fluorogenic peptide substrate, SubG (ACE-E(EDANS)-NLYFQ-GG-K(DABCYL)) SEQ ID NO:38).

FIG. 17 shows the scheme of the active site with part of the fluorogenic peptide substrate, SubG (ACE-E(EDANS)-NLYFQ-GG-K(DABCYL)) (SEQ ID NO:38). The QM region is highlighted.

FIG. 18 shows the TEV protease substrate binding site. The TEV-protease with an example substrate is shown and the peptide P1′ position as well as protease residues T30, L32, H46, and the ²⁸HTTSLYGIG³⁶ beta sheet (SEQ ID NO: 43) and ²¹⁷FMSKP²²¹ loop (SEQ ID NO:44), which often sterically clash with P1′ when the Serine is computationally mutated, are shown. SenseNet and further analysis identified T22, D148, and M218 as final mutation candidates to improve P1′ tolerance.

FIG. 19 shows the structure of Switchtag-Teriparatide substrates.

FIG. 20 shows a SenseNet analysis of the TEV-protease-peptide interaction with P1′V. TEV-protease residues are shown as circles while the peptide with P1′V is shown as rectangles. Protease residues are colored from white to dark grey as their node correlation factor increases, and edges are depicted from thin to thick as their edge correlation factor increases. Solid lines indicate hydrophobic contacts, while dashed lines show H-bonds. Based on 234 ns unbiased MD simulation excluding the first 100 ns to focus on long-range effects occurring at later timescales. D148 and M218 show up as key residues for tolerance of P1′V (all encircled in bold).

FIGS. 21A-21B show library mutant screening for P1′V cleavage efficiency. FIG. 21A shows the mutations per clone, while FIG. 21B shows the relative cleavage activity of the tested clones over TEVp-SM for P1′G (second bar) or over TEVp-DM for P1′V (first bar). TEVp-DM was used for the P1′V comparison as TEVp-SM showed no activity against P1′V. Clone 1 shows the highest improvement for P1′V.

FIGS. 22A-22D show the purification of SM TEV protease variant. Purification of SM TEVp variant by (FIGS. 22A-22B) IMAC and (FIGS. 22C-22D) IEX chromatography. Shown are the (FIG. 22A) IMAC and (FIG. 22C) IEX chromatograms monitored at 280 nm and the coomassie-stained SDS-PAGE gels after (FIG. 22B) IMAC and (FIG. 22D) IEX purification. Cell lysate after cell disruption or IMAC load (L), flow through (FT), wash (W), pooled IMAC elution fractions (EP), molecular marker (M). The IMAC EP fractions were desalted (D) and purified by IEX chromatography. The IEX elution fraction 17 (E17) contains the purified SM TEVp variant.

FIGS. 23A-23D show the purification of clone 1 TEV protease variant. Purification of clone 1 TEVp variant by (FIGS. 23A-23B) IMAC and (FIGS. 23C-23D) IEX chromatography. Shown are the (FIG. 23A) IMAC and (FIG. 23C) IEX chromatograms monitored at 280 nm and the coomassie-stained SDS-PAGE gels after (FIG. 23B) IMAC and (FIG. 23D) IEX purification. Cell lysate after cell disruption or IMAC load (L), flow through (FT), wash (W), pooled IMAC elution fractions (EP), molecular marker (M). The IMAC EP fractions were desalted (D) and purified by IEX chromatography. The IEX elution fraction 16 (E16) contains the purified clone 1 TEVp variant.

FIG. 24 shows TEV protease activity against 25 μM of fluorogenic peptide substrates. The specific activities of SM and clone 1 TEV protease variants against 25 μM fluorogenic peptide substrates with different amino acids at the P1′ position (P1′: G, K, R, E, T, V, I and L). Error bars indicate the standard deviation, measurements were performed in triplicates.

FIG. 25 shows TEV protease activity against Switchtag-Teriparatide after 1 h incubation. Release of Teriparatide from Switchtag after proteolytic cleavage with SM and clone 1 TEVp for different P1′ residues, quantified by chromatographic elution peak integration (OpenLab ChemStation data software, Agilent). Shown are the relative cleavage efficiencies (in %) compared to SM TEVp with ENLYFQ-S (recognition/cleavage site) (set to 100%). Error bars indicate the standard deviation, measurements were performed in triplicates.

FIG. 26 shows an SDS-PAGE analysis of TEV protease activity against Switchtag-Teriparatide substrates after 1 and 16 h. Coomassie Brilliant Blue-stained SDS-PAGE gel analysis of cleavage reactions of Switchtag-Teriparatide substrates cleaved with SM and clone 1 TEVp variants after 1 and 16 hours incubation (panels A-P). Release of Switchtags after proteolytic cleavage are indicated. Switchtag-Teriparatide substrates in the absence of TEVp were incubated at 30° C. for 16 h and served as control (C). c1: clone 1 TEVp; SM: single mutant TEVp.

FIG. 27 shows an RP-HPLC-MS analysis of Teriparatide-Switchtag (recognition/cleavage site: ENLYFQ-S) cleavage reaction. Analytical-scale RP-HPLC-MS analysis of Switchtag-Teriparatide with the sequence ENLYFQ-S after TEVp cleavage and incubation at 30° C. for 3 h. Chromatograms show the UV absorption at 205 nm. Mass analysis of the elution signals were performed by ESI quadrupole mass spectrometry (ACQUITY QDa Detector, Waters) and confirmed the identity of Teriparatide in the elution peak at 17.073 min).

### DETAILED DESCRIPTION OF THE INVENTION

The terms used herein have, unless explicitly stated otherwise, the meanings as commonly understood in the art.

“At least one”, as used herein, relates to one or more, in particular 1, 2, 3, 4, 5, 6, 7, 8, 9, 10 or more.

“Isolated” as used herein in relation to a molecule means that said molecule has been at least partially separated from other molecules it naturally associates with or other cellular components. “Isolated” may mean that the molecule has been purified to separate it from other molecules and components, such as other proteins and nucleic acids and cellular debris.

“Nucleic acid” as used herein includes all natural forms of nucleic acids, such as DNA and RNA. Preferably, the nucleic acid molecules of the invention are DNA.

The term “peptide” is used throughout the specification to designate a polymer of amino acid residues connected to each other by peptide bonds. A peptide according to the present invention may have 2-100 amino acid residues. The terms “protein” and “polypeptide” are used interchangeably throughout the specification to designate a polymer of amino acid residues connected to each other by peptide bonds. A protein or polypeptide according to the present invention has preferably 100 or more amino acid residues.

The term “an N-terminal fragment” relates to a peptide or protein sequence which is in comparison to a reference peptide or protein sequence C-terminally truncated, such that a contiguous amino acid polymer starting from the N-terminus of the peptide or protein remains. In some embodiments, such fragments may have a length of at least 30 amino acids, at least 50 amino acids or at least 70 amino acids.

The term “a C-terminal fragment” relates to a peptide or protein sequence which is in comparison to a reference peptide or protein sequence N-terminally truncated, such that a contiguous amino acid polymer starting from the C-terminus of the peptide or protein remains. In some embodiments, such fragments may have a length of at least 30 amino acids, at least 50 amino acids or at least 70 amino acids.

The term “fusion protein” as used herein concerns two or more peptides and proteins which are N- or C-terminally connected to each other, typically by peptide bonds, including via an amino acid/peptide linker sequence. Such fusion proteins may be encoded by two or more nucleic acid sequences which are operably fused to each other. In certain embodiments, a fusion protein refers to at least one peptide or protein of interest C-terminally or N-terminally fused to the TEV variant amino acid sequence according to the invention, optionally via a linker sequence.

“Stability”, as used herein in relation to the polypeptides of the invention, primarily relates to resistance to (proteolytic) degradation and denaturation, which is a commonly encountered issue.

Generally, the skilled person understands that for putting the present invention into practice any nucleotide sequence described herein may comprise an additional start and/or stop codon or that a start and/or stop codon included in any of the sequences described herein may be deleted, depending on the nucleic acid construct used. The skilled person will base this decision, e.g., on whether a nucleic acid sequence comprised in the nucleic acid molecule of the present invention is to be translated and/or is to be translated as a fusion protein. In various embodiments, the polypeptides of the invention additionally comprise the amino acid M on the N-terminus of the polypeptide.

The present invention is based on the inventors finding that certain variants of TEV protease provide for an increased activity and altered substrate specificity as well as increased stability relative to TEV wildtype (SEQ ID NO:2) and known TEV variants (SEQ ID NO:1).

Thus, in a first aspect, the present invention relates to a(n) (isolated) polypeptide comprising an amino acid sequence that has at least 80%, at least 85%, at least 90%, at least 95%, at least 97.5%, at least 99% or at least 99.5% sequence identity over its entire length with the amino acid sequence set forth in SEQ ID NO:1 and comprises one or more amino acid substitution(s) selected from the group consisting of: M218F/W/L, T22V/I/S/Y, T30A/S, D148N/R/Y, Q74L/V/I/F/W/Y/C, and S135G/F, wherein the positions 46, 81 and 151 and, optionally, 17, 68, 77, and 219 are invariable, wherein the positional numbering is according to SEQ ID NO:1, or a functional fragment thereof. In various embodiments, the residues at positions 17 and 219 are only invariable insofar as they can be 17S/A and 219N/V/D/E/P/K.

In various embodiments, the polypeptide can additionally comprise one, two or all three of the substitutions: I138T, S153N/C/I/V and R203G/Q. In various embodiments, it is preferred that the polypeptides of the invention further comprise at least the R203G substitution. In such embodiments, the R203G substitution may then also be invariable. The substitution at position 218 may be 218F. The substitution at position 22 may be 22V/1. The substitution at position 30 may be 30A. The substitution at position 148 may be 148N/R. The substitution at position 74 may be 74L. The substitution at position 135 may be 135G. The substitution at position 153 may be 153N. The substitution at position 203 may be 203G. All these may be combined. In various embodiments, the one or more amino acid substitution(s) are thus selected from the group consisting of: M218F, T22V/I, T30A, D148N/R, Q74L, and S135G, wherein the positions 46, 81 and 151 and, optionally, 17, 68, 77, and 219 are invariable, wherein the positional numbering is according to SEQ ID NO:1, or a functional fragment thereof. In various embodiments, the residues at positions 17 and 219 are only invariable insofar as they can be 17S/A and 219N/V/D/E/P/K. In various such embodiments, the polypeptide can additionally comprise one, two or all three of the substitutions: I138T, S153N and R203G.

In another aspect, the polypeptide according to the invention comprises an amino acid sequence that has at least 80%, at least 85%, at least 90%, at least 95%, at least 97.5%, at least 99% or at least 99.5% sequence identity over its entire length with the amino acid sequence set forth in SEQ ID NO:1 and comprises the amino acid substitution Q74L/V/I/F/W/Y/C and optionally any one or more of S135G/F, I138T, S153N/C/I/V, R203G/Q and M218F/I/W/L; wherein the amino acids at positions 68, 77, (scaffold), 46, 81 and 151 (catalytic triad) are invariable, and the amino acid at position 17 is S or A and the amino acid at position 219 is N/V/D/E/P/K (scaffold), wherein the positional numbering is according to SEQ ID NO:1, or a functional fragment thereof, wherein the polypeptide or the functional fragment has protease activity. In such embodiments, the substitution at position 74 may be Q74L. In such embodiments, the substitution at position 135 may be 135G. In such embodiments, the substitution at position 153 may be 153N. In such embodiments, the substitution art position 203 may be 203G. In such embodiments, the substitution at position 218 may be 218F. All these are preferred and may be present in any combination.

In another aspect, the polypeptide according to the invention comprises an amino acid sequence that has at least 80%, at least 85%, at least 90%, at least 95%, at least 97.5%, at least 99% or at least 99.5% sequence identity over its entire length with the amino acid sequence set forth in SEQ ID NO:1 and comprises the amino acid substitution S135G/F and optionally any one or more of Q74L/V/I/F/W/Y/C, I138T, S153N/C/I/V, R203G/Q and M218F/I/W/L; wherein the amino acids at positions 68, 77, (scaffold), 46, 81 and 151 (catalytic triad) are invariable, and the amino acid at position 17 is S or A and the amino acid at position 219 is N/V/D/E/P/K (scaffold), wherein the positional numbering is according to SEQ ID NO:1, or a functional fragment thereof, wherein the polypeptide or the functional fragment has protease activity. In such embodiments, the substitution at position 74 may be Q74L. In such embodiments, the substitution at position 135 may be 135G. In such embodiments, the substitution at position 153 may be 153N. In such embodiments, the substitution art position 203 may be 203G. In such embodiments, the substitution at position 218 may be 218F. All these are preferred and may be present in any combination.

In another aspect, the polypeptide according to the invention comprises an amino acid sequence that has at least 80%, at least 85%, at least 90%, at least 95%, at least 97.5%, at least 99% or at least 99.5% sequence identity over its entire length with the amino acid sequence set forth in SEQ ID NO:1 and comprises the amino acid substitution I138T and optionally any one or more of Q74L/I/F/W/Y/C, S135G/F, S153N/C/I/V, R203G/Q and M218F/I/W/L; wherein the amino acids at positions 68, 77, (scaffold), 46, 81 and 151 (catalytic triad) are invariable, and the amino acid at position 17 is S or A and the amino acid at position 219 is N/V/D/E/P/K (scaffold), wherein the positional numbering is according to SEQ ID NO:1, or a functional fragment thereof, wherein the polypeptide or the functional fragment has protease activity. In such embodiments, the substitution at position 74 may be Q74L. In such embodiments, the substitution at position 135 may be 135G. In such embodiments, the substitution at position 153 may be 153N. In such embodiments, the substitution art position 203 may be 203G. In such embodiments, the substitution at position 218 may be 218F. All these are preferred and may be present in any combination.

The polypeptide does not include the amino acid sequence as set forth in SEQ ID NO:1 or SEQ ID NO:2, but comprises a variant thereof that comprises at least one substitution as defined above.

The amino acid sequence of the polypeptide of the invention has, over its entire length, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99% or 99.5% sequence identity with the corresponding part of the amino acid sequence set forth in SEQ ID NO:1. In various embodiments, the amino acid sequence is of the same length as the sequence set forth in SEQ ID NO:1. In other embodiments, it is a shortened fragment thereof that may be obtainable by deletions/truncations. Such truncated versions are also referred to herein as functional fragments and are further defined below.

In various embodiments, the sequence of the amino acid sequence with the exception of the above-indicated positions that may be substituted or are invariable, i.e. the remainder of the amino acid sequence that is not substituted or invariable, is essentially identical to the sequence set forth in SEQ ID NO:1, i.e. has sequence identities of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 97.5%, at least 98%, at least 98.5%, at least 99%, at least 99.5% or 100% with the sequence of SEQ ID NO:1.

Determination of the sequence identity of nucleic acid or amino acid sequences can be done by a sequence alignment based on well-established and commonly used BLAST algorithms (See, e.g. Altschul, S. F., Gish, W., Miller, W., Myers, E. W. & Lipman, D. J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, S.3389-3402). Such an alignment is based on aligning similar nucleotide or amino acid sequences stretches with each other. Another algorithm known in the art of said purpose is the FASTA algorithm. Alignments, in particular multiple sequence comparisons, are typically done by using computer programs. Commonly used are the Clustal series (See, e.g., Chenna et al. (2003): Multiple sequence alignment with the Clustal series of programs. Nucleic Acid Research 31, 3497-3500), T-Coffee (See, e.g., Notredame et al. (2000): T-Coffee: A novel method for multiple sequence alignments. J. Mol. Biol. 302, 205-217) or programs based on these known programs or algorithms.

Also possible are sequence alignments using the computer program Vector NTI® Suite 10.3 (Invitrogen Corporation, 1600 Faraday Avenue, Carlsbad, CA, USA) with the set standard parameters, with the AlignX module for sequence comparisons being based on the ClustalW. If not indicated otherwise, the sequence identity is determined using the BLAST algorithm.

Such a comparison also allows determination of the similarity of the compared sequences. Said similarity is typically expressed in percent identify, i.e. the portion of identical nucleotides/amino acids at the same or corresponding (in an alignment) sequence positions relative to the total number of the aligned nucleotides/amino acids. For example, if in an alignment 90 amino acids of a 100 aa long query sequence are identical to the amino acids in corresponding positions of a template sequence, the sequence identity is 90%. The broader term “homology” additionally considers conserved amino acid substitutions, i.e. amino acids that are similar in regard to their chemical properties, since those typically have similar chemical properties in a protein. Accordingly, such homology can be expressed in percent homology. If not indicated otherwise, sequence identity and sequence homology relate to the entire length of the aligned sequence.

In the context of the present invention, the feature that an amino acid position corresponds to a numerically defined position in SEQ ID NO:1 means that the respective position correlates to the numerically defined position in SEQ ID NO:1 in an alignment obtained as described above.

“Amino acid substitution”, as used herein, relates to modification of the sequence such that the amino acid residue occurring at the corresponding position in SEQ ID NO:1 is replaced by another amino acid residue. The amino acid residue for substitution is typically selected from the 20 proteinogenic amino acids G, A, V, L, I, F, C, M, P, R, K, H, N, Q, D, E, S, T, W, and Y. Accordingly, if a position in SEQ ID NO:1 is occupied by any one of these 20 amino acid residues, its substitution means that it is replaced by any one of the other 19 amino acids listed above. A “deletion” at one or more positions means that the residues adjacent to the deletion site are directly connected by a peptide bond.

The amino acids are typically referred to herein in the one-letter code. Furthermore, the nomenclature is used in line with the accepted meaning in the art, such that the starting amino acid is given first in one-letter code, followed by the positional number and then the target amino acid. If there are multiple options for the target amino acid, the individual options are separated by “/”. The indication “M218F” thus means that the methionine residue in position 218 or the position corresponding to position 218 is replaced by phenylalanine. The term “T22V/I” thus means that threonine in position 22 or a position corresponding to position 22 is replaced by either valine or isoleucine.

The amino acids in the positions that correspond to positions 46, 81 and 151 (of SEQ ID NO:1) are invariable, as these form the catalytic triad of the enzyme and are thus crucial for its proteolytic activity. These amino acids are typically 46H, 81 D and 151C and are generally conserved in the polypeptides of the invention. “Invariable”, as used herein, generally means that the respective amino acid at the indicated position may not be changed, i.e. substituted or deleted.

The polypeptides of the invention furthermore comprise, relative to TEV wildtype, substitutions in the positions that correspond to positions 17, 68, 77, 219 in SEQ ID NO:1. These are known substitutions that have beneficial effects on solubility and other enzymatic properties and are thus typically retained and invariable in the polypeptides of the invention. These substitutions that are to be retained and already reflected in the amino acid sequence of SEQ ID NO:1 are 17S, 68D, 77V and 219N. Relative to TEV wildtype, these substitutions are T17S, N68D, I77V and S219N. In various embodiments, position 17 can also be A, and position 219 can also be V, D, E, P or K. Insofar these two positions, 17 and 219 are indicated as being “invariable”, this term includes that they may also be exchanged for the indicated alternatives, i.e. 17A and 219 V/D/E/P/K. Relative to TEV wildtype, the substitutions are thus T17S/A, N68D, I77V and S219N/V/D/E/P/K. It may however be preferred that they are strictly invariable and thus remain 17S and 219N.

In various embodiments of the invention, the polypeptides of the invention thus have the invariable amino acids 46H, 81 D, 151C, T17S/A, N68D, I77V and S219N/V/D/E/P/K, preferably 46H, 81 D, 151C, 17S, 68D, 77V and 219N, with the positional numbering according to SEQ ID NO:1. Additionally, the polypeptides comprise one or more of the amino acid substitution(s) defined herein, such as those selected from the group consisting of:
(1) Q74L/V/I/F/W/Y/C, S135G/F, I138T, S153N/C/I/V, R203G/Q and M218F/I/W/L, preferably Q74L, S135G, I138T, S153N, R203G and M218F; or
(2) M218F/I/W/L, T22V/I, T30A, D148N/R, Q74L/V/I/F/W/Y/C, and S135G/F.

In various embodiments of the invention, the polypeptides of the invention have the invariable amino acids 46H, 81 D, 151C, 68D and 77V, and at position 17 S or A and at position 219 N, V, D, E, P or K, with the positional numbering according to SEQ ID NO:1, and additionally comprise one or more of the amino acid substitution(s) selected from the group consisting of: Q74L/V/I/F/W/Y/C, S135G/F, I138T, S153N/C/I/V, R203G/Q and M218F/I/W/L, preferably Q74L, S135G, I138T, S153N, R203G and M218F.

The amino acid sequence of the polypeptide of the present invention is, in various embodiments, up to 242 amino acids in length, preferably 202 to 236 or 202 to 219 amino acids in length. It is preferred that the polypeptide comprises the region that corresponds to amino acids 17 to 218 of SEQ ID NO:1, more preferably also any one or more of the amino acids that correspond to those at positions 219 to 236 and/or 1 to 16. The polypeptide of the invention may be even shorter and comprise only up to the amino acid at position 219, i.e. is a A220-242 TEV protease. It is further preferred that it also comprises the amino acids at position(s) 16, 15-16, 14-16, 13-16, 12-16, 11-16, 10-16, 9-16, 8-16, 7-16, 6-16, 5-16, 4-16, 3-16, 2-16 or 1-16, using the positional numbering of SEQ ID NO:1.

In various embodiments, the polypeptide of the invention is a A237-242 TEV protease, i.e. lacks relative to the TEV protease wildtype sequence (SEQ ID NO:2), the amino acids in positions 237-242, i.e. the C-terminus. Said deletion is already reflected in SEQ ID NO:1.

While the amino acid sequence may correspond to a continuous amino acid stretch of the amino acid sequence set forth in SEQ ID NO:1 having the indicated length, it is similarly possible that the amino acid sequence corresponds to discontinuous stretches of the amino acid sequence set forth in SEQ ID NO:1, for example if it corresponds to stretches of SEQ ID NO:1 with certain amino acids or amino acid sequences being deleted therefrom. The polypeptide may thus be derived from the amino acid sequence set forth in SEQ ID NO:1 by any one or more of an N-terminal truncation, a C-terminal truncation or a deletion of one or more amino acids, in particular as described above. Any such shortened variants of the amino acid sequence of SEQ ID NO:1 are covered by the term “fragment”, as used herein. The term “functional fragment” additionally implies that the respective polypeptide retains its catalytic activity, in particular, the polypeptide or its functional fragment has protease activity. It is preferred that the fragments of the invention have at least 50, 60, 70, 80, 90 or 100% of the catalytic activity of the wildtype TEV protease as set forth in SEQ ID NO:2 or, preferably, of the TEV protease variant having the amino acid sequence of SEQ ID NO:1 under identical conditions that allow for TEV protease activity.

In various embodiments, the polypeptides of the invention comprise one, two, three or more of the indicated amino acid substitutions. In all these and the following specific embodiments, the residues at positions 46, 81 and 151 are invariable. Similarly, the residues at positions 68 and 77 are also invariable and the residues at positions 17 and 219 are either invariable or may only be exchanged to 17A and 219V/D/E/P/K, respectively.

In various embodiments, the polypeptides comprise
(1) the amino acid substitution M218F and optionally any one or more of T22V/I, T30A, D148N/R, Q74L, and S135G;
(2) the amino acid substitution M218F and optionally any one or more of T22V/I, T30A, D148N/R, Q74L, S135G, I138T, S153N and R203G;
(3) the amino acid substitution M218F and any two, any three, any four, or all five of T22V/I, T30A, D148N/R, Q74L, and S135G;
(4) the amino acid substitution M218F and any two, any three, any four, any five, any six, any seven, or all 8 of T22V/I, T30A, D148N/R, Q74L, S135G, I138T, S153N and R203G;
(5) the amino acid substitutions M218F and T22V/I and optionally any one, any two, any three, or all four of T30A, D148N/R, Q74L, and S135G;
(6) the amino acid substitutions M218F and T22V/I and optionally any one, any two, any three, any four, any five, any six, or all seven of T30A, D148N/R, Q74L, S135G, I138T, S153N and R203G;
(7) the amino acid substitutions M218F and T30A and optionally any one, any two, any three, or all four of T22V/I, D148N/R, Q74L, and S135G;
(8) the amino acid substitutions M218F and T30A and optionally any one, any two, any three, any four, any five, any six, or all seven of T22V/I, D148N/R, Q74L, S135G, I138T, S153N and R203G;
(9) the amino acid substitutions M218F and D148N/R and optionally any one, any two, any three, or all four of T22V/I, T30A, Q74L, and S135G;
(10) the amino acid substitutions M218F and D148N/R and optionally any one, any two, any three, any four, any five, any six, or all seven of T22V/I, T30A, Q74L, S135G, I138T, S153N and R203G;
(11) the amino acid substitutions M218F and Q74L and optionally any one, any two, any three, or all four of T22V/I, T30A, D148N/R, and S135G;
(12) the amino acid substitutions M218F and Q74L and optionally any one, any two, any three, any four, any five, any six, or all seven of T22V/I, T30A, D148N/R, S135G, I138T, S153N and R203G;
(13) the amino acid substitutions M218F and S135G and optionally any one, any two, any three, or all four of T22V/I, T30A, D148N/R, and Q74L;
(14) the amino acid substitutions M218F and S135G and optionally any one, any two, any three, any four, any five, any six, or all seven of T22V/I, T30A, D148N/R, Q74L, I138T, S153N and R203G;
(15) the amino acid substitutions M218F and I138T and optionally any one, any two, any three, any four, any five, any six, or all seven of T22V/I, T30A, D148N/R, Q74L, S135G, S153N and R203G;
(16) the amino acid substitutions M218F and S153N and optionally any one, any two, any three, any four, any five, any six, or all seven of T22V/I, T30A, D148N/R, Q74L, S135G, I138T and R203G;
(17) the amino acid substitutions M218F and R203G and optionally any one, any two, any three, any four, any five, any six, or all seven of T22V/I, T30A, D148N/R, Q74L, S135G, I138T and S153N;
(18) the amino acid substitutions M218F, S153N and R203G and optionally any one, any two, any three, any four, any five, or all six of T22V/I, T30A, D148N/R, Q74L, S135G and I138T.

In various embodiments, the polypeptides of the invention comprise
(19) the amino acid substitution T22V/I and optionally any one or more of T30A, D148N/R, Q74L, S135G, and M218F;
(20) the amino acid substitution T22V/I and optionally any one or more of T30A, D148N/R, Q74L, S135G, I138T, S153N, R203G and M218F;
(21) the amino acid substitution T22V/I and any two, any three, any four, or all five of T30A, D148N/R, Q74L, S135G, and M218F;
(22) the amino acid substitution T22V/I and any two, any three, any four, any five, any six, any seven, or all eight of T30A, D148N/R, Q74L, S135G, I138T, S153N, R203G, and M218F;
(23) the amino acid substitutions T22V/I and T30A and optionally any one, any two, any three, r all four of M218F, D148N/R, Q74L, and S135G;
(24) the amino acid substitution T22V/I and T30A and optionally any one, any two, any three, any four, any five, any six, or all seven of M218F, D148N/R, Q74L, S135G, I138T, S153N and R203G;
(25) the amino acid substitutions T22V/I and D148N/R and optionally any one, any two, any three, or all four of M218F, T30A, Q74L, and S135G;
(26) the amino acid substitution T22V/I and D148N/R and optionally any one, any two, any three, any four, any five, any six, or all seven of M218F, T30A, Q74L, S135G, I138T, S153N and R203G;
(27) the amino acid substitutions T22V/I and Q74L and optionally any one, any two, any three, or all four of M218F, T30A, D148N/R, and S135G;
(28) the amino acid substitutions T22V/I and Q74L and optionally any one, any two, any three, any four, any five, any six, or all seven of M218F, T30A, D148N/R, S135G, I138T, S153N and R203G;
(29) the amino acid substitutions T22V/I and S135G and optionally any one, any two, any three, or all four of M218F, T30A, D148N/R, and Q74L;
(30) the amino acid substitutions T22V/I and S135G and optionally any one, any two, any three, any four, any five, any six, or all seven of M218F, T30A, D148N/R, Q74L, I138T, S153N and R203G;
(31) the amino acid substitutions T22V/I and I138T and optionally any one, any two, any three, any four, any five, any six, or all seven of M218F, T30A, D148N/R, Q74L, S135G, S153N and R203G;
(32) the amino acid substitutions T22V/I and S153N and optionally any one, any two, any three, any four, any five, any six, or all seven of M218F, T30A, D148N/R, Q74L, S135G, I138T and R203G;
(33) the amino acid substitutions T22V/I and R203G and optionally any one, any two, any three, any four, any five, any six, or all seven of M218F, T30A, D148N/R, Q74L, S135G, I138T and S153N;
(34) the amino acid substitutions T22V/I, S153N and R203G and optionally any one, any two, any three, any four, any five, or all six of M218F, T30A, D148N/R, Q74L, S135G and I138T.

In various embodiments, the polypeptides of the invention comprise
(35) the amino acid substitution T30A and optionally any one or more of T22V/I, D148N/R, Q74L, S135G, and M218F;
(36) the amino acid substitution T30A and optionally any one or more of T22V/I, D148N/R, Q74L, S135G, I138T, S153N, R203G and M218F;
(37) the amino acid substitution T30A and any two, any three, any four, or all five of T22V/I, D148N/R, Q74L, S135G, and M218F;
(38) the amino acid substitution T30A and any two, any three, any four, any five, any six, any seven, or all eight of T22V/I, D148N/R, Q74L, S135G, I138T, S153N, R203G, and M218F;
(39) the amino acid substitutions T30A and D148N/R and optionally any one, any two, any three, or all four of M218F, T22V/I, Q74L, and S135G;
(40) the amino acid substitution T30A and D148N/R and optionally any one, any two, any three, any four, any five, any six, or all seven of M218F, T22V/I, Q74L, S135G, I138T, S153N and R203G;
(41) the amino acid substitutions T30A and Q74L and optionally any one, any two, any three, or all four of M218F, T22V/I, D148N/R, and S135G; (42) the amino acid substitutions T30A and Q74L and optionally any one, any two, any three, any four, any five, any six, or all seven of M218F, T22V/I, D148N/R, S135G, I138T, S153N and R203G;
(43) the amino acid substitutions T30A and S135G and optionally any one, any two, any three, or all four of M218F, T22V/I, D148N/R, and Q74L;
(44) the amino acid substitutions T30A and S135G and optionally any one, any two, any three, any four, any five, any six, or all seven of M218F, T22V/I, D148N/R, Q74L, I138T, S153N and R203G;
(45) the amino acid substitutions T30A and I138T and optionally any one, any two, any three, any four, any five, any six, or all seven of M218F, T22V/I, D148N/R, Q74L, S135G, S153N and R203G;
(46) the amino acid substitutions T30A and S153N and optionally any one, any two, any three, any four, any five, any six, or all seven of M218F, T22V/I, D148N/R, Q74L, S135G, I138T and R203G;
(47) the amino acid substitutions T30A and R203G and optionally any one, any two, any three, any four, any five, any six, or all seven of M218F, T22V/I, D148N/R, Q74L, S135G, I138T and S153N;
(48) the amino acid substitutions T30A, S153N and R203G and optionally any one, any two, any three, any four, any five, or all six of M218F, T22V/I, D148N/R, Q74L, S135G and I138T.

In various embodiments of the invention, the polypeptide comprises
(49) the amino acid substitution D148N/R and optionally any one or more of T22V/I, T30A, Q74L, S135G, and M218F;
(50) the amino acid substitution D148N/R and optionally any one or more of T22V/I, T30A, Q74L, S135G, I138T, S153N, R203G and M218F;
(51) the amino acid substitution D148N/R and any two, any three, any four, or all five of T22V/I, T30A, Q74L, S135G, and M218F;
(52) the amino acid substitution D148N/R and any two, any three, any four, any five, any six, any seven, or all eight of T22V/I, T30A, Q74L, S135G, I138T, S153N, R203G, and M218F;
(53) the amino acid substitutions D148N/R and Q74L and optionally any one, any two, any three, or all four of M218F, T22V/I, T30A, and S135G;
(54) the amino acid substitutions D148N/R and Q74L and optionally any one, any two, any three, any four, any five, any six, or all seven of M218F, T22V/I, T30A, S135G, I138T, S153N and R203G;
(55) the amino acid substitutions D148N/R and S135G and optionally any one, any two, any three, or all four of M218F, T22V/I, T30A, and Q74L;
(56) the amino acid substitutions D148N/R and S135G and optionally any one, any two, any three, any four, any five, any six, or all seven of M218F, T22V/I, T30A, Q74L, I138T, S153N and R203G;
(57) the amino acid substitutions D148N/R and I138T and optionally any one, any two, any three, any four, any five, any six, or all seven of M218F, T22V/I, T30A, Q74L, S135G, S153N and R203G;
(58) the amino acid substitutions D148N/R and S153N and optionally any one, any two, any three, any four, any five, any six, or all seven of M218F, T22V/I, T30A, Q74L, S135G, I138T and R203G;
(59) the amino acid substitutions D148N/R and R203G and optionally any one, any two, any three, any four, any five, any six, or all seven of M218F, T22V/I, T30A, Q74L, S135G, I138T and S153N;
(60) the amino acid substitutions D148N/R, S153N and R203G and optionally any one, any two, any three, any four, any five, or all six of M218F, T22V/I, T30A, Q74L, S135G and I138T.

In various embodiments, the polypeptide of the invention comprises
(61) the amino acid substitution Q74L and optionally any one or more of T22V/I, T30A, D148N/R, S135G, and M218F;
(62) the amino acid substitution Q74L and optionally any one or more of T22V/I, T30A, D148N/R, S135G, I138T, S153N, R203G and M218F;
(63) the amino acid substitution Q74L and any two, any three, any four, or all five of T22V/I, T30A, D148N/R, S135G, and M218F;
(64) the amino acid substitution Q74L and any two, any three, any four, any five, any six, any seven, or all eight of T22V/I, T30A, D148N/R, S135G, I138T, S153N, R203G, and M218F;
(65) the amino acid substitutions Q74L and S135G and optionally any one, any two, any three, or all four of M218F, T22V/I, T30A, and D148N/R;
(66) the amino acid substitutions Q74L and S135G and optionally any one, any two, any three, any four, any five, any six, or all seven of M218F, T22V/I, T30A, D148N/R, I138T, S153N and R203G;
(67) the amino acid substitutions Q74L and I138T and optionally any one, any two, any three, any four, any five, any six, or all seven of M218F, T22V/I, T30A, D148N/R, S135G, S153N and R203G;
(68) the amino acid substitutions Q74L and S153N and optionally any one, any two, any three, any four, any five, any six, or all seven of M218F, T22V/I, T30A, D148N/R, S135G, I138T and R203G;
(69) the amino acid substitutions Q74L and R203G and optionally any one, any two, any three, any four, any five, any six, or all seven of M218F, T22V/I, T30A, D148N/R, S135G, I138T and S153N;
(70) the amino acid substitutions Q74L, S153N and R203G and optionally any one, any two, any three, any four, any five, or all six of M218F, T22V/I, T30A, D148N/R, S135G and I138T.

In various embodiments, the polypeptide of the invention comprises
(71) the amino acid substitution S135G and optionally any one or more of T22V/I, T30A, D148N/R, Q74L, and M218F;
(72) the amino acid substitution S135G and optionally any one or more of T22V/I, T30A, D148N/R, Q74L, I138T, S153N, R203G and M218F;
(73) the amino acid substitution S135G and any two, any three, any four, or all five of T22V/I, T30A, D148N/R, Q74L, and M218F;
(74) the amino acid substitution S135G and any two, any three, any four, any five, any six, any seven, or all eight of T22V/I, T30A, D148N/R, Q74L, I138T, S153N, R203G, and M218F;
(75) the amino acid substitutions S135G and I138T and optionally any one, any two, any three, any four, any five, any six, or all seven of M218F, T22V/I, T30A, D148N/R, Q74L, S153N and R203G;
(76) the amino acid substitutions S135G and S153N and optionally any one, any two, any three, any four, any five, any six, or all seven of M218F, T22V/I, T30A, D148N/R, Q74L, I138T and R203G;
(77) the amino acid substitutions S135G and R203G and optionally any one, any two, any three, any four, any five, any six, or all seven of M218F, T22V/I, T30A, D148N/R, Q74L, I138T and S153N;
(78) the amino acid substitutions S135G, S153N and R203G and optionally any one, any two, any three, any four, any five, or all six of M218F, T22V/I, T30A, D148N/R, Q74L and I138T.

In various embodiments, the polypeptide comprises at least three of the indicated substitutions. In some embodiments, such polypeptides comprise
(79) the amino acid substitutions M218F, T22V/I and T30A and any one, any two, or all three of D148N/R, Q74L, and S135G;
(80) the amino acid substitutions M218F, T22V/I and D148N/R and any one, any two, or all three of T30A, Q74L, and S135G;
(81) the amino acid substitutions M218F, T30A and D148N/R and any one, any two, or all three of T22V/I, Q74L, and S135G;
(82) the amino acid substitutions T22V/I, T30A and D148N/R and any one, any two, or all three of M218F, Q74L, and S135G;
(83) the amino acid substitutions M218F, T22V/I, T30A and D148N/R and any one, or both of Q74L and S135G;
(84) the amino acid substitutions M218F, T22V/I, T30A and D148N/R and any one, any two, any three, any four, or all five of Q74L, S135G, I138T, S153N, and R203G.

In all embodiments disclosed herein, T22V/I may be T22V. Alternatively, in all embodiments disclosed herein T22V/I may be T22I. In some embodiments, T22V may be preferred over T22I.

In all embodiments disclosed herein, D148N/R may be D148N. Alternatively, in all embodiments disclosed herein D148N/R may be D148R.

In various other embodiments, the polypeptide may comprise
(1) the amino acid substitutions Q74L/V/I/F/W/Y/C and S135G/F, and optionally any one or more of I138T, S153N/C/I/V, R203G/Q and M218F/I/W/L;
(2) the amino acid substitutions Q74L and S135G, and optionally any one or more of I138T, S153N, R203G and M218F;
(3) the amino acid substitutions Q74L/V/I/F/W/Y/C and I138T, and optionally any one or more of S135G/F, S153N/C/I/V, R203G/Q and M218F/I/W/L;
(4) the amino acid substitutions Q74L and I138T, and optionally any one or more of S135G, S153N, R203G and M218F;
(5) the amino acid substitutions Q74L/V/I/F/W/Y/C and S153N/C/I/V, and optionally any one or more of S135G/F, I138T, R203G/Q and M218F/I/W/L;
(6) the amino acid substitutions Q74L, and S153N, and optionally any one or more of S135G, I138T, R203G and M218F;
(7) the amino acid substitutions Q74L/V/I/F/W/Y/C and R203G/Q, and optionally any one or more of S135G/F, I138T, S153N/C/I/V and M218F/I/W/L;
(8) the amino acid substitutions Q74L, and R203G, and optionally any one or more of S135G, I138T, S153N and M218F;
(9) the amino acid substitutions Q74L/V/I/F/W/Y/C and M218F/I/W/L, and optionally any one or more of S135G/F, I138T, S153N/C/I/V and R203G/Q;
(10) the amino acid substitutions Q74L, and M218F, and optionally any one or more of S135G, I138T, S153N and R203G;
(11) the amino acid substitutions Q74L/V/I/F/W/Y/C, S135G/F and I138T, and optionally any one, any two or three of S153N/C/I/V, R203G/Q and M218F/I/W/L;
(12) the amino acid substitutions Q74L, S135G and I138T, and optionally any one, any two or three of S153N, R203G and M218F;
(13) the amino acid substitutions Q74L/V/I/F/W/Y/C, S135G/F and S153N/C/I/V, and optionally any one, any two or three of I138T, R203G/Q and M218F/I/W/L;
(14) the amino acid substitutions Q74L, S135G, and S153N, and optionally any one, any two or three of I138T, R203G and M218F;
(15) the amino acid substitutions Q74L/V/I/F/W/Y/C, S135G/F and R203G/Q, and optionally any one, any two or three of I138T, S153N/C/I/V and M218F/I/W/L;
(16) the amino acid substitutions Q74L, S135G, and R203G, and optionally any one, any two or three of I138T, S153N and M218F;
(17) the amino acid substitutions Q74L/V/I/F/W/Y/C, S135G/F and M218F/I/W/L, and optionally any one, any two or three of I138T, S153N/C/I/V and R203G/Q;
(18) the amino acid substitutions Q74L, S135G, and M218F, and optionally any one, any two or three of I138T, S153N and R203G;
(19) the amino acid substitutions Q74L/V/I/F/W/Y/C, I138T and S153N/C/I/V, and optionally any one, any two or three of S135G/F, R203G/Q and M218F/I/W/L;
(20) the amino acid substitutions Q74L, I138T and S153N, and optionally any one, any two or three of S135G, R203G and M218F;
(21) the amino acid substitutions Q74L/V/I/F/W/Y/C, I138T and R203G/Q, and optionally any one, any two or three of S135G/F, S153N/C/I/V and M218F/I/W/L;
(22) the amino acid substitutions Q74L, I138T and R203G, and optionally any one, any two or three of S135G, S153N and M218F;
(23) the amino acid substitutions Q74L/V/I/F/W/Y/C, I138T and M218F/I/W/L, and optionally any one, any two or three of S135G/F, S153N/C/I/V and R203G/Q;
(24) the amino acid substitutions Q74L, I138T and M218F, and optionally any one, any two or three of S135G, S153N and R203G;
(25) the amino acid substitutions Q74L/V/I/F/W/Y/C, S153N/C/I/V and R203G/Q, and optionally any one, any two or three of S135G/F, I138T, M218F/I/W/L;
(26) the amino acid substitutions Q74L, S153N, and R203G, and optionally any one, any two or three of S135G, I138T and M218F;
(27) the amino acid substitutions Q74L/V/I/F/W/Y/C, S153N/C/I/V and M218F/I/W/L, optionally any one, any two or three of S135G/F, I138T, R203G/Q;
(28) the amino acid substitutions Q74L, S153N, and M218F, and optionally any one, any two or three of S135G, I138T and R203G;
(29) the amino acid substitutions Q74L/V/I/F/W/Y/C, R203G/Q and M218F/I/W/L, and optionally any one, any two or three of S135G/F, I138T, S153N/C/I/V;
(30) the amino acid substitutions Q74L, R203G, and M218F, and optionally any one, any two or three of S135G, I138T and S153N;
(31) the amino acid substitutions Q74L/V/I/F/W/Y/C, S135G/F, I138T and S153N/C/IN, and optionally any one or two of R203G/Q and M218F/I/W/L;
(32) the amino acid substitutions Q74L, S135G, I138T and S153N, and optionally any one or two of R203G and M218F;
(33) the amino acid substitutions Q74L/V/I/F/W/Y/C, S135G/F, I138T and R203G/Q, and optionally any one or two of S153N/C and M218F/I/W/L;
(34) the amino acid substitutions Q74L, S135G, I138T and R203G, and optionally any one or two of S153N and M218F;
(35) the amino acid substitutions Q74L/V/I/F/W/Y/C, S135G/F, I138T and M218F/I/W/L, and optionally any one or two of S153N/C/IN and R203G/Q;
(36) the amino acid substitutions Q74L, S135G, I138T and M218F, and optionally any one or two of S153N and R203G;
(37) the amino acid substitutions Q74L/V/I/F/W/Y/C, S135G/F, S153N/C/I/V, and R203G/Q, and optionally any one or two of I138T and M218F/I/W/L;
(38) the amino acid substitutions Q74L, S135G, S153N and R203G, and optionally any one or two of I138T and M218F;
(39) the amino acid substitutions Q74L/V/I/F/W/Y/C, S135G/F, S153N/C/I/V and M218F/I/W/L, and optionally any one or two of I138T and R203G/Q;
(40) the amino acid substitutions Q74L, S135G, S153N and M218F, and optionally any one or two of I138T and R203G;
(41) the amino acid substitutions Q74L/V/I/F/W/Y/C, I138T, S153N/C/I/V and R203G/Q, and optionally any one or two of S135G/F and M218F/I/W/L;
(42) the amino acid substitutions Q74L, I138T, S153N, and R203G, and optionally any one or two of S135G and M218F;
(43) the amino acid substitutions Q74L/V/I/F/W/Y/C, I138T, S153N/C/I/V and M218F/I/W/L, and optionally any one or two of S135G/F and R203G/Q;
(44) the amino acid substitutions Q74L, I138T, S153N, and M218F, and optionally any one or two of S135G and R203G;
(45) the amino acid substitutions Q74L/V/I/F/W/Y/C, S153N/C, R203G/Q and M218F/I/W/L, and optionally any one or two of S135G/F and I138T;
(46) the amino acid substitutions Q74L, S153N, R203G, and M218F, and optionally any one or two of S135G and I138T;
(47) the amino acid substitutions Q74L/V/I/F/W/Y/C, S135G/F, R203G/Q, and M218F/I/W/L, and optionally any one or two of I138T and S153N/C/I/V;
(48) the amino acid substitutions Q74L, S135G, R203G, and M218F, and optionally any one or two of I138T and S153N;
(49) the amino acid substitutions Q74L/V/I/F/W/Y/C, S135G/F, I138T, S153N/C/I/V and R203G/Q, and optionally M218F/I/W/L;
(50) the amino acid substitutions Q74L, S135G, I138T, S153N, and R203G, and optionally M218F;
(51) the amino acid substitutions Q74L/V/I/F/W/Y/C, S135G/F, I138T, S153N/C/I/V and M218F/I/W/L, and optionally R203G/Q;
(52) the amino acid substitutions Q74L, S135G, I138T, S153N, and M218F, and optionally R203G;
(53) the amino acid substitutions Q74L/V/I/F/W/Y/C, S135G/F, I138T, R203G/Q and M218F/I/W/L, and optionally S153N/C/I/V;
(54) the amino acid substitutions Q74L, S135G, I138T, R203G, and M218F, and optionally S153N;
(55) the amino acid substitutions Q74L/V/I/F/W/Y/C, S135G/F, S153N/C/I/V, R203G/Q and M218F/I/W/L, and optionally I138T;
(56) the amino acid substitutions Q74L, S135G, S153N, R203G, and M218F, and optionally I138T;
(57) the amino acid substitutions Q74L/V/I/F/W/Y/C, I138T, S153N/C/I/V, R203G/Q and M218F/I/W/L, and optionally S135G/F;
(58) the amino acid substitutions Q74L, I138T, S153N, R203G and M218F, and optionally S135G;
(59) the amino acid substitutions Q74L/V/I/F/W/Y/C, S135G/F, I138T, S153N/C/I/V, R203G/Q and M218F/I/W/L; or
(60) the amino acid substitutions Q74L, S135G, I138T, S153N, R203G and M218F.

Alternatively, the polypeptide may comprise, in various embodiments,
(1) the amino acid substitutions S135G/F and I138T, and optionally any one or more of Q74L/V/I/F/W/Y/C, S153N/C/I/V, R203G/Q and M218F/I/W/L;
(2) the amino acid substitutions I138T and S135G, and optionally any one or more of Q74L, S153N, R203G and M218F;
(3) the amino acid substitutions S135G/F and S153N/C/I/V, and optionally any one or more of Q74L/V/I/F/W/Y/C, I138T, R203G/Q and M218F/I/W/L;
(4) the amino acid substitutions S135G and S153N, and optionally any one or more of Q74L, I138T, R203G and M218F;
(5) the amino acid substitutions S135G/F and R203G/Q, and optionally any one or more of Q74L/V/I/F/W/Y/C, I138T, S153N/C/I/V and M218F/I/W/L;
(6) the amino acid substitutions S135G and R203G, and optionally any one or more of Q74L, I138T, S153N and M218F;
(7) the amino acid substitutions S135G/F and M218F/I/W/L, and optionally any one or more of Q74L/V/I/F/W/Y/C, I138T, S153N/C/I/V and R203G/Q;
(8) the amino acid substitutions S135G and M218F, and optionally any one or more of Q74L, I138T, S153N and R203G;
(9) the amino acid substitutions Q74L/V/I/F/W/Y/C, S135G/F and I138T, and optionally any one, any two or three of S153N/C/I/V, R203G/Q and M218F/I/W/L;
(10) the amino acid substitutions Q74L, S135G and I138T, and optionally any one, any two or three of S153N, R203G and M218F;
(11) the amino acid substitutions I138T, S135G/F and S153N/C/I/V, and optionally any one, any two or three of Q74L/V/I/F/W/Y/C, R203G/Q and M218F/I/W/L;
(12) the amino acid substitutions I138T, S135G, and S153N, and optionally any one, any two or three of Q74L, R203G and M218F;
(13) the amino acid substitutions I138T, S135G/F and R203G/Q, and optionally any one, any two or three of Q74L/V/I/F/W/Y/C, S153N/C/I/V and M218F/I/W/L;
(14) the amino acid substitutions I138T, S135G, and R203G, and optionally any one, any two or three of Q74L, S153N and M218F;
(15) the amino acid substitutions I138T, S135G/F and M218F/I/W/L, and optionally any one, any two or three of Q74L/V/I/F/W/Y/C, S153N/C/I/V and R203G/Q;
(18) the amino acid substitutions I138T, S135G, and M218F, and optionally any one, any two or three of Q74L, S153N and R203G;
(19) the amino acid substitutions S135G/F, I138T, S153N/C/I/V, and R203G/Q, and optionally any one or two of Q74L/V/I/F/W/Y/C and M218F/I/W/L;
(20) the amino acid substitutions S135G, I138T, S153N and R203G, and optionally any one or two of Q74L and M218F;
(21) the amino acid substitutions S135G/F, I138T, S153N/C/I/V and M218F/I/W/L, and optionally any one or two of Q74L/I/F/W/Y/C and R203G/Q;
(22) the amino acid substitutions S135G, I138T, S153N and M218F, and optionally any one or two of Q74L and R203G;
(23) the amino acid substitutions S135G/F, I138T, S153N/C/I/V and R203G/Q, and optionally any one or two of Q74L/V/I/F/W/Y/C and M218F/I/W/L;
(24) the amino acid substitutions S135G, I138T, S153N, and R203G, and optionally any one or two of Q74L and M218F;
(25) the amino acid substitutions S135G/F, I138T, S153N/C/I/V and M218F/I/W/L, and optionally any one or two of Q74L/V/I/F/W/Y/C and R203G/Q;
(26) the amino acid substitutions S135G, I138T, S153N, and M218F, and optionally any one or two of Q74L and R203G;
(27) the amino acid substitutions S135G/F, S153N/C, R203G/Q and M218F/I/W/L, and optionally any one or two of Q74L/V/I/F/W/Y/C and I138T;
(28) the amino acid substitutions S135G, S153N, R203G, and M218F, and optionally any one or two of Q74L and I138T;
(29) the amino acid substitutions I138T, S135G/F, R203G/Q, and M218F/I/W/L, and optionally any one or two of Q74L/V/I/F/W/Y/C and S153N/C/I/V;
(30) the amino acid substitutions I138T, S135G, R203G, and M218F, and optionally any one or two of Q74L and S153N;
(31) the amino acid substitutions Q74L/V/I/F/W/Y/C, S135G/F, I138T, S153N/C/I/V and R203G/Q, and optionally M218F/I/W/L;
(32) the amino acid substitutions Q74L, S135G, I138T, S153N, and R203G, and optionally M218F;
(33) the amino acid substitutions Q74L/V/I/F/W/Y/C, S135G/F, I138T, S153N/C/I/V and M218F/I/W/L, and optionally R203G/Q;
(34) the amino acid substitutions Q74L, S135G, I138T, S153N, and M218F, and optionally R203G;
(35) the amino acid substitutions Q74L/V/I/F/W/Y/C, S135G/F, I138T, R203G/Q and M218F/I/W/L, and optionally S153N/C/I/V;
(36) the amino acid substitutions Q74L, S135G, I138T, R203G, and M218F, and optionally S153N;
(37) the amino acid substitutions Q74L/V/I/F/W/Y/C, S135G/F, S153N/C/I/V, R203G/Q and M218F/I/W/L, and optionally I138T;
(38) the amino acid substitutions Q74L, S135G, S153N, R203G, and M218F, and optionally I138T;
(39) the amino acid substitutions Q74L/V/I/F/W/Y/C, S135G/F, I138T, S153N/C/I/V, R203G/Q and M218F/I/W/L; or
(40) the amino acid substitutions Q74L, S135G, I138T, S153N, R203G and M218F.

Alternatively, the polypeptide may comprise, in various embodiments,
(1) the amino acid substitutions S135G/F and I138T, and optionally any one or more of Q74L/V/I/F/W/Y/C, S153N/C/I/V, R203G/Q and M218F/I/W/L;
(2) the amino acid substitutions I138T and S135G, and optionally any one or more of Q74L, S153N, R203G and M218F;
(3) the amino acid substitutions I138T and S153N/C/I/V, and optionally any one or more of Q74L/V/I/F/W/Y/C, S135G/F, R203G/Q and M218F/I/W/L;
(4) the amino acid substitutions I1b38T and S153N, and optionally any one or more of Q74L, S135G, R203G and M218F;
(5) the amino acid substitutions I138T and R203G/Q, and optionally any one or more of Q74L/V/I/F/W/Y/C, S135G/F, S153N/C/I/V and M218F/I/W/L;
(6) the amino acid substitutions I138T and R203G, and optionally any one or more of Q74L, S135G, S153N and M218F;
(7) the amino acid substitutions I138T and M218F/I/W/L, and optionally any one or more of Q74L/V/I/F/W/Y/C, S135G/F, S153N/C/I/V and R203G/Q;
(8) the amino acid substitutions I138T and M218F, and optionally any one or more of Q74L, S135G, S153N and R203G;
(9) the amino acid substitutions Q74LN/I/F/W/Y/C, S135G/F and I138T, and optionally any one, any two or three of S153N/C/I/V, R203G/Q and M218F/I/W/L;
(10) the amino acid substitutions Q74L, S135G and I138T, and optionally any one, any two or three of S153N, R203G and M218F;
(11) the amino acid substitutions I138T, S135G/F and S153N/C/I/V, and optionally any one, any two or three of Q74L/V/I/F/W/Y/C, R203G/Q and M218F/I/W/L;
(12) the amino acid substitutions I138T, S135G, and S153N, and optionally any one, any two or three of Q74L, R203G and M218F;
(13) the amino acid substitutions I138T, S135G/F and R203G/Q, and optionally any one, any two or three of Q74L/V/I/F/W/Y/C, S153N/C/I/V and M218F/I/W/L;
(14) the amino acid substitutions I138T, S135G, and R203G, and optionally any one, any two or three of Q74L, S153N and M218F;
(15) the amino acid substitutions I138T, S135G/F and M218F/I/W/L, and optionally any one, any two or three of Q74L/V/I/F/W/Y/C, S153N/C/I/V and R203G/Q;
(18) the amino acid substitutions I138T, S135G, and M218F, and optionally any one, any two or three of Q74L, S153N and R203G;
(19) the amino acid substitutions S135G/F, I138T, S153N/C/I/V, and R203G/Q, and optionally any one or two of Q74L/V/I/F/W/Y/C and M218F/I/W/L;
(20) the amino acid substitutions S135G, I138T, S153N and R203G, and optionally any one or two of Q74L and M218F;
(21) the amino acid substitutions S135G/F, I138T, S153N/C/I/V and M218F/I/W/L, and optionally any one or two of Q74L/I/F/W/Y/C and R203G/Q;
(22) the amino acid substitutions S135G, I138T, S153N and M218F, and optionally any one or two of Q74L and R203G;
(23) the amino acid substitutions S135G/F, I138T, S153N/C/I/V and R203G/Q, and optionally any one or two of Q74L/V/I/F/W/Y/C and M218F/I/W/L;
(24) the amino acid substitutions S135G, I138T, S153N, and R203G, and optionally any one or two of Q74L and M218F;
(25) the amino acid substitutions S135G/F, I138T, S153N/C/I/V and M218F/I/W/L, and optionally any one or two of Q74L/V/I/F/W/Y/C and R203G/Q;
(26) the amino acid substitutions S135G, I138T, S153N, and M218F, and optionally any one or two of Q74L and R203G;
(27) the amino acid substitutions I138T, S153N/C, R203G/Q and M218F/I/W/L, and optionally any one or two of Q74L/V/I/F/W/Y/C and S135G/F;
(28) the amino acid substitutions I138T, S153N, R203G, and M218F, and optionally any one or two of Q74L and S135G;
(29) the amino acid substitutions I138T, S135G/F, R203G/Q, and M218F/I/W/L, and optionally any one or two of Q74L/V/I/F/W/Y/C and S153N/C/I/V;
(30) the amino acid substitutions I138T, S135G, R203G, and M218F, and optionally any one or two of Q74L and S153N;
(31) the amino acid substitutions Q74L/V/I/F/W/Y/C, S135G/F, I138T, S153N/C/I/V and R203G/Q, and optionally M218F/I/W/L;
(32) the amino acid substitutions Q74L, S135G, I138T, S153N, and R203G, and optionally M218F;
(33) the amino acid substitutions Q74L/V/I/F/W/Y/C, S135G/F, I138T, S153N/C/I/V and M218F/I/W/L, and optionally R203G/Q;
(34) the amino acid substitutions Q74L, S135G, I138T, S153N, and M218F, and optionally R203G;
(35) the amino acid substitutions Q74L/V/I/F/W/Y/C, S135G/F, I138T, R203G/Q and M218F/I/W/L, and optionally S153N/C/I/V;
(36) the amino acid substitutions Q74L, S135G, I138T, R203G, and M218F, and optionally S153N;
(37) the amino acid substitutions Q74L/V/I/F/W/Y/C, I138T, S153N/C/I/V, R203G/Q and M218F/I/W/L, and optionally S135G/F;
(38) the amino acid substitutions Q74L, I138T, S153N, R203G, and M218F, and optionally S135G;
(39) the amino acid substitutions Q74L/V/I/F/W/Y/C, S135G/F, I138T, S153N/C/I/V, R203G/Q and M218F/I/W/L; or
(40) the amino acid substitutions Q74L, S135G, I138T, S153N, R203G and M218F.

In various preferred embodiments, the polypeptide comprises
(1) the amino acid substitutions S135G/F, S153N/C/I/V and R203G/Q;
(2) the amino acid substitutions S135G, S153N and R203G;
(3) the amino acid substitutions Q74L/V/I/F/W/Y/C, S153N/C/I/V and R203G/Q;
(4) the amino acid substitutions Q74L, S153N and R203G;
(5) the amino acid substitutions I138T, S153N/C/I/V and R203G/Q;
(6) the amino acid substitutions I138T, S153N and R203G;
(7) the amino acid substitutions Q74L/V/I/F/W/Y/C, S135G/F, S153N/C/I/V and R203G/Q;
(8) the amino acid substitutions Q74L, S135G, S153N and R203G;
(9) the amino acid substitutions S135G/F, I138T, S153N/C/I/V and R203G/Q;
(10) the amino acid substitutions S135G, I138T, S153N and R203G;
(11) the amino acid substitutions Q74L/V/I/F/W/Y/C, S135G/F, I138T and R203G/Q;
(12) the amino acid substitutions Q74L, S135G, I138T and R203G;
(13) the amino acid substitutions Q74L/V/I/F/W/Y/C, S135G/F, I138T and S153N/C/IN;
(14) the amino acid substitutions Q74L, S135G, I138T and S153N;
(15) the amino acid substitutions Q74L/V/I/F/W/Y/C, I138T, S153N/C/IN and R203G/Q;
(16) the amino acid substitutions Q74L, I138T, S153N and R203G;
(17) the amino acid substitutions Q74L/V/I/F/W/Y/C, S135G/F, I138T, S153N/C/I/V and R203G/Q;
(18) the amino acid substitutions Q74L, S135G, I138T, S153N and R203G;
(19) the amino acid substitutions Q74L/V/I/F/W/Y/C, S135G/F, I138T, S153N/C/I/V, R203G/Q and M218F/I/W/L; or
(20) the amino acid substitutions Q74L, S135G, I138T, S153N, R203G and M218F.

In various embodiments, the polypeptide comprises only the specific mutations/set of mutations indicated herein with the remainder of the sequence being identical to the sequence set forth in SEQ ID NO:1.

In various embodiments, the polypeptide comprises the amino acid sequence set forth in any one of SEQ ID Nos. 4-17 and 24-30. In further embodiments, also encompassed are variants of these sequences that have at least 80%, at least 85%, at least 90%, at least 95%, at least 97.5%, at least 99% or at least 99.5% sequence identity with the amino acid sequence set forth in the respective template sequence of any one of SEQ ID Nos. 4-17 and 24-30. These variants include truncated versions of the sequences set forth in SEQ ID Nos. 4-17 and 24-30, for example N- or C-terminal truncations, with these truncations (i.e. the deleted amino acid stretches) typically being 1-10, preferably 1-5 amino acids in length. In such variants all the substitutions and invariable positions of the template sequence are retained.

In various embodiments, the amino acid sequence comprises as the first, N-terminal amino acid the residue M. If this is not present within the specific sequences disclosed herein, it may be artificially added, if desired, in particular to facilitate expression in a host cell. In various embodiments, the polypeptides disclosed herein may comprise an N-terminal extension comprising a 6×His-tag, such as the amino acid sequence set forth in SEQ ID NO:21.

In various embodiments, the present invention relates to a(n) (isolated) polypeptide comprising an amino acid sequence that has at least 80%, at least 85%, at least 90%, at least 95%, at least 97.5%, at least 99% or at least 99.5% sequence identity over its entire length with the amino acid sequence set forth in SEQ ID NO:1 and comprises one or more amino acid substitution(s) selected from the group consisting of: M218F/I/W/L, T22V/I/S/Y, T30A/S, D148N/R/Y, Q74L/V/I/F/W/Y/C, I138T and S135G/F, wherein the positions 46, 81 and 151 and, optionally, 17, 68, 77, and 219 are invariable, wherein the positional numbering is according to SEQ ID NO:1, or a functional fragment thereof, preferably having protease activity. In various embodiments, the positions 68, 77, (scaffold), 46, 81 and 151 (catalytic triad) are invariable, and the position 17 is S or A and the position 219 is N/V/D/E/P or K (scaffold), wherein the positional numbering is according to SEQ ID NO:1. In various embodiments, the polypeptide can additionally comprise any one or both of the substitutions: S153N/C/I/V and R203G/Q. In various embodiments, it is preferred that the polypeptides of the invention further comprise at least the R203G substitution. In various embodiments, the polypeptide of the invention may further comprise the R203G and the S153N substitution. In such embodiments, the R203G and/or the S153N substitution(s) may then also be invariable.

In various embodiments, the polypeptide comprises any one, two, three, four, five or six amino acid substitution of M218F/I/W/L, T22V/I/S/Y, T30A/S, D148N/R/Y, S153N/C/I/V and R203G/Q, and optionally one or more of Q74L/I/F/W/Y/C, S135G/F and I138T, wherein the positional numbering is according to SEQ ID NO:1.

Preferably, the polypeptide comprises the R203G/Q substitution, preferably the R203G substitution, and
(1) any one, two, or three amino acid substitutions of M218F/I/W/L, T22V/I/S/Y and T30A/S, preferably any one, two or three of M218F, T22I and T30A/S, and optionally one or more of Q74L/V/I/F/W/Y/C, S135G/F, I138T and S153N/C/I/V; or
(2) any one, two, three or four amino acid substitutions of M218F/I/W/L, T22V/I/S/Y, T30A/S and D148N/R/Y, preferably M218F, T22V, T30A and D148N, and optionally one or more of Q74L/V/I/F/W/Y/C, S135G/F, I138T and S153N/C/I/V;
wherein the positional numbering is according to SEQ ID NO:1.

In various embodiments, the polypeptide comprises the mutations T17S, N68D, I77V, R203G and S219N, and a deletion of the last five C-terminal amino acid residues (Δ238-242) relative to the TEV protease wildtype sequence (SEQ ID NO:2) (TEV protease variant SM: SEQ ID NO:31), and additionally the amino acid substitutions
(1) T22V, T30A, D148N and M218F (SEQ ID NO: 4);
(2) T22I, T30A, and M218F (SEQ ID NO: 6);
(3) T22S, T30A, D148N and M218F (SEQ ID NO: 5);
(4) T22I, T30S and M218F (SEQ ID NO: 11);
(5) T22I, T30A, D148R and M218F (SEQ ID NO: 7);
(6) T22Y, T30S, D148N and M218F (SEQ ID NO: 8);
(7) T22I, T30S, D148R and M218F (SEQ ID NO: 12);
(8) T22I, T30S, D148Y and M218F (SEQ ID NO: 9); or
(9) T22Y, T30S, D148R and M218F (SEQ ID NO: 10).

In various embodiments, the polypeptide comprises or consists of the amino acid sequences as set forth in any one of SEQ ID Nos. 4, 5, 6, 7, 8, 9, 10, 11 and 12, preferably SEQ ID NO:4.

In a further aspect, the polypeptide according to the invention comprises an amino acid sequence that has at least 80%, at least 85%, at least 90%, at least 95%, at least 97.5%, at least 99% or at least 99.5% sequence identity over its entire length with the amino acid sequence set forth in SEQ ID NO:1 and comprises the amino acid substitution Q74L/V/I/F/W/Y/C and optionally any one or more of S135G/F, I138T, S153N/C/I/V, R203G/Q and M218F/I/W/L; wherein the positions 68, 77, (scaffold), 46, 81 and 151 (catalytic triad) are invariable, and the position 17 is S or A and the position 219 is N/V/D/E/P or K (scaffold), wherein the positional numbering is according to SEQ ID NO:1, or a functional fragment thereof, wherein the polypeptide or the functional fragment has protease activity.

In various embodiments, the polypeptide may further comprise any one, two or three of the amino acid substitutions T22V/I/S/Y, T30A/S, D148N/R/Y.

Preferably, the amino acid sequence that has at least 80%, at least 85%, at least 90%, at least 95%, at least 97.5%, at least 99% or at least 99.5% sequence identity over its entire length with the amino acid sequence set forth in SEQ ID NO:1 at its C-terminus retains the deletion of amino acids 237-242 relative to the TEV protease wildtype sequence set forth in SEQ ID NO:2.

In various embodiments, the functional fragment is at least 202 amino acids in length and comprises amino acid residues corresponding to those at positions 17 to 218 of SEQ ID NO:1.

In various embodiments, the polypeptide comprises
(1) the amino acid substitutions Q74L/V/I/F/W/Y/C and S135G/F, and optionally any one or more of I138T, S153N/C/I/V, R203G/Q and M218F/I/W/L;
(2) the amino acid substitutions Q74L, and S135G, and optionally any one or more of I138T, S153N, R203G and M218F;
(3) the amino acid substitutions Q74L/V/I/F/W/Y/C and I138T, and optionally any one or more of S135G/F, S153N/C/I/V, R203G/Q and M218F/I/W/L;
(4) the amino acid substitutions Q74L and I138T, and optionally any one or more of S135G, S153N, R203G and M218F;
(5) the amino acid substitutions Q74LN/I/F/W/Y/C and S153N/C/IN, and optionally any one or more of S135G/F, I138T, R203G/Q and M218F/I/W/L;
(6) the amino acid substitutions Q74L, and S153N, and optionally any one or more of S135G, I138T, R203G and M218F;
(7) the amino acid substitutions Q74L/V/I/F/W/Y/C and R203G/Q, and optionally any one or more of S135G/F, I138T, S153N/C/I/V and M218F/I/W/L;
(8) the amino acid substitutions Q74L, and R203G, and optionally any one or more of S135G, I138T, S153N and M218F;
(9) the amino acid substitutions Q74L/V/I/F/W/Y/C and M218F/I/W/L, and optionally any one or more of S135G/F, I138T, S153N/C/I/V and R203G/Q;
(10) the amino acid substitutions Q74L, and M218F, and optionally any one or more of S135G, I138T, S153N and R203G;
(11) the amino acid substitutions Q74L/V/I/F/W/Y/C, S135G/F and I138T, and optionally any one, any two or three of S153N/C/I/V, R203G/Q and M218F/I/W/L;
(12) the amino acid substitutions Q74L, S135G and I138T, and optionally any one, any two or three of S153N, R203G and M218F;
(13) the amino acid substitutions Q74L/V/I/F/W/Y/C, S135G/F and S153N/C/I/V, and optionally any one, any two or three of I138T, R203G/Q and M218F/I/W/L;
(14) the amino acid substitutions Q74L, S135G, and S153N, and optionally any one, any two or three of I138T, R203G and M218F;
(15) the amino acid substitutions Q74L/V/I/F/W/Y/C, S135G/F and R203G/Q, and optionally any one, any two or three of I138T, S153N/C/I/V and M218F/I/W/L;
(16) the amino acid substitutions Q74L, S135G, and R203G, and optionally any one, any two or three of I138T, S153N and M218F;
(17) the amino acid substitutions Q74L/V/I/F/W/Y/C, S135G/F and M218F/I/W/L, and optionally any one, any two or three of I138T, S153N/C/I/V and R203G/Q;
(18) the amino acid substitutions Q74L, S135G, and M218F, and optionally any one, any two or three of I138T, S153N and R203G;
(19) the amino acid substitutions Q74L/V/I/F/W/Y/C, I138T and S153N/C/I/V, and optionally any one, any two or three of S135G/F, R203G/Q and M218F/I/W/L;
(20) the amino acid substitutions Q74L, I138T and S153N, and optionally any one, any two or three of S135G, R203G and M218F;
(21) the amino acid substitutions Q74L/V/I/F/W/Y/C, I138T and R203G/Q, and optionally any one, any two or three of S135G/F, S153N/C/I/V and M218F/I/W/L;
(22) the amino acid substitutions Q74L, I138T and R203G, and optionally any one, any two or three of S135G, S153N and M218F;
(23) the amino acid substitutions Q74L/V/I/F/W/Y/C, I138T and M218F/I/W/L, and optionally any one, any two or three of S135G/F, S153N/C/I/V and R203G/Q;
(24) the amino acid substitutions Q74L, I138T and M218F, and optionally any one, any two or three of S135G, S153N and R203G;
(25) the amino acid substitutions Q74L/V/I/F/W/Y/C, S153N/C/I/V and R203G/Q, and optionally any one, any two or three of S135G/F, I138T, M218F/I/W/L;
(26) the amino acid substitutions Q74L, S153N, and R203G, and optionally any one, any two or three of S135G, I138T and M218F;
(27) the amino acid substitutions Q74L/V/I/F/W/Y/C, S153N/C/I/V and M218F/I/W/L, optionally any one, any two or three of S135G/F, I138T, R203G/Q;
(28) the amino acid substitutions Q74L, S153N, and M218F, and optionally any one, any two or three of S135G, I138T and R203G;
(29) the amino acid substitutions Q74L/V/I/F/W/Y/C, R203G/Q and M218F/I/W/L, and optionally any one, any two or three of S135G/F, I138T, S153N/C/I/V;
(30) the amino acid substitutions Q74L, R203G, and M218F, and optionally any one, any two or three of S135G, I138T and S153N;
(31) the amino acid substitutions Q74L/V/I/F/W/Y/C, S135G/F, I138T and S153N/C/IN, and optionally any one or two of R203G/Q and M218F/I/W/L;
(32) the amino acid substitutions Q74L, S135G, I138T and S153N, and optionally any one or two of R203G and M218F;
(33) the amino acid substitutions Q74L/V/I/F/W/Y/C, S135G/F, I138T and R203G/Q, and optionally any one or two of S153N/C and M218F/I/W/L;
(34) the amino acid substitutions Q74L, S135G, I138T and R203G, and optionally any one or two of S153N and M218F;
(35) the amino acid substitutions Q74L/V/I/F/W/Y/C, S135G/F, I138T and M218F/I/W/L, and optionally any one or two of S153N/C/IN and R203G/Q;
(36) the amino acid substitutions Q74L, S135G, I138T and M218F, and optionally any one or two of S153N and R203G;
(37) the amino acid substitutions Q74L/V/I/F/W/Y/C, S135G/F, S153N/C/I/V, and R203G/Q, and optionally any one or two of I138T and M218F/I/W/L;
(38) the amino acid substitutions Q74L, S135G, S153N and R203G, and optionally any one or two of I138T and M218F;
(39) the amino acid substitutions Q74L/V/I/F/W/Y/C, S135G/F, S153N/C/I/V and M218F/I/W/L, and optionally any one or two of I138T and R203G/Q;
(40) the amino acid substitutions Q74L, S135G, S153N and M218F, and optionally any one or two of I138T and R203G;
(41) the amino acid substitutions Q74L/V/I/F/W/Y/C, I138T, S153N/C/I/V and R203G/Q, and optionally any one or two of S135G/F and M218F/I/W/L;
(42) the amino acid substitutions Q74L, I138T, S153N, and R203G, and optionally any one or two of S135G and M218F;
(43) the amino acid substitutions Q74L/V/I/F/W/Y/C, I138T, S153N/C/I/V and M218F/I/W/L, and optionally any one or two of S135G/F and R203G/Q;
(44) the amino acid substitutions Q74L, I138T, S153N, and M218F, and optionally any one or two of S135G and R203G;
(45) the amino acid substitutions Q74L/V/I/F/W/Y/C, S153N/C, R203G/Q and M218F/I/W/L, and optionally any one or two of S135G/F and I138T;
(46) the amino acid substitutions Q74L, S153N, R203G, and M218F, and optionally any one or two of S135G and I138T;
(47) the amino acid substitutions Q74L/V/I/F/W/Y/C, S135G/F, R203G/Q, and M218F/I/W/L, and optionally any one or two of I138T and S153N/C/I/V;
(48) the amino acid substitutions Q74L, S135G, R203G, and M218F, and optionally any one or two of I138T and S153N;
(49) the amino acid substitutions Q74L/V/I/F/W/Y/C, S135G/F, I138T, S153N/C/I/V and R203G/Q, and optionally M218F/I/W/L;
(50) the amino acid substitutions Q74L, S135G, I138T, S153N, and R203G, and optionally M218F;
(51) the amino acid substitutions Q74L/V/I/F/W/Y/C, S135G/F, I138T, S153N/C/I/V and M218F/I/W/L, and optionally R203G/Q;
(52) the amino acid substitutions Q74L, S135G, I138T, S153N, and M218F, and optionally R203G;
(53) the amino acid substitutions Q74L/V/I/F/W/Y/C, S135G/F, I138T, R203G/Q and M218F/I/W/L, and optionally S153N/C/I/V;
(54) the amino acid substitutions Q74L, S135G, I138T, R203G, and M218F, and optionally S153N;
(55) the amino acid substitutions Q74L/V/I/F/W/Y/C, S135G/F, S153N/C/I/V, R203G/Q and M218F/I/W/L, and optionally I138T;
(56) the amino acid substitutions Q74L, S135G, S153N, R203G, and M218F, and optionally I138T;
(57) the amino acid substitutions Q74L/I/F/W/Y/C, I138T, S153N/C/I/V, R203G/Q and M218F/I/W/L, and optionally S135G/F;
(58) the amino acid substitutions Q74L, I138T, S153N, R203G and M218F, and optionally S135G;
(59) the amino acid substitutions Q74L/V/I/F/W/Y/C, S135G/F, I138T, S153N/C/I/V, R203G/Q and M218F/I/W/L; or
(60) the amino acid substitutions Q74L, S135G, I138T, S153N, R203G and M218F.

In various embodiments, the polypeptide may further comprise any one, two or three of the amino acid substitutions T22V/I/S/Y, T30A/S, D148N/R/Y.

In various embodiments, the polypeptide comprises
(1) the amino acid substitutions Q74L/V/I/F/W/Y/C, S153N/C/I/V and R203G/Q;
(2) the amino acid substitutions Q74L, S153N and R203G;
(3) the amino acid substitutions Q74L/V/I/F/W/Y/C, S135G/F, S153N/C/I/V and R203G/Q;
(4) the amino acid substitutions Q74L, S135G, S153N and R203G;
(5) the amino acid substitutions Q74L/V/I/F/W/Y/C, I138T, S153N/C/IN and R203G/Q;
(6) the amino acid substitutions Q74L, I138T, S153N and R203G;
(7) the amino acid substitutions Q74L/V/I/F/W/Y/C, S135G/F, I138T, S153N/C/IN and R203G/Q;
(8) the amino acid substitutions Q74L, S135G, I138T, S153N and R203G;
(9) the amino acid substitutions Q74L/V/I/F/W/Y/C, S135G/F, I138T and R203G/Q;
(10) the amino acid substitutions Q74L, S135G, I138T and R203G;
(11) the amino acid substitutions Q74L/V/I/F/W/Y/C, S135G/F, I138T, S153N/C/I/V, R203G/Q and M218F/I/W/L; or
(12) the amino acid substitutions Q74L, S135G, I138T, S153N, R203G and M218F;
wherein the positional numbering is according to SEQ ID NO:1.

In various embodiments, the polypeptides of the invention may further comprise any one or more of the amino acid substitution(s) L60W, F5C and L210C, wherein the positional numbering is according to SEQ ID NO:1.

Preferably, the polypeptide is an isolated polypeptide.

In various embodiments,
(a) the polypeptide according to the invention is up to 236 amino acids in length; and/or
(b) the polypeptide according to the invention comprises or consists of the amino acid sequence set forth in any one of SEQ ID Nos. 13 to 17, 24, 27, 28 or 30.

The invention further relates to the nucleic acid, in particular the isolated nucleic acid molecule, encoding the polypeptide(s) as described above. The polypeptide may comprise in addition to the amino acid sequence defined herein other amino acid sequences that encode different (poly)peptides, for example (poly)peptide tags that are used or useful for detection or purification. Known tags include, without limitation, the 6×His tag. These may be linked to the amino acid sequence defined herein by a linker peptide sequence, which is typically 1 to 20 amino acids in length, for example 2 to 10 amino acids, and may be a glycine-rich sequence. Such peptide tags are preferably attached to the N-terminal end of the amino acid sequence defined herein. These amino acid sequences are typically linked by peptide bonds and expressed as a single fusion protein.

In certain embodiments, the above defined nucleic acid molecules may be comprised in a vector, for example a cloning or expression vector. Generally, the nucleic acid molecules of the invention can also be part of a vector or any other kind of cloning vehicle, including, but not limited to a plasmid, a phagemid, a phage, a baculovirus, a cosmid, or an artificial chromosome. Generally, a nucleic acid molecule disclosed in this application may be “operably linked” to a regulatory sequence (or regulatory sequences) to allow expression of this nucleic acid molecule.

Such cloning vehicles can include, besides the regulatory sequences described above and a nucleic acid sequence of the present invention, replication and control sequences derived from a species compatible with the host cell that is used for expression as well as selection markers conferring a selectable phenotype on transformed or transfected cells. Large numbers of suitable cloning vectors are known in the art, and are commercially available.

In certain embodiments the nucleic acid molecules disclosed herein are comprised in a cloning vector. In some embodiments the nucleic acid molecules disclosed herein are comprised in an expression vector. The vectors may comprise regulatory elements for replication and selection markers. In certain embodiments, the selection marker may be selected from the group consisting of genes conferring ampicillin, kanamycin, chloramphenicol, tetracycline, blasticidin, spectinomycin, gentamicin, hygromycin, and zeocin resistance. In various other embodiments, the selection may be carried out using antibiotic-free systems, for example by using toxin/antitoxin systems, cer sequence, triclosan, auxotrophies or the like. Suitable methods are known to those skilled in the art.

The above-described nucleic acid molecule of the present invention, if integrated in a vector, must be integrated such that the polypeptide can be expressed. Therefore, a vector of the present invention comprises sequence elements which contain information regarding to transcriptional and/or translational regulation, and such sequences are “operably linked” to the nucleotide sequence encoding the polypeptide. An operable linkage in this context is a linkage in which the regulatory sequence elements and the sequence to be expressed are connected in a way that enables gene expression. The precise nature of the regulatory regions necessary for gene expression may vary among species, but in general these regions comprise a promoter which, in prokaryotes, contains both the promoter per se, i.e. DNA elements directing the initiation of transcription, as well as DNA elements which, when transcribed into RNA, will signal the initiation of translation. Such promoter regions normally include 5′ non-coding sequences involved in initiation of transcription and translation, such as the −35/−10 boxes and the Shine-Dalgarno element in prokaryotes or the TATA box, CAAT sequences, and 5′-capping elements in eukaryotes. These regions can also include enhancer or repressor elements as well as translated signal and leader sequences for targeting the native polypeptide to a specific compartment of a host cell.

In addition, the 3′ non-coding sequences may contain regulatory elements involved in transcriptional termination, polyadenylation or the like. If, however, these termination sequences are not satisfactory functional in a particular host cell, then they may be substituted with signals functional in that cell.

In various embodiments, a vector comprising a nucleic acid molecule of the invention can therefore comprise a regulatory sequence, preferably a promoter sequence. In certain embodiments, the promoter is identical or homologous to promoter sequences of the host genome. In such cases endogenous polymerases may be capable to transcribe the nucleic acid molecule sequence comprised in the vector. In various embodiments, the promoter is selected from the group of weak, intermediate and strong promoters, preferably from weak to intermediate promoters.

In another preferred embodiment, a vector comprising a nucleic acid molecule of the present invention comprises a promoter sequence and a transcriptional termination sequence. Suitable promoters for prokaryotic expression are, for example, the araBAD promoter, the tet-promoter, the lacUV5 promoter, the CMV promo tor, the EF1 alpha promotor, the AOX1 promotor, the tac promotor, the T7 promoter, or the lac promotor. Examples of promoters useful for expression in eukaryotic cells are the SV40 promoter or the CMV promoter. Furthermore, a nucleic acid molecule of the invention can comprise transcriptional regulatory elements, e.g., repressor elements, which allow regulated transcription and translation of coding sequences comprised in the nucleic acid molecule. Repressor element may be selected from the group consisting of the Lac-, AraC-, or MalR-repressor.

The vector may be effective for prokaryotic or eukaryotic protein expression. In particular, the nucleic acid molecules of the present invention may be comprised in a vector for prokaryotic protein expression. Such vector sequences are constructed such that a sequence of interest can easily be inserted using techniques well known to those skilled in the art. In certain embodiments, the vector is selected from the group consisting of a pET-vector, a pBAD-vector, a pK184-vector, a pMONO-vector, a pSELECT-vector, pSELECT-Tag-vector, a pVITRO-vector, a pVIVO-vector, a pORF-vector, a pBLAST-vector, a pUO-vector, a pDUO-vector, a pZERO-vector, a pDeNy-vector, a pDRIVE-vector, a pDRIVE-SEAP-vector, a HaloTag®Fusion-vector, a pTARGET™-vector, a Flexi®-vector, a pDEST-vector, a pHIL-vector, a pPIC-vector, a pMET-vector, a pPink-vector, a pLP-vector, a pTOPO-vector, a pBud-vector, a pCEP-vector, a pCMV-vector, a pDisplay-vector, a pEF-vector, a pFL-vector, a pFRT-vector, a pFastBac-vector, a pGAPZ-vector, a pIZ/V5-vector, a p3S-vector, a plAR-vector, pSEC, pMS, a pSU2726-vector, a pLenti6-vector, a pMIB-vector, a pOG-vector, a pOpti-vector, a pREP4-vector, a pRSET-vector, a p SCREEN-vector, a pSecTag-vector, a pTEFI-vector, a pTracer-vector, a pTrc-vector, a pUB6-vector, a pVAXI-vector, a pYC2-vector, a pYES2-vector, a pZeo-vector, a pcDNA-vector, a pFLAG-vector, a pTAC-vector, a pT7-vector, a Gateway®-vector, a pQE-vector, a pLEXY-vector, a pRNA-vector, a pPK-vector, a pUMVC-vector, a pLIVE-vector, a pCRUZ-vector, a Duet-vector, and other vectors or derivatives thereof.

The vectors of the present invention may be chosen from the group consisting of high, medium and low copy vectors.

The above described vectors of the present invention may be used for the transformation or transfection of a host cell in order to achieve expression of a polypeptide which is encoded by an above described nucleic acid molecule and comprised in the vector DNA. Thus, in a further aspect, the present invention also relates to a host cell comprising a vector or nucleic acid molecule as disclosed herein.

Also contemplated herein are host cells, which comprise a nucleic acid molecule as described herein integrated into their genomes. The skilled person is aware of suitable methods for achieving the nucleic acid molecule integration. For example, the molecule may be delivered into the host cells by means of liposome transfer or viral infection and afterwards the nucleic acid molecule may be integrated into the host genome by means of homologous recombination. In certain embodiments, the nucleic acid molecule is integrated at a site in the host genome, which mediates transcription of the peptide or protein of the invention encoded by the nucleic acid molecule. In various embodiments, the nucleic acid molecule further comprises elements which mediate transcription of the nucleic acid molecule once the molecule is integrated into the host genome and/or which serve as selection markers.

In certain embodiments, the nucleic acid molecule of the present invention is transcribed by a polymerase natively encoded in the host genome. In various embodiments, the nucleic acid molecule is transcribed by an RNA-polymerase which is non-native to the host genome. In such embodiments, the nucleic acid molecule of the present invention may further comprise a sequence encoding for a polymerase and/or the host genome may be engineered or the host cell may be infected to comprise a nucleic acid sequence encoding for an exogenous polymerase. The host cell may be specifically chosen as a host cell capable of expressing the gene. In addition or otherwise, in order to produce the polypeptide of the invention, the nucleic acid coding for it can be genetically engineered for expression in a suitable system. Transformation can be performed using standard techniques (Sambrook, J. et al. (2001), Molecular Cloning: A Laboratory Manual, 3rd Ed., Cold Spring Harbor Laboratory Press, Cold Spring Harbor, NY).

Prokaryotic or eukaryotic host organisms comprising such a vector for recombinant expression of the polypeptide as described herein form also part of the present invention. Suitable host cells can be prokaryotic cells. In certain embodiments the host cells are selected from the group consisting of gram-positive and gram-negative bacteria. In some embodiments, the host cell is a gram-negative bacterium, such as E. coli. In certain embodiments, the host cell is E. coli, in particular E. coli BL21 (DE3) or other E. coli K12 or E. coli B834 derivatives. In further embodiments, the host cell is selected from the group consisting of Escherichia coli (E. coli), Pseudomonas, Serratia marcescens, Salmonella, Shigella (and other enterobacteriaceae), Neisseria, Hemophilus, Klebsiella, Proteus, Enterobacter, Helicobacter, Acinetobacter, Moraxella, Helicobacter, Stenotrophomonas, Bdellovibrio, Legionella, acetic acid bacteria, Bacillus, Bacilli, Carynebacterium, Clostridium, Listeria, Streptococcus, Staphylococcus, and Archaea cells. Suitable eukaryotic host cells are among others CHO cells, insect cells, fungi, yeast cells, e.g., Saccharomyces cerevisiae, S. pombe, Pichia pastoris.

In certain embodiments, the host cell is a prokaryotic cell, such as E. coli, in particular E. coli BL21 (DE3), E. coli BL21, E. coli K12, E. coli BLR, E. coli BL21 AI, E. coli BL21 μLysS, E. coli XL1 and E. coli DH5a. Further suitable E. coli strains include, but are not limited to DH1, DH5a, DM1, HB101, JMIOI-110, Rosetta(DE3)pLysS, SURE, TOP10, XLI-Blue, XL2-Blue and XLIO-Blue strains.

The transformed host cells are cultured under conditions suitable for expression of the nucleotide sequence encoding the polypeptide of the invention. In certain embodiments, the cells are cultured under conditions suitable for expression of the nucleotide sequence encoding a polypeptide of the invention and, optionally, its secretion.

For producing the recombinant polypeptide described herein, a vector of the invention can be introduced into a suitable prokaryotic or eukaryotic host organism by means of recombinant DNA technology (as already outlined above). For this purpose, the host cell is first transformed with a vector comprising a nucleic acid molecule according to the present invention using established standard methods (Sambrook, J. et al. (2001), supra). The host cell is then cultured under conditions, which allow expression of the heterologous DNA and thus the synthesis of the corresponding polypeptide. Subsequently, the polypeptide is recovered either from the cell or from the cultivation medium.

For expression of the polypeptides of the present invention several suitable protocols are known to the skilled person. The expression of a recombinant polypeptide of the present invention may be achieved by the following method comprising: (a) introducing a nucleic acid molecule or vector of the invention into a host cell, wherein the nucleic acid molecule or vector encodes the recombinant polypeptide; and (b) cultivating the host cell in a culture medium under conditions that allow expression of the recombinant polypeptide.

Step (a) may be carried out by using suitable transformation and transfection techniques known to those skilled in the art. These techniques are usually selected based on the type of host cell into which the nucleic acid is to be introduced. In some embodiments, the transformation may be achieved using electroporation or heat shock treatment of the host cell.

Step (b) may include a cultivation step that allows growth of the host cells. Alternatively, such step allowing growth of the host cells and a step that allows expression of the polypeptide may be performed separately in that the cells are first cultivated such that they grow to a desired density and then they are cultivated under conditions that allow expression of the polypeptide. The expression step can however still allow growth of the cells.

The method may further include a step of recovering the expressed polypeptide. The polypeptide may be recovered from the growth medium, if it is secreted, or from the cells or both. The recovery of the polypeptide may include various purification steps.

Generally, any known culture medium suitable for growth of the selected host may be employed in this method. In various embodiments, the medium is a rich medium or a minimal medium. Also contemplated herein is a method, wherein the steps of growing the cells and expressing the peptide or protein comprise the use of different media. For example, the growth step may be performed using a rich medium which is replaced by a minimal medium in the expression step. In certain cases, the medium is selected from the group consisting of LB medium, TB medium, 2YT medium, synthetical medium and minimal medium.

In various embodiments, the method also encompasses the purification of the recombinant polypeptide, wherein the recombinant polypeptide is purified using a method selected from affinity chromatography, ion exchange chromatography, reverse phase chromatography, size exclusion chromatography, and combinations thereof.

In a further aspect, the present invention relates to the use of a vector or nucleic acid molecule as disclosed herein for the expression of a recombinant polypeptide. In some embodiments, the vector is used for the expression and optionally secretion of a recombinant polypeptide. The expression or expression and secretion may be achieved using the method described herein.

A method for expression of a recombinant polypeptide using the above-described nucleic acid molecules may comprise the steps of:
(a) introducing a nucleic acid molecule or a vector as described above into a suitable host cell, wherein the nucleic acid molecule or vector encodes the recombinant polypeptide; and
(b) cultivating the host cell in a culture medium under conditions that allow expression of the recombinant polypeptide.

The invention further relates to methods for the proteolytic cleavage of a substrate peptide or polypeptide. These substrate polypeptides typically comprise the specific TEV protease recognition and cleavage site with the amino acid sequence set forth in SEQ ID NO:18 (ENLYFQX), wherein X can be any amino acid, preferably G, S, V, I, L, K, R, T, or E, for example G or V. While the wildtype TEV protease is highly specific for G as residue X, at the so-called P1′ position, it is preferred that the variants allow other amino acids at this position without affecting catalytic activity and efficiency. As the C-terminal amino acid at position X of the motif remains in the cleaved C-terminal part, and the TEV cleavage site if often located on the N-terminus of a polypeptide of interest, it is advantageous if the TEV variant can accommodate different amino acids in this position to avoid an undesired substitution in this position. The variants of TEV protease disclosed herein have been found to have an altered substrate specificity with respect to this position and thus represent an improvement over TEV wildtype.

In the methods for the cleavage of a substrate polypeptide, the TEV protease variant of the invention is contacted with the substrate polypeptide under conditions that allow the cleavage of the polypeptide. Such conditions are generally known to those skilled in the art and may include specific buffer conditions and temperature control. After the cleavage reaction has occurred, the cleaved polypeptide may be subjected to further purification steps in order to separate it from the other fragment and/or the protease. Suitable purification protocols are known to those skilled in the art and include chromatographic methods, such as gel filtration, ion exchange or affinity chromatography, without being limited thereto.

In a further aspect, the present invention is also directed to the use of the polypeptides disclosed herein for cleavage of a substrate polypeptide as defined herein above.

In all methods and uses disclosed herein, the substrate polypeptide may be a fusion protein, preferably a non-natural fusion protein. Such fusion proteins typically comprise at least two different protein parts, domains or regions that may be connected by a linker that includes the TEV protease site. After expression and isolation, the different parts, domains or regions may thus be separated from each other by subjecting the fusion protein to a cleavage reaction with TEV protease variants and subsequent purification/separation.

The present invention further encompasses kits and compositions that comprise the polypeptides, nucleic acids, vectors and/or host cells of the invention. Such kits may further comprise instructions for use and/or buffers and other materials to allow directed use and application of the polypeptides.

### EXAMPLES

### Materials and Methods

### Pre-Culture

Individual clones were picked and inoculated in sterile 96-well plates, referred to as master plates, containing 100 μL of LB media, supplemented with 34 μg/mL Chloramphenicol+30 μg/mL Kanamycin, per well. Plates were sealed with parafilm and incubated at 37° C., 250 rpm and 80% relative humidity in a humidity shaker overnight.

### Main Culture

Next day plates were replicated with cryo replicator into new 96-deep well plates containing 300 μL of TB media supplemented with 34 μg/mL Chloramphenicol, 30 μg/mL Kanamycin and 2 mM MgCl₂. Plates were incubated at 37° C., 250 rpm and 80% relative humidity approximately 5 hours. When OD600 reached 0.6-0.8, in each well 100 μL of TB media supplemented with 34 μg/mL Cm+30 μg/mL Km+2 mM MgCl₂ and IPTG in a final concentration of 0.1 mM were added. Plates were further incubated for 19 h, 250 rpm, 80% relative humidity at 25° C.

### Cell Disruption

Plates were centrifuged (Eppendorf 5810R centrifuge, Germany) for 30 min, 3500 rpm at 4° C. and supernatant was discarded. In each cell pellet 150 μL of Bug Buster solution was added using multidrop dispenser (Multidrop Combi Reagent Dispenser, Thermo Scientific, USA) and plates were shaken in a plate shaker until the pellet was resuspended (few minutes). After that, plates were incubated in a humidity shaker for 20 min, 25° C., 250 rpm. In the next step plates were centrifuged for 40 min, 3500 rpm at 4° C., obtaining lysate solution.

### Activity Assay

20 μL of the lysate was transferred from the 96-deep well plate using liquid handler robotic station (Freedom EVO, Tecan, Switzerland) to a microplate already containing 80 μL of high purity water (1/5 dilution). Afterwards, from the 1/5 dilution microplate, 50 μL were transferred by liquid handler robotic station to a new microplate containing 50 μL of assay buffer 2× (100 mM Tris-HCl, 1 mM EDTA) (1/10 dilution). Next, 25 μL of 1/10 dilution were transferred to empty opaque microplates, 25 μL of previously prepared substrate solution (2 μL substrate (diluted in DMSO, 1 mM), 2 μL DTT (1M) in assay buffer) was added and fluorescence was measured immediately in plate reader (SPECTRAMax Plus 384, Molecular Devices, USA). Excitation was performed at a wavelength of 340 nm and emission was recorded at 490 nm.

### Cloning of the Variants

P1534 plasmid (pSF593 plasmid vector with N-terminal 6×His-tag and TEV double mutant (SEQ ID NO:3) insert with N-terminal tag (SEQ ID NO:21)) was digested with restriction enzymes BamHI-HF and HindIII-HF (NEB, USA). Cloning was performed with Gibson Assembly® (NEB, USA) and reactions were transformed into Escherichia coli XL2-Blue Ultracompetent Cells (Agilent, USA). The circular plasmids were retrieved through miniprep and sequenced. Once the sequences were confirmed to be correct, they were transformed together with pRIL plasmid in E. coli BL21 cells following the protocol.

### Expression of Variants in Flasks

Growing and expression of variants in flask was performed in LB-media using a pre- and main culture cultivation. The pre-cultures were inoculated with one colony from plates in 5 mL LB media, supplemented with 30 μg/mL Kanamycin and 34 μg/mL chloramphenicol. Cells were grown over night at 30° C., 180 rpm.

Main cultures in TB-media were set in 250 mL Erlenmeyer baffled flasks in a total volume of 25 mL media, supplemented with 30 μg/mL Kanamycin, 34 μg/mL chloramphenicol and 2 mM MgCl₂. Main cultures were inoculated with pre-culture corresponding to an optical density (600 nm) of ˜0.1 from pre-culture and cells were incubated at 30° C., 180 rpm, until and cell density of 0.6-0.8 incubator was set to 25° C. and induced with 0.1 mM IPTG incubation was continued for 19 h, 120 rpm, at 25° C.

Activity was measured with TEV Protease Activity Assay Kit from Abcam (UK).

### Thermostability Evaluation

To evaluate stability in the obtained mutants, dilution 1/5 of Bugbuster lysate (5 μL of the lysate in 20 μL of MilliQ water) was heated 10 min at 45° C. Afterwards it was introduced in ice for 10 min and chilled at room temperature 5 min. 5 μL of dilution 1/5 were added to 95 μL of assay buffer (dilution 1/100) and 25 μL of that dilution were mixed with 25 μL of substrate solution to evaluate activity.

### Fluorogenic Peptides

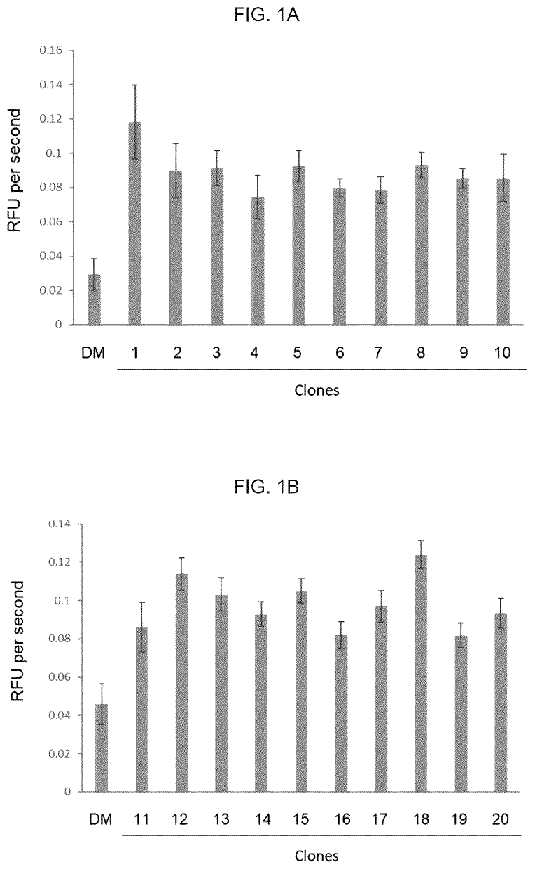

Peptides were designed attached to fluorophore EDANS (5-[(2-Aminoethyl)amino]naphthalene-1-sulfonic acid) and quencher Dabcyl (4-([4′-dimethylamino)phenyl]azo)benzoyl) to give a fluorometric response when cleaved. Each peptide was synthetized by Pepscan (The Netherlands) with {E(Edans)}=Edans attached to the N-terminal glutamic acid sidechain, {K(Dabcyl)}=Dabcyl attached to the C-terminal lysine sidechain of the substrate ENLYFQXGGK (X=G, V, I, L, K, R, T or E; SEQ ID NO:22). Excitation was performed at a wavelength of 340 nm and emission was recorded at 490 nm (FIGS. 1A-1B).

Activity Assay from Flask Expression

2 mL aliquot of cultivation broth were centrifuged (10 min, 15000 rpm) and the supernatant is discarded. The cell pellet is resuspended in 160 μL of Bug Buster solution and incubating (20 min, 25° C., 600 rpm) afterwards. Afterwards, centrifugation (20 min, 16000 rcf) is performed and 100 μL of the supernatant of the lysate is placed on ice. 5 μL of the lysate was diluted in 20 μL MQ H₂O (DF 1/5). 5 μL of this dilution is mixed in 45 μL of assay buffer 2′ (100 mM Tris-HCl, 1 mM EDTA) (DF 1/50). 25 μL of the lysate (DF 1/50) of each sample are pipetted into the 96 well plate as triplicate. Afterwards, 25 μL of the prepared substrate solution (2 μL substrate (diluted in DMSO, 1 mM), 2 μL DTT (1M) in assay buffer) are added swiftly to each well and measurement started immediately in plate reader (SPECTRAMax Plus 384, Molecular Devices, USA). Excitation was performed at a wavelength of 340 nm and emission was recorded at 490 nm.

### FuncLib Design

FuncLib is an automated method for designing multipoint mutations at enzyme active sites using phylogenetic analysis and Rosetta design calculations. In order to obtain the FuncLib variants, the positions selected were studied by the algorithm and from the pool of 50 variants, 26 were chosen for experiments after close inspection of each mutation.

### Microplate Expression of FuncLib Variants and Activity Assay

5 individual colonies of each variant were evaluated following the protocol developed at EvoEnzyme for microplate growing. P1 rev was used as positive control and for comparison purposes. Activity was evaluated with the 8 substrates, upscaling consequently the total volume used in dilution steps to have enough for all measurements.

TABLE 1
Overview of the variants generated and tested (substitutions relative to SEQ ID NO: 1)

| | SEQ | | | | | | | | |
|---|---|---|---|---|---|---|---|---|---|
| | ID NO: | | | | | | | | |
| DM | 3 | S153N | R203G | | | | | | |
| (P1534) | | | | | | | | | |
| 1 | 4 | T22V | T30A | D148N | R203G | M218F | | | |
| 2 | 6 | T22I | T30A | R203G | M218F | | | | |
| 3 | 4 | T22V | T30A | D148N | R203G | M218F | | | |
| 4 | 5 | T22S | T30A | D148N | R203G | M218F | | | |
| 5 | 11 | T22I | T30S | R203G | M218F | | | | |
| 6 | 5 | T22S | T30A | D148N | R203G | M218F | | | |
| 7 | 6 | T22I | T30A | R203G | M218F | | | | |
| 8 | 7 | T22I | T30A | D148R | R203G | M218F | | | |
| 9 | 5 | T22S | T30A | D148N | R203G | M218F | | | |
| 10 | 5 | T22S | T30A | D148N | R203G | M218F | | | |
| 11 | 8 | T22Y | T30S | D148N | R203G | M218F | | | |
| 12 | 5 | T22S | T30A | D148N | R203G | M218F | | | |
| 13 | 12 | T22I | T30S | D148R | R203G | M218F | | | |
| 14 | 12 | T22I | T30S | D148R | R203G | M218F | | | |
| 15 | 8 | T22Y | T30S | D148N | R203G | M218F | | | |
| 16 | 11 | T22I | T30S | R203G | M218F | | | | |
| 17 | 12 | T22I | T30S | D148R | R203G | M218F | | | |
| 18 | 5 | T22S | T30A | D148N | R203G | M218F | | | |
| 19 | 9 | T22I | T30S | D148Y | R203G | M218F | | | |
| 20 | 10 | T22Y | T30S | D148R | R203G | M218F | | | |
| P1 | 13 | Q74L | S135G | I138T | S153N | R203G | | | |
| P2687) | | | | | | | | | |
| P3 | 14 | Q74L | S135G | I138H | S153N | R203G | L60W | F5C | L210C |
| P4* | 15 | Q74L | S135G | I138H | S153N | R203G | L60W | F5C | L210C |
| P5* | 16 | Q74L | S135G | I138H | S153N | R203G | L60W | F5C | L210C |
| P1rev | 17 | Q74L | S135G | I138T | R203G | | | | |

1 = 3; 2 = 7; 4 = 6 = 9 = 10 = 12 = 18; 5 = 16; 11 = 15; 13 = 14 = 17
*P4 additionally comprised S200K, L76P, C130T and A206T
*P5 additionally comprised S200K, L76P, C130T, A206T, T118E, T113G, D78E, and D127E

### Example 1: Screening Assay

Standard TEV (SEQ ID NO:1), a known double mutant (DM; SEQ ID NO:3) and P8ref (negative control; SEQ ID NO:23) were grown in microplate format and TEV activity was assessed with Gly peptide (Edans-ENLYFQGGGK-Dabcyl); SEQ ID NO:19) using original activity assay protocols for flask expression and also using the modified activity assay protocol for 96-well expression described above. Fluorescence activity levels were significantly higher when modified protocol was employed (FIGS. 1A-1B). Also, activity was sufficiently high to serve as a control screening.

Applying the same modified protocol, screening assay was validated with standard TEV (SEQ ID NO:1) and Gly peptide (SEQ ID NO:19). 58 clones of standard TEV were assayed and an acceptable coefficient of variation (CV) of 15% was obtained (data not shown).

Prior to library screening standard TEV, DM and p8ref (negative control) were tested with the Val peptide (SEQ ID NO:20), the target for further screening, in order to estimate screening expectations. We observed consistent activity, albeit low, with the DM (SEQ ID NO:3), while with our parental type standard TEV (SEQ ID NO:1) no activity was detected.

A library of 320 individual mutants was transformed to the BL21 E. coli competent cells using pRIL as co-expression plasmid. Applying modified growing/assay protocol 896 individual clones, inoculated in 11 microplates, were screened with both, Gly and Val peptides, respectively. In each plate column 6 was inoculated with standard TEV (SEQ ID NO:1), while column 7 was inoculated with DM (SEQ ID NO:3), to allow reliable comparisons. In addition, in each plate a negative control was used.

Considering parental type did not show activity against Val peptide, clones in the library were compared to the DM (SEQ ID NO:3). 119 clones showed higher activity than the CV threshold (21%), while 78 clones displayed more than 2-fold improvement over DM (data not shown). After careful data analysis, 20 clones were selected for the rescreening process.

Selected clones were regrown, plasmids were extracted and BL21 E. coli was re-transformed. 20 clones, together with standard TEV and DM were inoculated in 2 microplates, having each clone picked 8 times in a single column (Plate 1—clones 1 to 10; Plate 2—clones 11 to 20). All clones reported herein were expressed with an N-terminal 6×His-tag and a G-rich linker sequence (SEQ ID NO:21). Again, in each plate column 6 was inoculated with standard TEV, while column 7 was inoculated with DM. Applying the same modified growing/assay protocol, mutants were screened with both peptides.

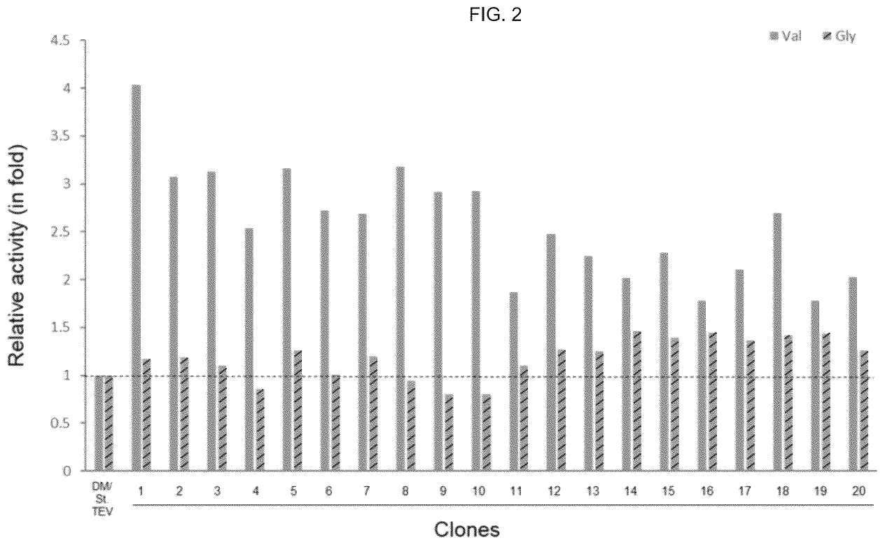

All clones showed significantly higher activity with the Val peptide than the DM confirming our screening results (FIGS. 1A-1B). Furthermore, plates were also screened with Gly peptide and all 20 clones retained their respective activities (FIG. 2).

Afterwards, all 20 clones were sent to sequencing and the results are depicted in Table 1. All clones additionally comprised the N-terminal sequence set forth in SEQ ID NO:21 to allow purification. As expected, there were repetition in the mutants due to the library nature. In total, 9 different clones were identified. Original parental type already contained mutation R203G. Mutation M218F was present in all clones and was found to be important for altered activity towards Val peptide. In position 148, we observed 3 changes (N, R and Y). However, it is the only position that was not changed in some of the clones and still good activity was observed. In position 30, there were two changes, A and S, where clones with A showed higher activity in general. Lastly, in position 22, we identified all 4 possible changes (V, I, S and Y).

### Example 2: Rational Design of TEV Variants

Positions selected to be fixed (maintain unaltered) in order to avoid activity loss were the following: 17, 46, 68, 77, 81, 151, 153, 203 and 219. The quality of the alignment was optimal, as there were a lot of sequences with a high coverage and also the sequence identity was low enough to have good diversity but not to be too far from TEV protease sequence. Different mutants were obtained with the regular PROSS algorithm and mutations close to important residues were deselected. Non redundant mutants were selected.

Mutants were cloned and expressed in flask for activity and thermostability studies. Four mutants P1, P3, P4 and P5 were found to surpassed DM's (SEQ ID NO:3) activity, the rest had similar values as standard TEVp (SEQ ID NO:1) and the mutant P8ref (SEQ ID NO: 23) was found to have no activity at all. It was verified that the specific activity of the variants has been increased by ruling out that the observed effects are due to differences in expression.

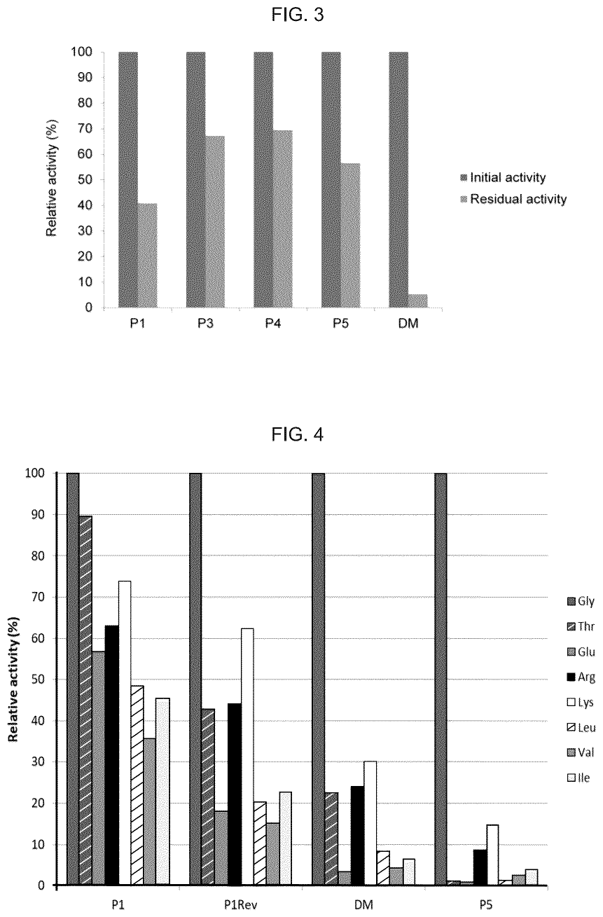

In order to select the best option for FuncLib, the thermostability was evaluated (FIG. 3). All of the mutants tested were more thermostable than DM.

All of the variants were tested for activity. P1 outperformed DM but the P5 mutant resulted to be the worst candidate compared to P1. The P1 mutant was cloned again without the S153N mutation (P1 rev) and we assayed the variants with the different substrates where the G in SEQ ID NO:19 following the TEV recognition sequence was varied and replaced by other amino acids as indicated (FIG. 4). P1 rev was not as good as P1 but it was much better than DM. P5 was not the best candidate according to its activity in comparison with other variants, so we tested P1 rev thermostability to evaluate if it was stable enough for further rational optimization.

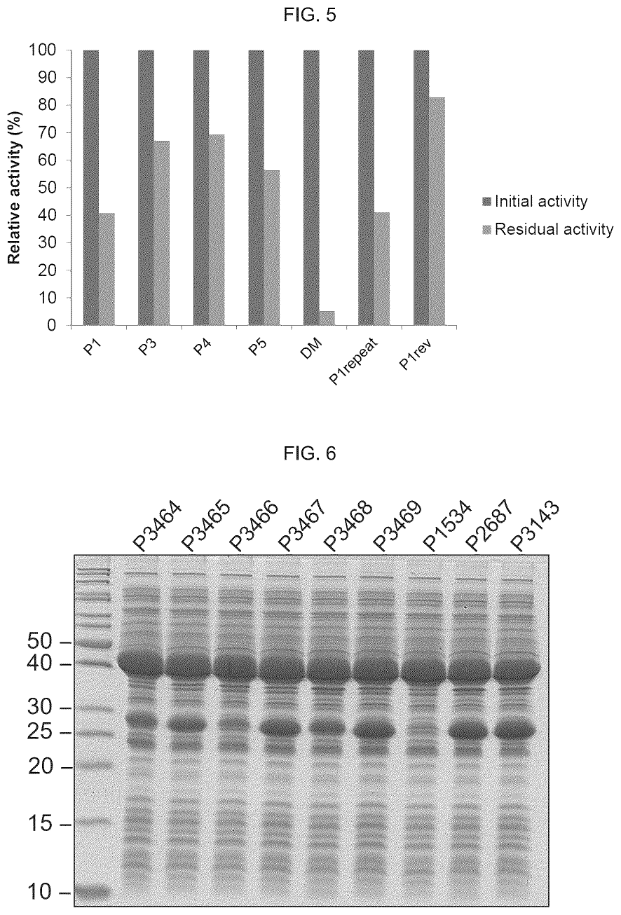

Thermostability results showed that P1 rev outperformed the rest of the variants, including patent protected DM, presenting P1 rev as the best candidate further optimization (FIG. 5). This mutant was more stable, with a less selective activity profile. According to further analysis of the mutant, it seems that mutations have allowed stabilization of the loop through new polar contacts within the amino acids in the surroundings, allowing the thermostability change and possibly causing an overall stabilization of the protein leading to an improvement in activity.

The experiments conducted allowed to obtain a variant with higher stability than DM and standard TEVp Said mutant was found to have better activity with the fluorogenic modified substrates and better thermal stability, making it an ideal candidate for future protein engineering studies.

TABLE 2
Construct overview for Examples 3 to 5

| | SEQ | | | | | | |
|---|---|---|---|---|---|---|---|
| | ID NO: | | | | | | |
| P3464 | 24 | Q74L | S153N | R203G | | | |
| P3465 | 25 | S135G | S153N | R203G | | | |
| P3466 | 26 | I138T | S153N | R203G | | | |
| P3467 | 27 | Q74L | S135G | S153N | R203G | | |
| P3468 | 28 | Q74L | I138T | S153N | R203G | | |
| P3469 | 29 | S135G | I138T | S153N | R203G | | |
| P1534 (DM) | 3 | S153N | R203G | | | | |
| P2687 (P1) | 13 | Q74L | S135G | I138T | S153N | R203G | |
| P1-M218F | 30 | Q74L | S135G | I138T | S153N | R203G | M218F |
| (P3143) | | | | | | | |

### Example 3: Analysis of P1 Mutations Q74L, S135G, I138T

### Expression Analysis:

All constructs were expressed in small-scale expression studies and protein production was induced by addition of arabinose. The following SDS-PAGE gel shows the cell pellets 16 hours after protein expression induction (FIG. 6). In addition to the mutations indicated in Table 2, all constructs have the sequence set forth in SEQ ID NO:1, i.e. additionally comprise the substitutions 17S, 68D, 77V, 219N, 153N and 203G relative to wildtype TEV protease (SEQ ID NO:2).

### Cell Lysate Analyses and Activity Test:

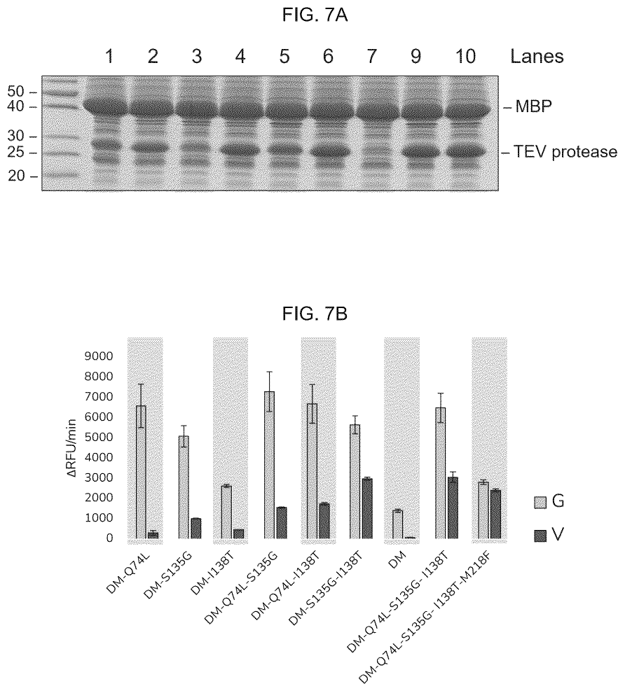

The obtained cell pellets after expression studies were lysed by BugBuster Kit (Merck Millipore). The resulting cell lysates were diluted 10-fold in PBS before loaded on SDS-PAGE gel for analysis (FIG. 7A). Furthermore, the TEV protease activity in the diluted cell lysates were assessed by fluorescence assay employing fluorogenic substrates with G and V in the P1′ position (FIG. 7B).

### Results:

The experiments demonstrate that the mutations Q74L, S135G and I138T affect the TEV protease's solubility and activity. In terms of stability, varying thickness of protein bands indicate that different amounts of TEV protease were recovered after cell lysis (FIG. 7A). Both mutations, S135G and Q74L play a major role in enhancing the TEV protease solubility, while the I138T led to only minor solubility improvements (FIG. 7A, compare lanes 1, 2, 3 with 7). The combinations Q74L-S135G and S135G-I138T lead to synergistic effects and further enhance the stability of the protease (FIG. 7A, compare lanes 4 and 6 with 1, 2, 3). Both double mutants contain the S135G mutation indicating that it is an important mutation for TEV protease solubility. The solubility of the TEV protease with the mutations Q74L-I138T is comparable to that of single mutants Q74L and S135G.

Furthermore, the triple mutant including Q74L, S135G, and I138T (also referred to as P1 TEV protease) exhibit excellent stability comparable to that of Q74L-S135G and S135G-I138T (FIG. 7A, see lanes 9 in comparison to 4 and 6). The data demonstrate that maximum solubility does not require all three mutations, but two specific mutations, either Q74L-S135G or S135G-I138T are sufficient for this purpose.

In terms of activity, different solubility properties of variants resulted in varying amounts of enzyme used in the TEV protease activity assay. TEV protease recovery after cell lysate preparation seem to be comparable in lanes 1, 2, 5 (group 1) and 4, 6, 9, 10 (group 2), which makes comparison of the activity within the groups possible. The data indicate that under the applied conditions the I138T mutation enhances the activity against V in P1′ (FIG. 7B, compare lane 4 and 9, or lane 1 and 5). The mutation combinations S135G/I138T lead to synergistic effects further increasing the activity against V (FIG. 7B, compare lane 6 with 4/5), while the Q74L mutation does not seem to affect the activity of TEV protease against V and G (FIG. 7B, compare lane 6 with 9). In terms of stability and also activity against amino acids G and V at P1′, the S135G-I138T mutant performs similar as the triple mutant Q74L-S135G-I138T indicating that the Q74L mutation may have little effect on these properties. In contrast, mutations S135G and I138T seem to be important in that they enhance TEV protease solubility and activity. In this example, the M218F mutation seem to considerably decrease the activity of the TEV protease activity against G compared to P1 (Km values P1 not determined) (FIG. 7B, compare 9 with 10).

### Example 4: Analysis of P1, P1-M218F and DM Against Fluorogenic Peptide Substrates

### Overview of TEV Protease Variants:

P1: SEQ ID NO:1 with Q74L, S135G, I138T, S153N and R203G
P1-M218F: SEQ ID NO:1 with Q74L, S135G, I138T, S153N, R203G and M218F
DM: SEQ ID NO:1 with R203G, S153N

All TEV protease variants were purified by IMAC and IEX chromatography and their activities were assessed in fluorescence-based activity assays. All eight synthetic peptide substrates used in the assay contain the peptide sequence ENLYFQXGGK (where X represents the residues G, T, K, L, R, E, V and I) (SEQ ID NO:22). The peptides were labelled with an EDANS fluorophore and a Dabcyl quencher at the termini and were synthesized by Pepscan (Netherlands). Upon TEV protease cleavage (between Q and X) the EDANS fluorophore is released and the fluorescence intensity, referred to as relative fluorescence unit (RFU) can be measured at Ex/Em=340/490 nm. The RFUs of cleavage reactions were measured every 30 sec at 30° C. for 4 h (FLUOstar OPTIMA, BMG Labtech). The RFU was plotted against different EDANS concentrations (EDANS standard curve). Two time points (T1 and T2, ΔT) in the linear range of the cleavage reaction plot were chosen and the amount of released EDANS molecule (B, in pmol) was calculated based on the respective fluorescence values (RFU2 and RFU1, ΔRFU) and EDANS standard curve. The initial velocities related to 1 mg enzyme (specific activity) for 25 μM substrate were calculated based on the quotation below:

$$Specific activity = \frac{B}{Δ T \times M} \times Dilution factor B$$

B: amount of product calculated from EDANS standard curve
ΔT: linear reaction time T2−T1 (min)
M: amount of the protein in the sample (mg)

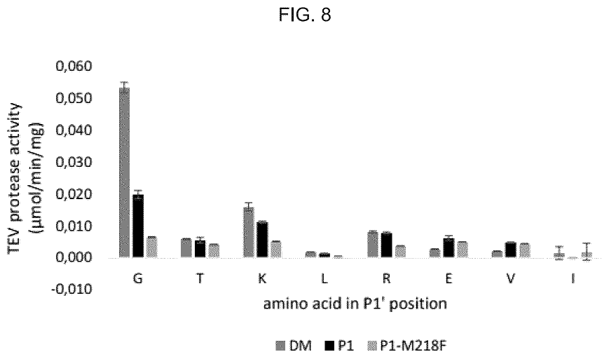

The specific activities of DM, P1 and P1-M218F against 25 μM of all eight synthetic peptide substrates are shown in FIG. 8. In experiments independent of these studies, the Km and vₘₐₓ values of the DM and P1-M218F TEV variants against all eight substrates were determined and are listed below in Table 3.

TABLE 3
Km vₘₐₓ values of the DM and P1-M218F TEV variants determined
derived from Michaelis Menten Kinetic studies for all eight
synthetic peptide substrates (attention: Km and vₘₐₓ values
of P1 not determined so far)

| Synthetic peptide | | | | |
|---|---|---|---|---|
| substrates with | | | | |
| different amino | DM | | P1-M218F | |
| acids at P1′ | Km | vₘₐₓ | Km | vₘₐₓ |
| position | (in μM) | (nmol/min/mL) | (in μM) | (nmol/min/mL) |
| G | 13.7 | 56.0 | 2.9 | 7.5 |
| T | 7.0 | 4.0 | 1.1 | 2.3 |
| K | 13.8 | 5.0 | 7.4 | 2.1 |
| L | 23.2 | 4.8 | 2.2 | 1.7 |
| R | 47.2 | 17.6 | 14.5 | 4.1 |
| E | 41.0 | 8.9 | 1.5 | 4.8 |
| V | 45.8 | 3.8 | 2.5 | 3.3 |
| I | 45.9 | 2.7 | 5.3 | 2.3 |

### Results:

Table 3 shows that the Km values of P1-M218F against all substrates were reduced compared to DM (ranging between 1-15 μM), so by applying 25 μM substrate in the reaction it can be assumed that the variant operated in substrate excess (around 2 to 25-fold). The Vₘₐₓ values are relatively low (1.7-4.8 nmol/min/mL), i.e. small substrates concentrations are sufficient to ensure that the enzyme works at maximum speed.

However, the Km values of DM, in particular for L, R, E, V and I substrates were relatively high (around 40 μM), i.e. in case of the DM variant higher concentrations would have been required to achieve substrate saturation and thus ensure maximal cleavage activity. As the product formation or i.e. the protease activity highly depends on the substrate availability the data obtained with DM against L, R, E, V and I substrates should be viewed with caution. As for DM against G substrate the Km value is 13.7 μM and the vₘₐₓ is 3-20-fold higher than the vₘₐₓ values of other substrates.

Nonetheless, it can be concluded that the specific activity of the DM variant against 25 μM of G and K substrates is considerably higher than of the P1 (around 3-fold) and P1-M218F (around 8-fold) variant (FIG. 8). The data indicate that the M218F mutation seem to decrease the activity of the TEV protease against G as already implied by data shown in FIGS. 7A-7B. P1 and P1-M218F exhibit comparable specific activities against substrates containing I, V, E, R, L, and T at P1′. In general, the specific activities of P1 seem to be higher against G and K at P1′ than of P1-M218F.

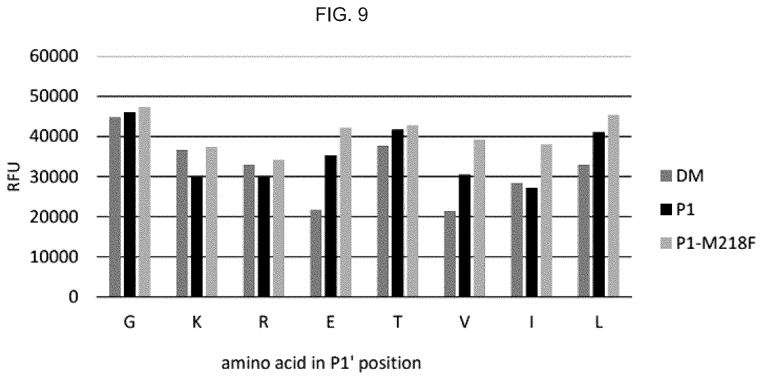

After 4 hours of incubation, comparable fluorescence signals (i.e. cleavage efficiency) were observed for all three variants against G, K, R, and T (FIG. 9). P1 and P1-M218F exhibited higher cleavage efficiencies against hydrophobic amino acids (E, V, and L) compared to DM. The M218F mutation appeared to enhance activity specifically against E, V, I, and L, as the fluorescence signal of P1-M218F was higher than that of P1.

In terms of catalytic activity against substrates, the P1-M218F TEV protease appears to be slower than the DM and P1 variants (FIG. 8, e.g. G, T, K, R), but overall it achieves higher cleavage efficiencies, indicated by the higher RFU values achieved for P1-M218F against all eight substrates compared to DM and P1 (FIG. 9).

### Example 5: Analysis of P1, P1-M218F and DM TEV Protease Activity Against Fusion Protein Substrates

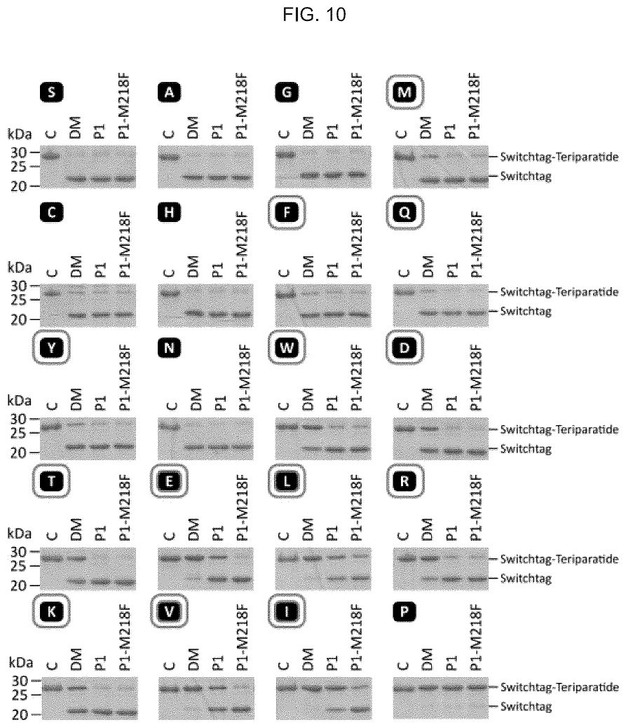

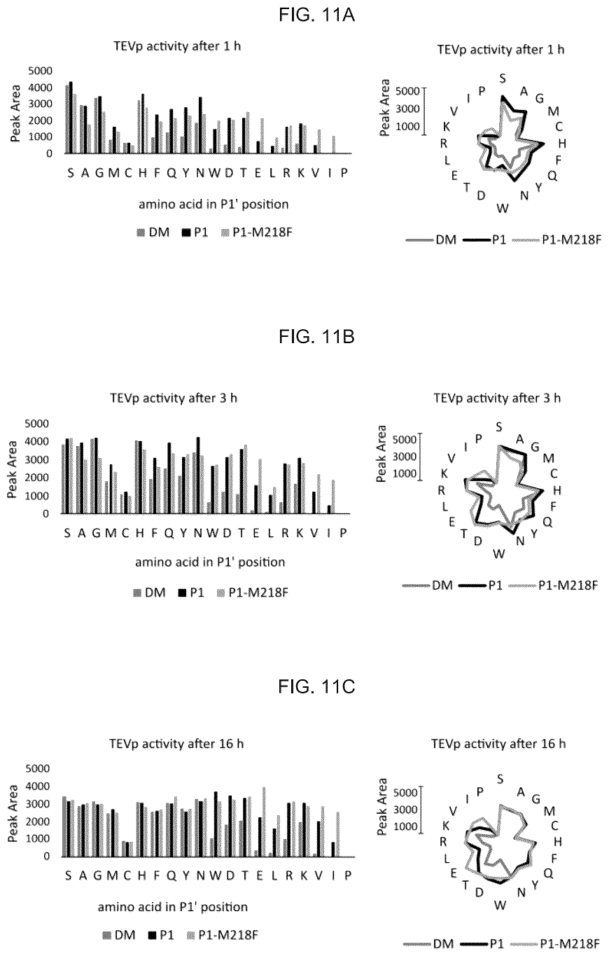

All three TEV protease variants were tested against twenty proteogenic Switchtag-Teriparatide (Switchtag is a HlyA fragment, 165 aa in length; Teriparatide is a recombinant human PTH fragment consisting of aa 1-34 of PTH (CAS No. 52232-67-4)) substrates containing the TEV protease recognition site (ENLYFQ) (SEQ ID NO:34) between the Switchtag and the Teriparatide part, each of them harboring a different canonical amino acid residue at P1′. TEV protease variants were added to 25 μM of Switchtag-Teriparatide substrates in a molar enzyme to protein ratio of 1:25 and cleavage reactions were incubated at 30° C. SDS-PAGE samples of cleavage reactions were prepared after 16 h of incubation (FIG. 10). HPLC samples of cleavage reactions were prepared after 1 h, 3 h and 16 h of incubation (FIGS. 11A-11C).

### HPLC Analysis:

For HPLC analysis the cleavage samples were immediately quenched with 1:1 (v/v) 6 M GuHCl after sampling and 80 μL of the mixture was injected into Agilent HPLC system for RP-HPLC analysis. The components were eluted by a linear gradient of acetonitrile (5-60%) in water with 0.1% (v/v) TFA and elution signals of released Teriparatide were integrated by the OpenLab ChemStation data software (Agilent). The bar charts in FIGS. 11A-11C, left panel, show the peak areas of released Teriparatide after elution peak integration. For better visualization of the substrate specificity, the data were additionally presented in a network diagram.

### Results:

The cleavage efficiencies of DM, P1 and P1-M218F against amino acids such as S, A, G, M, H, F, Q, Y and N were comparable (FIG. 11C). However, when it comes to the remaining amino acids (W, D, T, E, L, R, K, V, 1), the P1 and P1-M218F variants demonstrated superior performance compared to DM. While both P1 and P1-M218F showed similar cleavage capabilities against the amino acid residues W, D, T, R, and K at P1′, P1-M218F outperformed P1 when it comes to hydrophobic amino acids such as L, V, and I and the negatively charged residue E. This is clearly attributable to the M218F mutation and the data correlated with data obtained in FIG. 9. Furthermore, the HPLC data in FIGS. 11A-11C aligned well with the SDS-PAGE data in FIG. 10 except for the C substrate. According to SDS-PAGE analysis, about 80% of the Switchtag-Teriparatide (for C at P1′) substrate was cleaved, but Teriparatide recovery after HPLC analysis was considerably low (roughly 20%) (FIGS. 11A-11C). Most likely, proper product recovery is hampered by formation of cysteine dimerization during cleavage reaction, but further studies are currently conducted to resolve this.

### Example 6: Rational Engineering of a TEVp Variant with Improved Activity Towards Unfavored Residues at the P1′ Position

### Methods

### SenseNet

Starting from a known TEV protease (PBD ID: 1LVM) mutations T17S, N68D, I77V, R203G, S219N were introduced, the mutation C151A was reversed, and residues 237-242 were removed to obtain the SM variant (SEQ ID NO:31). Then a section of a Teriparatide-HlyA fragment fusion construct containing a GGSENLYFQSVS linker sequence (SEQ ID NO:35) with the substrate in 1 LVM was aligned. The P1'S was then mutated to a V.

### High-Throughput Screening Protocol for TEV Protease

### Cell Culture

Mutagenic library was transformed to the BL21 E. coli competent cells using pRIL as co-expression plasmid. Individual clones were picked and inoculated in sterile 96-well plates (Greiner Bio-One, GmbH, Austria), referred to as master plates, containing 100 μL of LB media, supplemented with 34 μg/mL Chloramphenicol+30 μg/mL Kanamycin, per well. Plates were sealed with parafilm and incubated at 37° C., 250 rpm and 80% relative humidity in a humidity shaker (Minitron, Infors, Switzerland) overnight. In each plate, column 6 was inoculated with the parental type single mutant (SM), column 7 was inoculated with double mutant (DM) and one well (H1-control) was inoculated with p8ref (TEV protease without activity). After 24h incubation, plates were replicated with cryo-replicator into new 96-deep well plates containing 300 μl of TB media supplemented with 34 μg/mL Chloramphenicol+30 μg/mL Kanamycin and 2 mM MgCl₂. Plates were incubated at 37° C., 250 rpm and 80% relative humidity.

When OD₆₀₀ reached 0.6-0.8, in each well was added 100 μL of TB media supplemented with 34 μg/mL Cm+30 μg/mL Km+2 mM MgCl₂ and IPTG in a final concentration of 0.1 mM. Plates were further incubated for 19 h 250 rpm, 80% relative humidity at 25° C.

### Cell Disruption

Plates were centrifuged (Eppendorf 5810R centrifuge, Germany) for 30 min, 3500 rpm at 4° C. and supernatant was discarded. In each cell pellet 150 μL of BugBuster solution was added using multi-drop dispenser (Multidrop Combi Reagent Dispenser, Thermo Scientific, USA) and plates were shaken in a plate shaker until pellet was resuspended. Finally, plates were incubated in humidity shaker for 20 min, 25° C., 250 rpm and centrifuged for 40 min, 3500 rpm at 4° C. to obtain the lysate solution.

### Screening Assay

20 μL lysate was transferred from the 96-deep well plate using robotic station (Freedom EVO Tecan, Switzerland) to a microplate already containing 80 μL of high purity water (1:5 dilution). Afterwards, from 1:5 dilution microplate, 50 μl was transferred by the robot to a new microplate containing 50 μl of assay buffer 2× (100 mM Tris-HCl, 1 mM EDTA) (1:10 dilution). Next, 25 μl of 1:10 dilution was transferred to empty opaque microplates, 25 μl of previously prepared substrate solution was added and fluorescence (Ex/Em=340/490) was measured immediately in plate reader (SPECTRAMax Plus 384, Molecular Devices, USA). Substrate solution of each peptide was composed of 50 mM Tris-HCl pH8.0, 0.5 mM EDTA, 1 mM DTT and 10 μM of corresponding fluorogenic peptide. The values were normalized against the SM and DM of the corresponding plate.

### Rescreening

To rule out the selection of false positives, plasmids were extracted from winner clones and BL21 E. coli was transformed again as described before. Eight individual colonies of each selected clone were inoculated in sterile 96-well plates. Again, for reliable comparison, column 6 was inoculated with the parental type SM, column 7 was inoculated with double DM. Culture growing and screening assay were done as described before.

### Expression of TEV Protease Variants

Pre-cultures of E. coli BL21 cells transformed with plasmids containing the genes of the SM and clone 1 TEVp variant were grown from 5 μL cryoculture in 25 mL 2YT medium containing 100 μg/mL ampicillin for selection in 250 mL shaking flasks. The cultures were cultivated at 37° C. and 180 rpm overnight.

On the next day, 1 L of selective 2YT medium in 5 L shaking flasks were inoculated with pre-cultures to initial OD₆₀₀ of 0.1 in 5 L shaking flasks. The cell cultures were grown at 30° C. and 180 rpm until OD₆₀₀ of 0.4-0.6 were reached and protein expression was induced with 0.1 M IPTG. The cells were cultivated at 25° C. and 180 rpm overnight and harvested by centrifugation at 16,000 g and 4° C. for 30 min (Avanti JXN-26, Beckman Coulter GmbH).

The cell pellets were resuspended in 50 mM Tris/HCl pH 7.3, 250 mM NaCl, 0.5 mM EDTA, 5 mM DTT, 1 mM PMSF, 5% (w/v) glycerol and disrupted by high pressure homogenization (LM-20, Microfluidics) for three cycles at 1,200 bar. Crude cell lysates containing soluble TEV protease were obtained after centrifugation at 16,000 g and 4° C. for 20 min.

### Purification of TEV Protease Variants

Crude cell lysates were supplemented with 1 mM imidazole and both TEV protease variants were purified by immobilized metal affinity chromatography (IMAC) using the AKTA™ pure chromatography system. The cell lysates supplemented with 1 mM imidazole were loaded onto a cOmplete His-Tag purification column (Roche) pre-equilibrated in 50 mM Tris/HCl pH 7.3, 250 mM NaCl, 0.5 EDTA, 5 mM DTT, 5% (w/v) glycerol, 1 mM imidazole. Bound His₆-tagged TEV proteases were eluted in a step gradient with 30% buffer B which corresponds to buffer containing 300 mM imidazole. The elution signals were monitored by UV absorbance at 280 nm. Elution fractions containing TEV protease were pooled, the buffer was exchanged for a buffer with low salt content (50 mM Tris/HCl pH 7.3, 0.5 mM EDTA, 5 mM DTT, 5% (w/v) glycerol) by gel filtration (HiPrep 26/10 Sephadex G-25, Cytiva). Remaining impurities were separated from the TEV protease by cation exchange chromatography (CEX) employing the Capto SP ImpRes column (Cytiva) pre-equilibrated in 20 mM Tris/HCl pH 7.5, 1 mM EDTA, 5 mM DTT, 5% (w/v) glycerol. Bound TEV proteases were eluted by a step gradient with 10% buffer B which corresponds to buffer containing 100 mM NaCl. The elution signals were monitored by UV absorbance at 280 nm.

### TEV Protease Activity Against Fluorogenic Peptide Substrates

Purified SM and clone 1 TEVp variants were tested against eight fluorogenic peptide substrates to assess their cleavage efficiency against various amino acid residues in the P1′ position. All eight synthetic peptide substrates contained the peptide sequence ENLYFQXGGK (SEQ ID NO:22) (where X is represented by the residues G, K, R, E, I, V, T and L). The peptides were labelled with an EDANS fluorophore and a Dabcyl quencher at both ends and were synthesized by Pepscan (Netherlands). TEV protease cleavage between X and G in the peptide lead to release of the EDANS fluorophore and the fluorescence intensity can be measured at Ex/Em=340/490 nm.

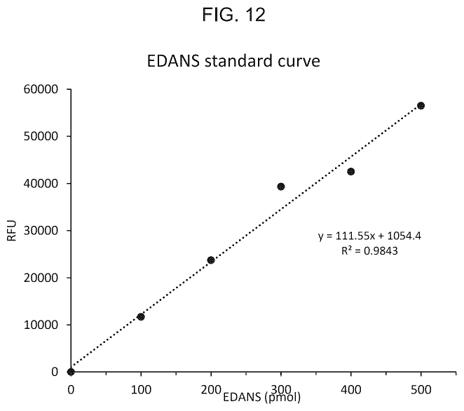

Lyophilised fluorogenic peptides were reconstituted in DMSO to obtain 1 mM stock solutions. Each substrate mix contained 50 μM peptide in reaction buffer (50 mM Tris/HCl pH 8.0, 0.5 mM EDTA, 80 mM DTT). Purified SM and clone 1 TEVp variants were diluted to 30 μg/mL in 100 mM Tris/HCl pH 8.0, 1 mM EDTA and 25 μL of diluted TEVp was added to 25 μL substrate mix in black 96-well microtiter plates. The relative fluorescence unit (RFU) of the samples were measured every 30 sec at 30° C. for 2 h (FLUOstar OPTIMA, BMG Labtech). The RFU was plotted against different EDANS concentrations (FIG. 12). Two time points (T₁ and T₂, ΔT) in the linear range of the plot were chosen and EDANS release (B, in pmol) was calculated based on the respective fluorescence values (RFU₂ and RFU₁, ΔRFU) and EDANS standard curve. The TEVp activities per mg enzyme were calculated for each TEVp variant and fluorogenic peptide substrate based on the following formula:

$$TEVp activity per mg enzyme = \frac{B}{Δ T \times M} \times DF = \frac{p mol}{min} / mg$$

B=EDANs amount calculated from EDANS standard curve
ΔT=T₂−T₁, time points in the linear range
M=Amount of enzyme in the reaction
DF=sample dilution factor
   TEV Protease Activity Assays with Fusion Protein Substrates Purified SM and clone 1 TEVp variants were tested against twenty Switchtag-Teriparatide fusion protein substrates containing the TEV protease recognition site (ENLYFQ) (SEQ ID NO:34), each of them harboring a different canonical amino acid residue in the P1′ position. Genes encoding Switchtag-Teriparatide fusion proteins were cloned in the parental plasmid of HlyA1 (Khosa, S. et al. An A/U-Rich Enhancer Region Is Required for High-Level Protein Secretion through the HlyA Type I Secretion System. Appl. Environ. Microbiol. 84, (2018)). and transformed into E. coli BL21 (DE3) cells. Intracellular expression of Switchtag-Teriparatide as IBs, IB extraction and denaturation in 6 M GuHCl were performed as described in Nguyen et al. (2021) (Nguyen, B.-N. et al. Numaswitch: an efficient high-titer expression platform to produce peptides and small proteins. AMB Express 11, 48 (2021)). The protein concentrations of the solubilized IBs were determined by UV/Vis spectroscopy at 280 nm (NanoDrop™ One/One C, ThermoFisher) using the calculated molecular weights and extinction coefficients (ProtParam, Expasy). Renaturation of Switchtag-Teriparatide fusion proteins was performed by rapid dilution in Tris/HCl buffer (50 mM, pH 8.0, 150 mM NaCl, 10 mM CaCl₂, 0.5 mM EDTA) and the protein concentration of each substrate was adjusted to 1 mg/mL. Purified SM and clone 1 TEVp variants were added to refolded Switchtag-Teriparatide substrates in a molar enzyme to protein ratio of 1:25, respectively. The cleavage reactions were incubated at 30° C. for 1 h and 24 h. After incubation, the cleavage reactions were immediately quenched with 1:1 (v/v) 6 M GuHCl and 80 μL of cleavage samples were analyzed by RP-HPLC analysis using the 1260 Infinity II HPLC (Agilent) system and a ZORBAX SB-C18 column (80 Å, 5 μm, 4.6 mm×250 mm). The components were eluted by a linear gradient of acetonitrile (5-60%) in water with 0.1% (v/v) TFA at a flow rate of 1 mL/min. The elution signals were monitored by UV absorbance at 205 nm. Chromatographic elution peaks of released Teriparatide after proteolytic cleavage were integrated by the OpenLab ChemStation data software (Agilent). For visualization of differences of both TEVp variants the values were normalized to the highest substrate protein concentration used in cleavage reactions and the relative cleavage efficiencies were compared to SM TEVp with ENLYFQ-S(SEQ ID NO:36) (set to 100%). Cleavage reactions comprising the ENLYFQ-S substrate were additionally analyzed by RP-HPLC-MS to confirm the identity of released target in the elution peak. The samples were injected into an Alliance™ HPLC system coupled to an electron spray ionization (ESI) source and a quadrupole mass spectrometer (MS, ACQUITY QDa Detector, Waters). The components were separated as described above. Following ESI in positive ion mode and quadrupole analysis the molecular masses of the elution signals were analyzed using the Empower 3 chromatography data software.

### Mechanistic Studies

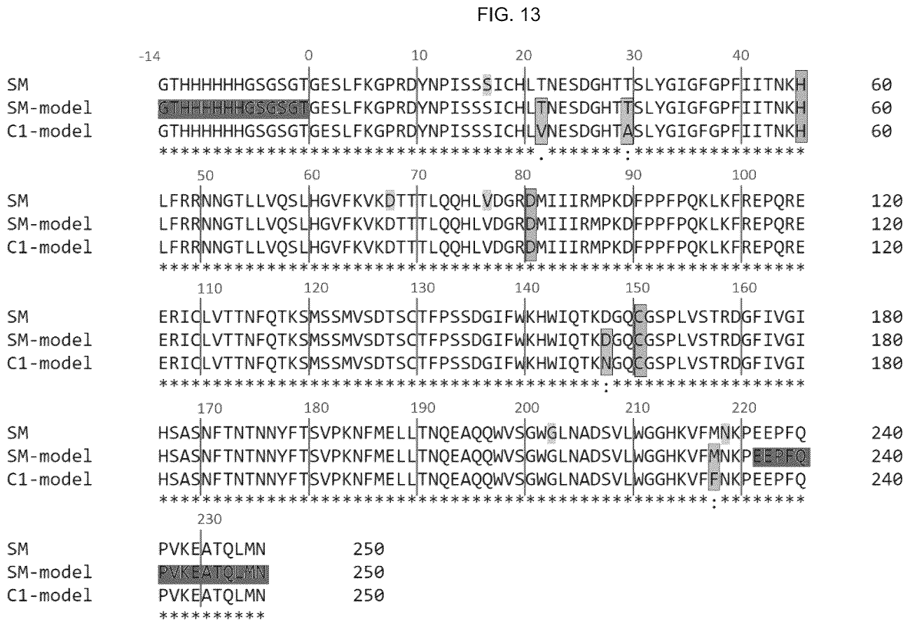

System setup. A crystal structure of inactive TEV protease, in its C151A variant, complexed with TENLYFQSGT substrate (SEQ ID NO:37) (PDB ID: 1LVB) (Phan, J., Zdanov, A., Evdokimov, A. G., Tropea, J. E., Peters III, H. K., Kapust, R. B., Li, M., Wlodawer, A., Waugh, D. S. “Structural basis for the substrate specificity of tobacco etch virus protease.” (2002) J Biol Chem 277: 50564-50572. doi.org/10.1074/jbc.M207224200), was used to prepare two models of the SM and Clone-1 (C1) variants in complex with two fluorogenic peptides, ACE-E(EDANS)-NLYFQ-GGK-DABCYL (SubG) (SEQ ID NO:38) and ACE-E(EDANS)-NLYFQ-VG-K(DABCYL) (SubV) (SEQ ID NO:39) as used in the experiments. For this purpose, a mutation from Ala to Cys in position 151 was reversed, and subsequently missing N-termini amino acids (highlighted in bold) GHHHHHHGESLFKG-⁸PRD (SEQ ID NO:40) were added using as a template the structure of the TEV protease complexed with a product (PDB ID: 1 LVM). The N-termini has then been modified from GHHHHHH-¹GESLFKG-⁸PRD (SEQ ID NO:40) to GTHHHHHHGSGSGT-¹GESLFKG-⁸PRD (SEQ ID NO:41). Subsequently, missing amino acids at C-termini (again highlighted in bold) -NKP²²¹-EEPFQPVKEATQLMN (SEQ ID NO:42) were added using the Discovery Studio ver. 21.1.0.20298 program (BIOVIA, Dassault Systemes, Discovery Studio 2021). The improved TEV protease structure model was used to generate the SM variant by introducing five mutations, i.e., T17S, N68D, I77V, R203G, and S219N. All modifications in the sequence of the initial crystal structure are highlighted in FIG. 13.

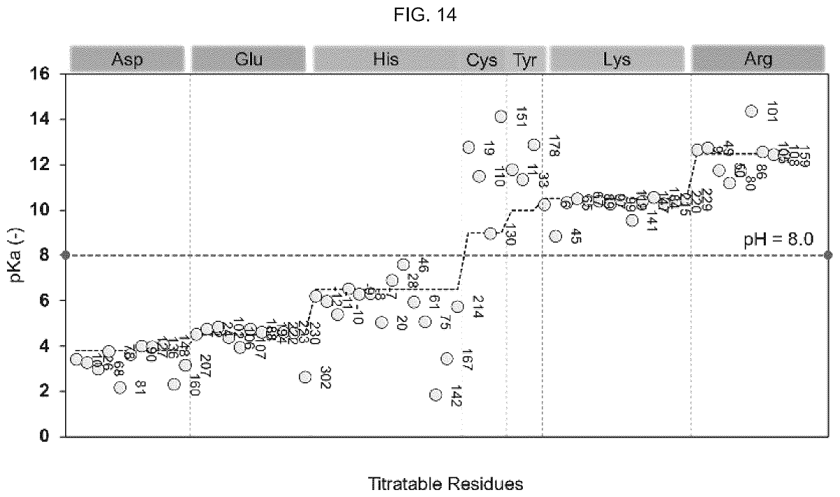

The pKa shift of all titratable residues was determined with PropKa software ver. 3.1. As shown in FIG. 14 all residues were found in their natural protonated state at pH 8.0. After geometrical inspection, histidine residues in positions −12, −11, −10, −9, −8, −7, 20, 61, 75, 142, and 167 were protonated in ε-, while His28, and His214 in δ-position. Disulfide bridges were not detected in the structure. Catalytic His46 was found with a slightly elevated value of pKa (7.58) but considering that experiments were conducted at pH 8.0 and that the pKa of Cys151 is 14.12 the His41 was deprotonated at NE position. Therefore, it was assumed that in the case of this cysteine protease, the active site adapts its neutral form. The protonation position was additionally verified by visual inspection of the orientation of the Cys151-His46-Asp81 triad in the active site of the crystal structure of the wild-type TEV protease.

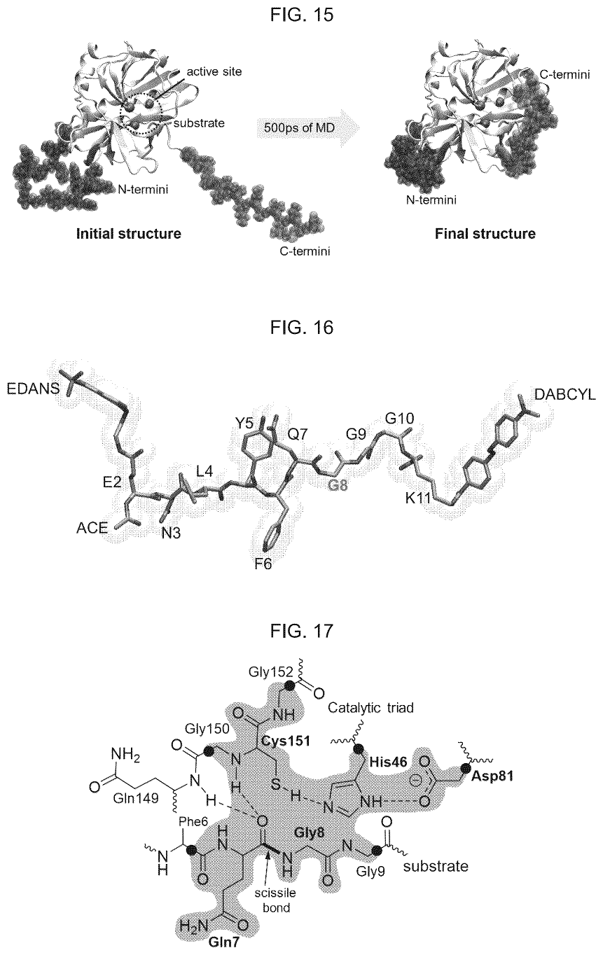

Missing hydrogen atoms were added to the SM enzyme and substrate using the tLEAP (Schafmeister, C. E. A.; Ross, W. S.; Romanovski, V. LEAP, University of California, San Francisco, 1995) module of the AmberTools package. Insertion of residues at both ends of the protein resulted in extended peptide chains that required optimizing their position to adapt to the rest of the protein structure. Therefore, pre-equilibration involving minimization and 500 ps of NVT molecular dynamic (MD) simulations were done in the gas phase at molecular mechanics (MM) level using the AMBER ff03.r1 force field. To keep the crystal structure kernel unchanged, only residues belonging to the added termini parts were allowed to move during simulations. The initial structure and one obtained after simulations are shown in FIG. 15. The presence of the C-end chain is especially critical because these originally missing residues seem to be involved in the formation of the binding pocket of the substrate.

The pre-optimized model of SM was used to build the C1 model that was prepared by the insertion of four additional mutations, T22V, T30A, D148N, and M218F as predicted by SenseNet analysis. The structure of fluorogenic peptide was prepared based on the positions of the atoms of the original substrate present in the crystal structure. As indicated in FIG. 16 both fluorophore (EDANS), as well as quencher (DABCYL) groups were attached to side chains of Glu and Lys residues, respectively. Missing AMBER force field parameters for these atypical residues were generated employing Generalized Amber Force Field (GAFF) and the atomic charges were computed using the AM1 method with bond charge corrections (AM1-BCC). Parameters and atomic charges were generated using the Antechamber software (data not shown).

In the case of the SM model, the overall charge of the SM:SubG and SM:SubV complexes was 0, therefore counterions were not added to the system. In the C1 model, the mutation of D148N caused the appearance of a positive charge that has been neutralized by the addition of one chloride (Cl—) counterion that was placed in the most electrostatically favorable position. Subsequently, the system was soaked within an orthorhombic box of TIP3P water molecules, with an average size of 79×79×74 Å³. To describe the protein and water molecules the AMBER ff03.r1 and TIP3P force fields, respectively, were employed and the NAMD software was used as a molecular dynamic (MD) engine. A cut-off for non-bonding interactions was set between 14.5 to 16 Å using a smooth switching function. The temperature during the simulations was controlled using the Langevin thermostat, and the pressure with the Nosé-Hoover Langevin piston pressure control. In all simulations, periodic boundary conditions (PBC) were applied.

MD simulations. The equilibration protocol for MD simulations of four prepared models (SM:SubG, SM-SubV, C1:SubG, and C1:SubV) involved a preliminary minimization and gradual heating of the system to 303.15 K with 0.001 K temperature increments, followed by 100 ps of non-biased NPT equilibration. The 6 μs of no-restricted unbiased NPT MD simulations with the SHAKE algorithm used to restrain all hydrogen bonds with a 2 fs time step were done. Due to the possible bias of starting point generated by introduced mutations and missing protein fragments, the first 2 μs of MD simulations were dedicated to system equilibration, and therefore the results from the last 4 μs of simulations were considered during the analysis of the results. The last structures generated during the long MD simulations were subsequently used as initial structures for a reaction mechanism study carried out by means of a hybrid quantum mechanics/molecular mechanics (QM/MM) approach.

QM/MM calculations. To further analyze the effect of the proposed mutation on TEV capacity to catalyze the cleavage of the peptide bond formed between Gln and Gly as well as Gln and Val residues, the reaction mechanisms were computationally explored in SM-SubG, SM-SubV, and C1-SubV variants. The AMBER and TIP3P force fields, as implemented in the fDynamo library were used for describing the protein, counterions, and water molecules. The cut-off scheme for non-bonding interactions was the same as the one used in the classical MD simulations protocol. Additionally, the positions of the atoms of residues beyond 20 Å from the inhibitor were fixed. The QM part of the model was selected including only the essential 66 atoms in the case of E:SubG and 75 atoms of E:SubV models, as shown in FIG. 17. These include part of the substrate, Cys151 with part of neighboring Gly150 and Gly152, as well as the side chain of His46 and Asp81. Six-link atoms (Field, M. J.; Albe, M.; Bret, C.; Martin, F. P.-D.; Thomas, A. The dynamo library for molecular simulations using hybrid quantum mechanical and molecular mechanical potentials. J. Comput. Chem. 2000, 21, 1088-1100) were inserted on the QM/MM boundaries, placed in the Ca-Cp bond of His46 and Asp81, the C-Cα bond in the case of Gly150, Gly151 of protein, and for Phe6 and Gly9 in the substrate. The Austin Model, AM1 semiempirical Hamiltonian, and the Minnesota density functional, M06-2X with the standard 6-31+G(d,p) basis set, were employed to treat the QM sub-set of atoms, as detailed below.

Potential Energy Surfaces (PES). Distinguished reaction coordinates, ξ, were selected for exploring each chemical step of the peptide bond cleavage mechanism with SubG and SubV. Each PES was generated by grid scanning, where the step size for hydrogen transfer was controlled every 0.05 Å, while distances between heavy atoms were changed by 0.1 Å. Computed PES allowed identifying the minimum energy path (MEP). All stationary points observed along the MEP were optimized at low and high level of theory (AM1/AMBER and M06-2X/AMBER, respectively) employing a micro-macro iteration method using Baker's algorithm and they were characterized by computing the matrix of the second energy derivatives. All TSs computed at M06-2X/MM (Cartesian coordinates for QM atoms are not shown) were connected to the expected minima by tracing intrinsic reaction coordinate (IRC) paths, to further confirm the explored inhibition mechanism.

Free Energy Surfaces (FES). Series of QM/MM MD simulations employing the umbrella sampling (US) method as implemented in fDynamo, were computed using previously generated structures of the AM1/MM PESs. A force constant of 2500 kJ·mol⁻¹·Å⁻² was used to constrain the reaction coordinate and 303.15 K was set as the simulation temperature. An initial equilibration of 5 ps was carried out, followed by 20 ps of production at every window. Finally, the weighted histogram analysis method (WHAM) was used to integrate the obtained results in terms of potentials of mean force (PMF). A density tolerance of 10⁻³ to consider the WHAM calculation converged. The PMF was obtained as a function of the distinguished reaction coordinate as described in detail in previous studies carried out in our laboratory.

Spline Corrections. To improve the quality of obtained results and to reduce the possible errors associated with the semiempirical method used during free energy simulations, high-level corrections were employed using DFT. Spline corrections were applied through the energy function

$$E = {E}_{LL / MM} + S [ Δ {E}_{LL}^{HL} ( {ξ}_{1} , {ξ}_{2} ) ]$$

where the final energy is obtained from a correction term computed using the single-point energy difference between the high-level (HL) and the low-level (LL) for the QM sub-set of atoms. As mentioned above, the AM1 method was applied as LL method while as the HL the hybrid M06-2X functional with the standard 6-31+G(d,p) basis set was used. The Gaussian 09 program combined with fDynamo was employed for DFT/AMBER calculations.

### Results

A rationally engineered TEVp variant with improved activity towards unfavored amino acids at P1′ using SenseNet and a random library strategy, has been developed.

SenseNet (Schneider, M. & Antes, I. SenseNet, a tool for analysis of protein structure networks obtained from molecular dynamics simulations. PLoS One 17, e0265194 (2022)) is an in-silico graph network tool developed in our group which enables analysis of the evolution of interaction networks between protein residues over time, as sampled with classical molecular dynamics (MD). Briefly, residues and their interactions are incorporated into nodes and edges, enabling the calculation of node and edge correlation factors (NCF/ECF) to find residues with a strong effect on the conformation of their surroundings. While the main application of SenseNet so far has been uncovering allosteric residues, we envisioned that its ability to find key influencers of local conformational dynamics could highlight mutation candidates to alter TEVp P1′ tolerance. To broaden the property space covered in our engineering efforts, the in silico suggestions were used as input for a random library to create a smart library. Random and smart libraries are commonly used in directed evolution experiments and involve the introduction of random mutations at random or selected positions of the target sequence. To progress on the state-of-the-art, the initial TEVp variant, termed “SM variant”, included all aforementioned engineering efforts. That is, the mutations T17S, N68D, I77V, R203G, and S219N, and deletion of the last five C-terminal amino acid residues (Δ238-242) (SEQ ID NO:31).

### Selecting Positions for Mutation

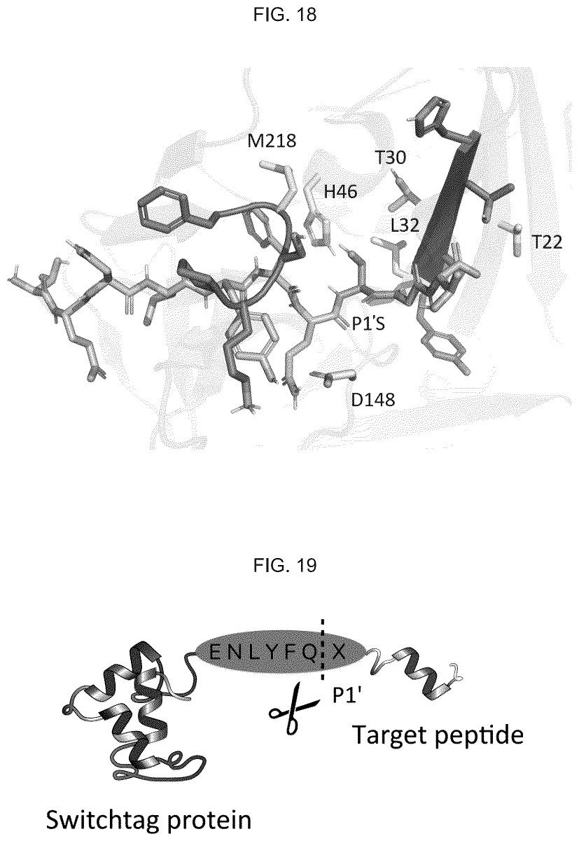

In the design of a smart library the challenge is first to identify key residue positions with high potential and second to mutate these in a limited way to keep the number of variants low. To identify positions of high potential to improve substrate specificity of the TEVp towards a mutated P1′ residue, the structural environment of this position was first carefully examined (FIG. 18). Manually mutating P1'S of the canonical recognition sequence in a crystal structure model of a substrate peptide bound to the TEVp (PDB 1LVB) showed that most mutations leading to enzymatic inactivity in experiment show steric clashes. In particular, most clashes occur with sidechains of T30, L32, and H46 (FIG. 19). This suggests these residues as clear mutation candidates to improve tolerance at the P1′ position, except for H46 as it is part of the catalytic triad and should thus be left unmodified. The initial analysis furthermore showed that bigger amino acids like W and Y clash with the enzyme backbone at the ²⁸HTTSLYGIG³⁶ beta sheet (SEQ ID NO:43) and ²¹⁷FMSKP²²¹ loop elements (SEQ ID NO:44) (FIG. 19). As these backbone clashes cannot be resolved by mutating the amino acids in these elements, more cryptic mutation candidates were explored.

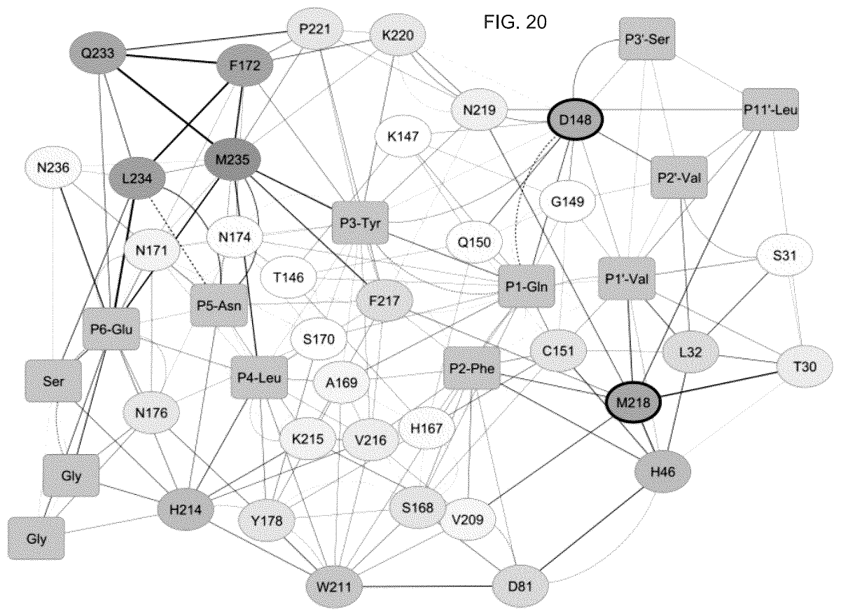

To this end the TEV protease was subjected to a graph network analysis with SenseNet. Herein, the SM variant TEVp was first simulated using MD with a Teriparatide-NUMA switch fusion construct substrate (see methods) bearing a P1′V instead of the canonical P1'S. This mutation was chosen as initial study case, since V is relatively small and should thus not require big conformational changes to be tolerated, yet it was previously identified as one of the disfavored residues at the P1′ position. All residues were then represented as nodes and the interactions between them as edges in CytoScape (v3.8.2). SenseNet was then used to calculate the edge and node correlation factors (ECF/NCF), which enabled visualization of residues that strongly affect the conformation of their surroundings (FIG. 20). Focusing on the peptide sequence and its nearest neighbors, D148 and M218 were identified as high-potential positions to enhance P1′V tolerance. These residues have a relatively strong conformational impact on their surroundings (darker grey nodes, FIG. 20) and make several interactions with a strong conformational effect (thick lines, FIG. 20). Moreover, they are in direct contact with the P1′V (P1′-Val, FIG. 20). M218 furthermore moderately affects T30 and H46's conformations, which were identified to cause putative clashes for some P1′ mutations in the initial mutation analysis.

To further assess the likelihood of these positions impacting the tolerance of P1′V, the structural relevance of D148 and M218 in the TEV protease were next assessed visually (FIG. 18). This showed that D148 and M218 are indeed located close to the P1′ position, being roughly below and above it, respectively. Moreover, M218 is part of the ²¹⁷FMSKP²²¹ loop that was identified before as an important element in P1′ tolerance. Finally, in conjunction with the network information, T22 was identified as an interesting mutation candidate to affect P1′ acceptance. This residue is located directly behind T30, which was already selected as a mutation candidate, and may therefore have synergistic effects if mutated along with it.

### Mutagenesis Strategy and Smart Library Design

A smart library comprising twenty TEVp variants was next generated containing random mutations at the positions identified by the afore-mentioned analyses: position 22, 30, 148, and 218.

### Screening Performance on the Smart Library

To test the effect of the selected mutations on TEVp cleavage efficiency, all TEVp-SM clones from the smart library were subjected to a target screening using two fluorogenic peptides. Namely, ENLYFQ-G peptide (SEQ ID NO:45), which served as a control screening, and ENLYFQ-V peptide (SEQ ID NO:46), which was the target screening. The parental type, TEVp-SM without mutations, showed no activity against the ENLYFQ-V peptide (SEQ ID NO:46) in microplate format, so the ENLYFQ-G peptide (SEQ ID NO:45) was used for initial screening optimization. 58 Mutation clones of TEVp-SM were assayed and an acceptable coefficient of variation (CV) of 15% was achieved (data not shown).

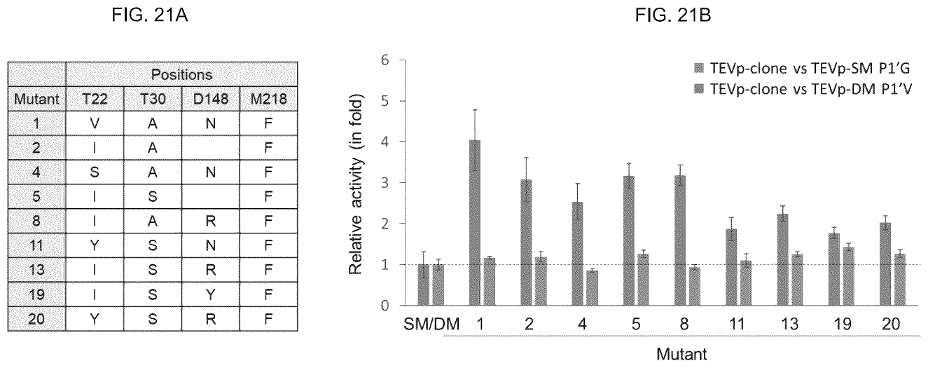

To estimate final screening expectations, a double mutant (DM) TEVp variant was tested with ENLYFQ-V peptide in microplate format. Unlike TEVp-SM, TEVp-DM displayed consistent activity, albeit low (data not shown), against the ENLYFQ-V peptide (SEQ ID NO:46). Since the aim was to optimize cleavage efficiency for P1′V, it was decided to incorporate TEVp-DM in the screening process to allow better comparisons. 896 individual clones, inoculated in 11 microplates, were screened with both fluorogenic peptides. In each plate column 6 was inoculated with TEVp-SM, while column 7 was inoculated with TEVp-DM. Considering parental type did not show activity against ENLYFQ-V peptide (SEQ ID NO:46), clones in the library were compared to TEVp-DM. 119 clones showed higher activity than the CV threshold (21%), while 78 clones displayed more than two-fold improvement over TEVp-DM (data not shown). The twenty best performing clones for the ENLYFQ-V peptide (SEQ ID NO:46) were then selected for a rescreening process. Of those twenty clones, nine unique mutants that showed significantly higher activity with the ENLYFQ-V peptide (SEQ ID NO:46) than TEVp-DM were identified, confirming the screening results (FIGS. 21A-21B). Interestingly, activity for the ENLYFQ-G peptide (SEQ ID NO:45) was preserved in all nine clones. The other eleven clones were repetitions, which was not surprising considering the nature of the library. Among winner mutants, especially clone 1 (TEVp-C1) stood out, with four-fold improvement for the ENLYFQ-V peptide (SEQ ID NO:46) compared to TEVp-DM (FIG. 21B).

### Validation of the Screening Hits

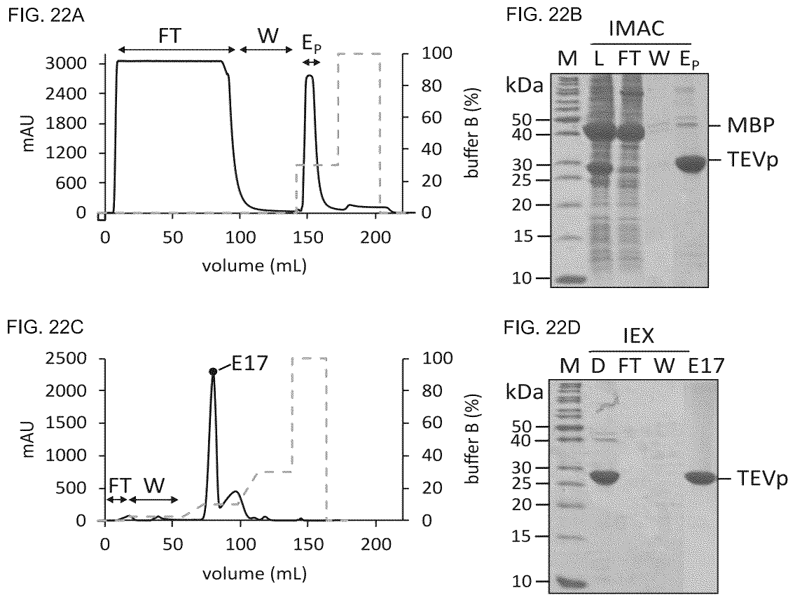

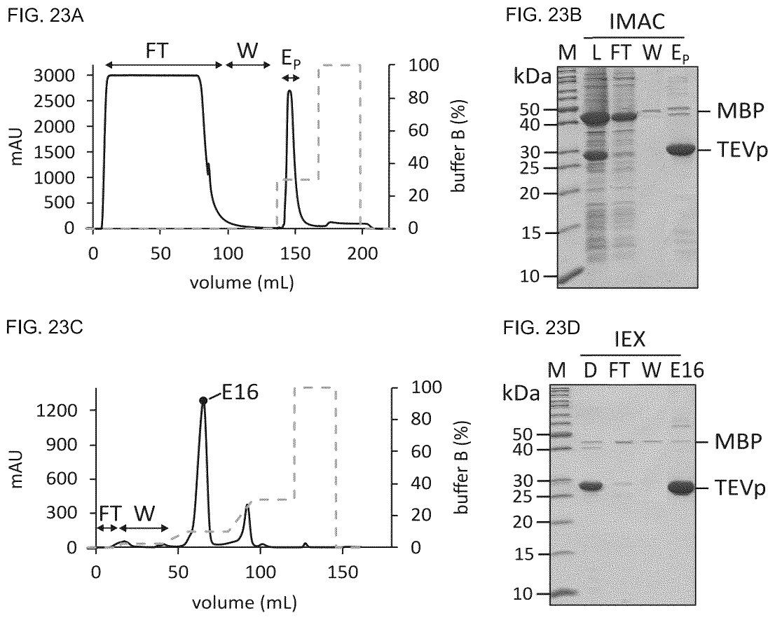

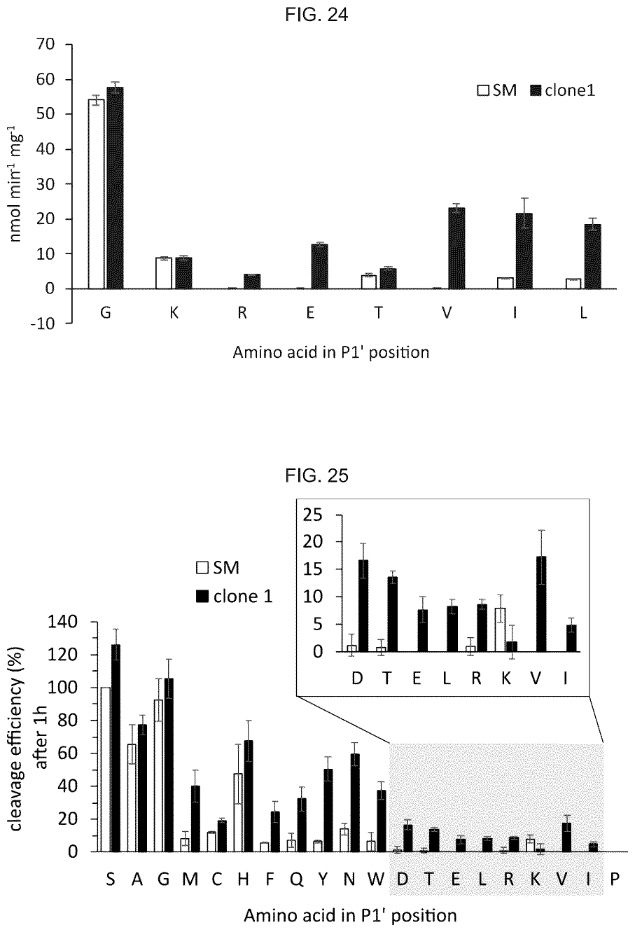

To verify the observed enhanced cleavage efficiency of TEVp-C1 for P1′V in the library mutant screenings and study the tolerance of this variant for other amino acids at the P1′ position, screening was reperformed with purified TEV proteases (>95% purity, FIGS. 22A-22D and 23A-23D). TEVp-SM and TEVp-C1 variants were tested against control substrate ENLYFQ-G (SEQ ID NO:45) and seven ENLYFQ peptides (SEQ ID NO:34) harboring the disfavored K, R, E, T, V, I and L at P1′. The specific activities of TEVp-C1 towards 25 μM peptide substrates were compared to TEVp-SM (FIG. 24). While TEVp-SM showed little to no activity against P1′ R, E, V, I and L, TEVp-C1 displayed markedly higher activities against these substrates implying improved specificities for charged and hydrophobic amino acids. Comparable specific activities for P1′G, K, and T substrates were achieved for both variants.

Inspired by the enhanced cleavage activities for these disfavored P1′ residues, a follow-up screen was performed to investigate P1′ specificity of TEVp-C1 against all twenty proteinogenic substrates. For this screen, an N-terminal Switchtag protein (HlyA fragment) containing the P1′ position was connected to a Teriparatide peptide through a TEVp recognition site. Cleavage efficiency was then measured by analyzing the amounts of Teriparatide peptide released from the P1′-containing Switchtag protein by TEVp-SM or TEVp-C1 using RP-HPLC (FIG. 25).

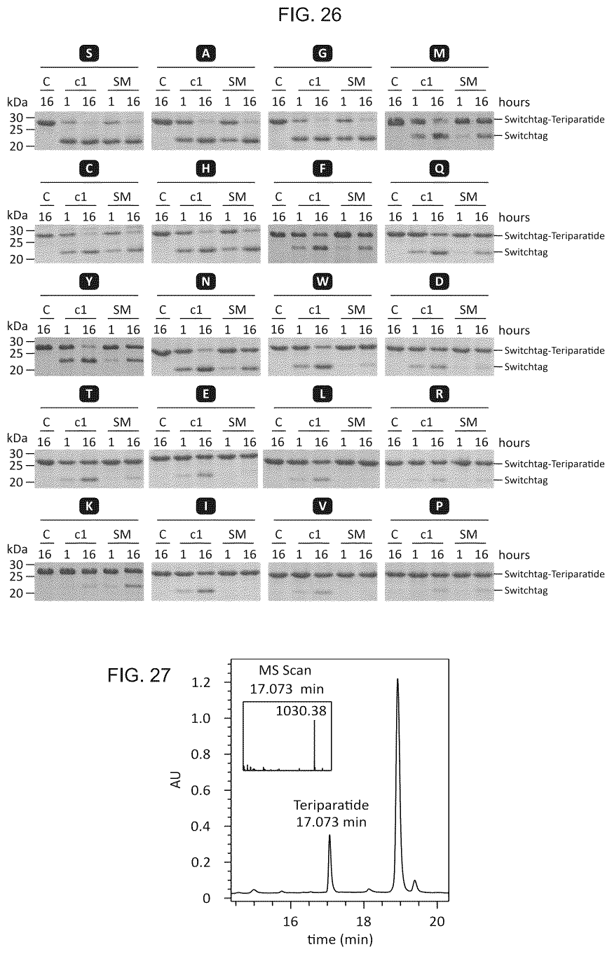

The identities of released Teriparatide targets were confirmed by mass spectrometry analysis (data not shown). Considering the size of these structures, the recognition site is assumed to be less accessible compared to the short fluorogenic peptides used in the previous screening, rendering them more suitable substrates to assess cleavage efficiencies under real conditions. For 18 out of 20 P1′ residues, all except K and P, the cleavage efficiency of TEVp-C1 after one hour was higher compared to TEVp-SM (FIG. 25). The highest improvements were observed for P1′ M, Y, N and W with cleavage efficiency increases ranging from 31-46%. These data align well with the results from the microtiter plate assay where superior performance of TEVp-C1 against fluorogenic peptides P1′ E, V, I and L was observed (FIG. 24). To study the long-term cleavage efficiencies, RP-HPLC analysis was also performed after 16 hours of incubation (FIG. 26). This highlighted even further the superior cleavage activity of TEVp-C1 against the majority of disfavored P1′ amino acids (e.g. F, Q, Y, N, D, T, V, 1). In particular, for P1′ W the cleavage efficiency was drastically improved for TEVp-C1 (90% compared to 9% for TEVp-SM).

In a separate approach, cleavage efficiency was studied by SDS-PAGE after 1 and 16 hours of incubation (FIG. 27) and densiometric quantification of fusion protein and Switchtag protein bands with the ImageJ software. This confirmed the RP-HPLC results as similar cleavage efficiencies were observed. Only for P1′ C contradictory results were observed. While densiometric measurements revealed that the fusion proteins were nearly completely cleaved after 16 hours of incubation (FIG. 26) product recoveries after RP-HPLC analysis of cleavage reactions were relatively low (12-23%) (FIG. 25). Indeed, further studies showed that proper product recovery was hampered by formation of cysteine dimerizations during cleavage reaction. Addition of the reducing agent DTT in the cleavage reactions inhibited disulfide bridge formations and considerably increased the product recovery in RP-HPLC analysis (data not shown).

### Understanding the Improved Tolerance of TEVp-C1

To uncover why TEVp-C1 exhibits such generally increased tolerance at the P1′ position compared to TEVp-SM, long MD simulations were performed under the experimental conditions of this study (see methods). The TEVp-SM and TEVp-C1 variants were simulated in the presence of the fluorogenic peptides used in experiment, containing P1′G or P1′V, resulting in four simulations of 6 μs each. To account for the possible bias of the starting positions of the introduced mutations (T22V, T30A, D148N, and M218F), only the last 4 μs of each trajectory was considered for final analysis.

Finally, the effect of the proposed mutations on the cleavage was studied by simulating the reaction mechanism in TEVp-SM with a P1′G or P1′V substrate and in TEVp-C1 with a P1′V substrate using a hybrid quantum mechanics/molecular mechanics (QM/MM) approach. Starting from the final structures generated by the long MD simulations, free energy surfaces were generated along distinguished reaction coordinates for each chemical step of the cleavage mechanism. This showed that it is the second step of the chemical transformation of the V-containing substrate that is compromised in TEVp-SM and is improved in the TEVp-C1. That is, the proton transfer from the catalytic H56 to the nitrogen of the P1′-X peptide bond.

All documents cited herein, are hereby incorporated by reference in their entirety. The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention. The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group. Further embodiments of the invention will become apparent from the following claims.

1. Polypeptide comprising an amino acid sequence that has at least 80%, at least 85%, at least 90%, at least 95%, at least 97.5%, at least 99% or at least 99.5% sequence identity over its entire length with the amino acid sequence set forth in SEQ ID NO:1 and comprises the amino acid substitution Q74L/V/I/F/W/Y/C and optionally any one or more of S135G/F, I138T, S153N/C/I/V, R203G/Q and M218F/I/W/L; wherein the positions 68, 77, 46, 81 and 151 are invariable, and the position 17 is S or A and the position 219 is N/V/D/E/P or K, wherein the positional numbering is according to SEQ ID NO:1, or a functional fragment thereof, wherein the polypeptide or the functional fragment has protease activity.

2. The polypeptide of claim 1, wherein the amino acid sequence that has at least 80%, at least 85%, at least 90%, at least 95%, at least 97.5%, at least 99% or at least 99.5% sequence identity over its entire length with the amino acid sequence set forth in SEQ ID NO:1 at its C-Terminus retains the deletion of amino acids 237-242 relative to the TEV protease wildtype sequence set forth in SEQ ID NO:2.

3. The polypeptide of claim 1, wherein the functional fragment is at least 202 amino acids in length and comprises amino acid residues corresponding to those at positions 17 to 218 of SEQ ID NO:1.

4. The polypeptide of claim 1, wherein the polypeptide comprises;
(1) the amino acid substitutions Q74L/V/I/F/W/Y/C and S135G/F, and optionally any one or more of I138T, S153N/C/I/V, R203G/Q and M218F/I/W/L;
(2) the amino acid substitutions Q74L, and S135G, and optionally any one or more of I138T, S153N, R203G and M218F;
(3) the amino acid substitutions Q74L/V/I/F/W/Y/C and I138T, and optionally any one or more of S135G/F, S153N/C/I/V, R203G/Q and M218F/I/W/L;
(4) the amino acid substitutions Q74L and I138T, and optionally any one or more of S135G, S153N, R203G and M218F;
(5) the amino acid substitutions Q74L/V/I/F/W/Y/C and S153N/C/I/V, and optionally any one or more of S135G/F, I138T, R203G/Q and M218F/I/W/L;
(6) the amino acid substitutions Q74L, and S153N, and optionally any one or more of S135G, I138T, R203G and M218F;
(7) the amino acid substitutions Q74L/V/I/F/W/Y/C and R203G/Q, and optionally any one or more of S135G/F, I138T, S153N/C/I/V and M218F/I/W/L;
(8) the amino acid substitutions Q74L, and R203G, and optionally any one or more of S135G, I138T, S153N and M218F;
(9) the amino acid substitutions Q74L/V/I/F/W/Y/C and M218F/I/W/L, and optionally any one or more of S135G/F, I138T, S153N/C/I/V and R203G/Q;
(10) the amino acid substitutions Q74L, and M218F, and optionally any one or more of S135G, I138T, S153N and R203G;
(11) the amino acid substitutions Q74L/V/I/F/W/Y/C, S135G/F and I138T, and optionally any one, any two or three of S153N/C/I/V, R203G/Q and M218F/I/W/L;
(12) the amino acid substitutions Q74L, S135G and I138T, and optionally any one, any two or three of S153N, R203G and M218F;
(13) the amino acid substitutions Q74L/V/I/F/W/Y/C, S135G/F and S153N/C/I/V, and optionally any one, any two or three of I138T, R203G/Q and M218F/I/W/L;
(14) the amino acid substitutions Q74L, S135G, and S153N, and optionally any one, any two or three of I138T, R203G and M218F;
(15) the amino acid substitutions Q74L/V/I/F/W/Y/C, S135G/F and R203G/Q, and optionally any one, any two or three of I138T, S153N/C/I/V and M218F/I/W/L;
(16) the amino acid substitutions Q74L, S135G, and R203G, and optionally any one, any two or three of I138T, S153N and M218F;
(17) the amino acid substitutions Q74L/V/I/F/W/Y/C, S135G/F and M218F/I/W/L, and optionally any one, any two or three of I138T, S153N/C/I/V and R203G/Q;
(18) the amino acid substitutions Q74L, S135G, and M218F, and optionally any one, any two or three of I138T, S153N and R203G;
(19) the amino acid substitutions Q74L/V/I/F/W/Y/C, I138T and S153N/C/I/V, and optionally any one, any two or three of S135G/F, R203G/Q and M218F/I/W/L;
(20) the amino acid substitutions Q74L, I138T and S153N, and optionally any one, any two or three of S135G, R203G and M218F;
(21) the amino acid substitutions Q74L/V/I/F/W/Y/C, I138T and R203G/Q, and optionally any one, any two or three of S135G/F, S153N/C/I/V and M218F/I/W/L;
(22) the amino acid substitutions Q74L, I138T and R203G, and optionally any one, any two or three of S135G, S153N and M218F;
(23) the amino acid substitutions Q74L/V/I/F/W/Y/C, I138T and M218F/I/W/L, and optionally any one, any two or three of S135G/F, S153N/C/I/V and R203G/Q;
(24) the amino acid substitutions Q74L, I138T and M218F, and optionally any one, any two or three of S135G, S153N and R203G;
(25) the amino acid substitutions Q74L/V/I/F/W/Y/C, S153N/C/I/V and R203G/Q, and optionally any one, any two or three of S135G/F, I138T, M218F/I/W/L;
(26) the amino acid substitutions Q74L, S153N, and R203G, and optionally any one, any two or three of S135G, I138T and M218F;
(27) the amino acid substitutions Q74L/V/I/F/W/Y/C, S153N/C/I/V and M218F/I/W/L, optionally any one, any two or three of S135G/F, I138T, R203G/Q;
(28) the amino acid substitutions Q74L, S153N, and M218F, and optionally any one, any two or three of S135G, I138T and R203G;
(29) the amino acid substitutions Q74L/V/I/F/W/Y/C, R203G/Q and M218F/I/W/L, and optionally any one, any two or three of S135G/F, I138T, S153N/C/I/V;
(30) the amino acid substitutions Q74L, R203G, and M218F, and optionally any one, any two or three of S135G, I138T and S153N;
(31) the amino acid substitutions Q74L/V/I/F/W/Y/C, S135G/F, I138T and S153N/C/I/V, and optionally any one or two of R203G/Q and M218F/I/W/L;
(32) the amino acid substitutions Q74L, S135G, I138T and S153N, and optionally any one or two of R203G and M218F;
(33) the amino acid substitutions Q74L/V/I/F/W/Y/C, S135G/F, I138T and R203G/Q, and optionally any one or two of S153N/C and M218F/I/W/L;
(34) the amino acid substitutions Q74L, S135G, I138T and R203G, and optionally any one or two of S153N and M218F;
(35) the amino acid substitutions Q74L/V/I/F/W/Y/C, S135G/F, I138T and M218F/I/W/L, and optionally any one or two of S153N/C/I/V and R203G/Q;
(36) the amino acid substitutions Q74L, S135G, I138T and M218F, and optionally any one or two of S153N and R203G;
(37) the amino acid substitutions Q74L/V/I/F/W/Y/C, S135G/F, S153N/C/I/V, and R203G/Q, and optionally any one or two of I138T and M218F/I/W/L;
(38) the amino acid substitutions Q74L, S135G, S153N and R203G, and optionally any one or two of I138T and M218F;
(39) the amino acid substitutions Q74L/V/I/F/W/Y/C, S135G/F, S153N/C/I/V and M218F/I/W/L, and optionally any one or two of I138T and R203G/Q;
(40) the amino acid substitutions Q74L, S135G, S153N and M218F, and optionally any one or two of I138T and R203G;
(41) the amino acid substitutions Q74L/V/I/F/W/Y/C, I138T, S153N/C/I/V and R203G/Q, and optionally any one or two of S135G/F and M218F/I/W/L;
(42) the amino acid substitutions Q74L, I138T, S153N, and R203G, and optionally any one or two of S135G and M218F;
(43) the amino acid substitutions Q74L/V/I/F/W/Y/C, I138T, S153N/C/I/V and M218F/I/W/L, and optionally any one or two of S135G/F and R203G/Q;
(44) the amino acid substitutions Q74L, I138T, S153N, and M218F, and optionally any one or two of S135G and R203G;
(45) the amino acid substitutions Q74L/V/I/F/W/Y/C, S153N/C, R203G/Q and M218F/I/W/L, and optionally any one or two of S135G/F and I138T;
(46) the amino acid substitutions Q74L, S153N, R203G, and M218F, and optionally any one or two of S135G and I138T;
(47) the amino acid substitutions Q74L/V/I/F/W/Y/C, S135G/F, R203G/Q, and M218F/I/W/L, and optionally any one or two of I138T and S153N/C/I/V;
(48) the amino acid substitutions Q74L, S135G, R203G, and M218F, and optionally any one or two of I138T and S153N;
(49) the amino acid substitutions Q74L/V/I/F/W/Y/C, S135G/F, I138T, S153N/C/I/V and R203G/Q, and optionally M218F/I/W/L;
(50) the amino acid substitutions Q74L, S135G, I138T, S153N, and R203G, and optionally M218F;
(51) the amino acid substitutions Q74L/V/I/F/W/Y/C, S135G/F, I138T, S153N/C/I/V and M218F/I/W/L, and optionally R203G/Q;
(52) the amino acid substitutions Q74L, S135G, I138T, S153N, and M218F, and optionally R203G;
(53) the amino acid substitutions Q74L/V/I/F/W/Y/C, S135G/F, I138T, R203G/Q and M218F/I/W/L, and optionally S153N/C/I/V;
(54) the amino acid substitutions Q74L, S135G, I138T, R203G, and M218F, and optionally S153N;
(55) the amino acid substitutions Q74L/V/I/F/W/Y/C, S135G/F, S153N/C/I/V, R203G/Q and M218F/I/W/L, and optionally I138T;
(56) the amino acid substitutions Q74L, S135G, S153N, R203G, and M218F, and optionally I138T;
(57) the amino acid substitutions Q74L/V/I/F/W/Y/C, I138T, S153N/C/I/V, R203G/Q and M218F/I/W/L, and optionally S135G/F;
(58) the amino acid substitutions Q74L, I138T, S153N, R203G and M218F, and optionally S135G;
(59) the amino acid substitutions Q74L/V/I/F/W/Y/C, S135G/F, I138T, S153N/C/I/V, R203G/Q and M218F/I/W/L; or
(60) the amino acid substitutions Q74L, S135G, I138T, S153N, R203G and M218F.

5. The polypeptide of claim 1, wherein the polypeptide comprises
(1) the amino acid substitutions Q74L/V/I/F/W/Y/C, S153N/C/I/V and R203G/Q;
(2) the amino acid substitutions Q74L, S153N and R203G;
(3) the amino acid substitutions Q74L/V/I/F/W/Y/C, S135G/F, S153N/C/I/V and R203G/Q;
(4) the amino acid substitutions Q74L, S135G, S153N and R203G;
(5) the amino acid substitutions Q74L/V/I/F/W/Y/C, I138T, S153N/C/I/V and R203G/Q;
(6) the amino acid substitutions Q74L, I138T, S153N and R203G;
(7) the amino acid substitutions Q74L/V/I/F/W/Y/C, S135G/F, I138T, S153N/C/I/V and R203G/Q;
(8) the amino acid substitutions Q74L, S135G, I138T, S153N and R203G;
(9) the amino acid substitutions Q74L/V/I/F/W/Y/C, S135G/F, I138T and R203G/Q;
(10) the amino acid substitutions Q74L, S135G, I138T and R203G;
(11) the amino acid substitutions Q74L/V/I/F/W/Y/C, S135G/F, I138T, S153N/C/I/V, R203G/Q and M218F/I/W/L; or
(12) the amino acid substitutions Q74L, S135G, I138T, S153N, R203G and M218F.

6. The polypeptide of claim 1, wherein the polypeptide further comprises any one or more of the amino acid substitution(s) L60W, F5C and L210C, wherein the positional numbering is according to SEQ ID NO:1.

7. The polypeptide of claim 1, wherein the polypeptide is an isolated polypeptide.

8. The polypeptide of claim 1, wherein
(a) the polypeptide is up to 236 amino acids in length; and/or
(b) the polypeptide comprises or consists of the amino acid sequence set forth in any one of SEQ ID Nos. 13 to 17, 24, 27, 28 or 30.

9. A composition comprising the polypeptide of claim 1.

10. A nucleic acid molecule encoding the polypeptide of claim 1.

11. A vector comprising the nucleic acid molecule according to claim 10.

12. A host cell comprising the nucleic acid molecule according to claim 10.

13. A method for the cleavage of a substrate polypeptide comprising the amino acid sequence motif set forth in SEQ ID NO:18 (ENLYFQX), comprising contacting the substrate polypeptide with the polypeptide of claim 1, under conditions that allow the cleavage of the polypeptide to generate a cleaved polypeptide.

14. (canceled)

15. The method of claim 13, wherein the substrate polypeptide is a fusion protein.

16. The method of claim 15, wherein the substrate polypeptide is a non-natural fusion protein.

17. The method of claim 14, further comprising purifying the cleaved polypeptide.

18. The host cell of claim 12, wherein the host cell is a prokaryotic host cell.

19. The polypeptide of claim 1, wherein the polypeptide comprises the amino acid substitutions Q74L, S135G, I138T, S153N, R203G and M218F.

20. The polypeptide of claim 19, wherein the amino acid sequence has at least 85% sequence identity over its entire length with the amino acid sequence set forth in SEQ ID NO:1.

21. The polypeptide of claim 19, wherein the amino acid sequence has at least 90% sequence identity over its entire length with the amino acid sequence set forth in SEQ ID NO:1.
